(19) United States
(12) Patent Application Publication
(10) Pub. No.: US 2020/0111552 A1
(43) Pub. Date: Apr. 9, 2020

(54) PATIENT DATABASE ANALYTICS

(71) Applicant: Masimo Corporation, Irvine, CA (US)

(72) Inventor: Omar Ahmed, Lake Forest, CA (US)

(21) Appl. No.: 16/594,803

(22) Filed: Oct. 7, 2019

Related U.S. Application Data
(60) Provisional application No. 62/742,781, filed on Oct. 8, 2018.

(51) Int. Cl. G16H 15/00; G06F 16/2458; G16H 10/60; G06F 16/248; G16H 40/67; G06F 9/451

(52) U.S. Cl. CPC G16H 15/00; G06F 16/2477; G06F 9/451; G06F 16/248; G16H 40/67; G16H 10/60

(57) ABSTRACT

Systems and methods are provided for improved analytics user interfaces. A clinician can use user interfaces and analytics to review different aspects of patient care in a clinical setting, such as information about one or more patients in the clinical setting. The improved user interfaces identify patients with physiological profiles as specified by a clinician. The improved user interfaces present physiological events for a patient with the particular physiological profiles. The improved user interfaces present trends and analytics of particular physiological parameters for a particular patient in a specific time range.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

This application claims benefit of U.S. Provisional Patent Application Ser. No. 62/742,781 entitled “Patient Database Analytics” filed Oct. 8, 2018, which is hereby incorporated by reference in its entirety.

### BACKGROUND

Hospitals, nursing homes, and other patient care settings typically include patient monitoring devices at one or more bedsides. Patient monitoring devices generally include sensors, processing equipment, and displays for presenting a medical patient's current physiological parameters. Physiological parameters include, for example, oxygen saturation (SpO2) level, respiratory rate, pulse, and blood pressure, among others.

The functionality of some existing patient monitoring devices typically is centered around a patient's current physiological parameters. This allows clinicians to have an understanding of a patient's current clinical status. Further, such patient monitoring devices typically have small form factors and limited interface options with respect to conducting patient analyses retrospectively or generating reports. Moreover, such patient monitoring devices typically do not include analytics functionality, such as the ability to query historical physiological parameters or other historical events captured by patient devices.

### SUMMARY

For purposes of summarizing the disclosure, certain aspects, advantages and novel features are discussed herein. It is to be understood that not necessarily all such aspects, advantages or features will be embodied in any particular embodiment of the invention and an artisan would recognize from the disclosure herein a myriad of combinations of such aspects, advantages or features.

One embodiment includes a system for investigating patient desaturations, the system comprising: data storage that stores a plurality of historical events, wherein each historical event from the plurality of historical events comprises an oxygen saturation parameter value for a patient at a timestamp; and a hardware processor configured to: receive, from a user interface, first user input comprising a first domain, the first domain indicating a group of patient devices; receive, from the user interface, second user input comprising a time range, the time range comprising a start time and an end time; receive, from the user interface, third user input comprising (i) an oxygen drop percentage, (ii) a time window, and (iii) a duration; execute a query comprising input parameters, the input parameters comprising the first domain, the start time, and the end time, wherein executing the query further comprises: identifying, from the plurality of historical events, a first subset of the historical events from the first domain and within the start time and the end time; identify, from the first subset of the historical events, a second subset of the historical events, wherein identifying the second subset of the historical events further comprises: identifying, for a particular patient, respective oxygen saturation physiological parameter values and timestamps within the oxygen drop percentage, the time window, and the duration; generate desaturation summary data comprising a quantity of desaturation events for each patient from the second subset of the historical events; and output the desaturation summary data for presentation in a desaturation summary user interface.

In some embodiments, the system of the preceding paragraph can include a combination or sub-combination of features. The desaturation summary user interface can include: for each patient from the second subset of the historical events, a patient indicator, a domain indicator, and a count indicator for the quantity of desaturation events; and wherein the hardware processor can be further configured to: receive, from the desaturation summary user interface, fourth user input comprising a selection for a first patient; and cause presentation of some data from the second subset of the historical events for the first patient in a desaturation detail user interface. The desaturation detail user interface can further include: a visual entry for a first desaturation event for the first patient, the visual entry comprising: (i) a first indicator representing a first duration of the first desaturation event, and (ii) a second indicator representing a first oxygen saturation value for the first desaturation event. The desaturation detail user interface can further include: a visual representation of a distribution for a plurality of desaturation events for the first patient grouped by a time unit.

One embodiment includes a method for generating patient analytics, the method comprising: under control of a hardware processor, receiving, from a user interface, first user input comprising a first domain, the first domain indicating a group of patient devices; receiving, from the user interface, second user input comprising a time range, the time range comprising a start time and an end time; executing (i) a first query comprising input parameters and (ii) and a second query comprising the input parameters, the input parameters comprising the first domain, the start time, and the end time, wherein executing the first query further comprises: identifying, from a plurality of historical events, a subset of the historical events from the first domain and within the start time and the end time, wherein executing the second query further comprises: identifying, from a plurality of notifications, a subset of the notifications from the first domain and within the start time and the end time, wherein each historical event from the plurality of historical events comprises a physiological parameter value for a patient at a timestamp; generating event summary data comprising a first quantity of historical events from the subset of the historical events; generating notification summary data comprising a second quantity of notifications from the subset of the notifications; and outputting the event summary data and the notification summary data for presentation in a dashboard user interface.

In some embodiments, the method of the preceding paragraph can include a combination or sub-combination of features. The dashboard user interface can further include: a first count indicator for the first quantity of historical events, a second count indicator for the second quantity of notifications, at least some historical events from the subset of the historical events, and at least some notifications from the subset of notifications. The method can further include: receiving, from the user interface, third user input comprising an updated time range; determining updated event summary data and updated notification summary data based at least in part on the updated time range; and outputting the updated event summary data and the updated notification summary data for presentation in an updated dashboard user interface. Generating the event summary data can further include: calculating, from the subset of the historical events, a third quantity of historical events corresponding to a first event category; and calculating, from the subset of the historical events, a fourth quantity of historical events corresponding to a second event category, wherein the event summary data further comprises the third quantity and the fourth quantity, and wherein the dashboard user interface further comprises: a third count indicator for the third quantity of historical events, and a fourth count indicator for the fourth quantity of historical events. Generating the notification summary data can further include: calculating, from the subset of the notifications, a third quantity of notifications corresponding to a first notification category; and calculating, from the subset of the historical events, a fourth quantity of notifications corresponding to a second notification category, wherein the notification summary data further comprises the third quantity and the fourth quantity, and wherein the dashboard user interface further comprises: a third count indicator for the third quantity of notifications, and a fourth count indicator for the fourth quantity of notifications. Generating the event summary data can further include: calculating, from the subset of the historical events, a third quantity of historical events corresponding to a first patient; calculating, from the subset of the historical events, a fourth quantity of historical events corresponding to a second patient; determining that the third quantity is greater than the fourth quantity; and selecting the third quantity for presentation instead of the fourth quantity, wherein the event summary data further comprises the third quantity, and wherein the dashboard user interface further comprises: a third count indicator for the third quantity of historical events. Generating the event summary data can further include: calculating, from the subset of the historical events, a third quantity of historical events corresponding to a first patient device for a first patient, wherein the event summary data further comprises the third quantity, and wherein the dashboard user interface further comprises: a third count indicator for the third quantity of historical events. The dashboard user interface can further include a patient dashboard user interface for a first patient.

One embodiment includes a system for generating patient analytics, the system comprising: data storage that stores a plurality of historical events and a plurality of notifications, wherein each historical event from the plurality of historical events comprises a physiological parameter value for a patient at a timestamp; and a hardware processor configured to: receive, from a user interface, first user input comprising a first domain, the first domain indicating a group of patient devices; receive, from the user interface, second user input comprising a time range, the time range comprising a start time and an end time; execute (i) a first query comprising input parameters and (ii) and a second query comprising the input parameters, the input parameters comprising the first domain, the start time, and the end time, wherein executing the first query further comprises: identifying, from the plurality of historical events, a subset of the historical events from the first domain and within the start time and the end time, wherein executing the second query further comprises: identifying, from the plurality of notifications, a subset of the notifications from the first domain and within the start time and the end time; generate event summary data comprising a first quantity of historical events from the subset of the historical events; generate notification summary data comprising a second quantity of notifications from the subset of the notifications; and output the event summary data and the notification summary data for presentation in a dashboard user interface.

In some embodiments, the system of the preceding paragraph can include a combination or sub-combination of features. The dashboard user interface can further include: a first count indicator for the first quantity of historical events, a second count indicator for the second quantity of notifications, at least some historical events from the subset of the historical events, and at least some notifications from the subset of notifications; and wherein the hardware processor can be further configured to: receive, from the user interface, third user input comprising an updated time range; determine updated event summary data and updated notification summary data based at least in part on the updated time range; and output the updated event summary data and the updated notification summary data for presentation in an updated dashboard user interface. A first notification from the plurality of notifications can include a message that is sent to a clinician device, wherein the message is indicative of a first physiological parameter exceeding an alarm threshold for a predetermined period of time. Generating the event summary data can further include: calculating, from the subset of the historical events, a third quantity of historical events corresponding to a first event category; and calculating, from the subset of the historical events, a fourth quantity of historical events corresponding to a second event category, wherein the event summary data further comprises the third quantity and the fourth quantity, and wherein the dashboard user interface further comprises: a third count indicator for the third quantity of historical events, and a fourth count indicator for the fourth quantity of historical events. The first event category can further include at least one of: a clinical event category, a non-clinical event category, or a modifier event category. The event summary data can further include: calculating, from the subset of the notifications, a third quantity of notifications corresponding to a first notification category; and calculating, from the subset of the historical events, a fourth quantity of notifications corresponding to a second notification category, wherein the notification summary data further comprises the third quantity and the fourth quantity, and wherein the dashboard user interface further comprises: a third count indicator for the third quantity of notifications, and a fourth count indicator for the fourth quantity of notifications. Generating the event summary data can further include: calculating, from the subset of the historical events, a third quantity of historical events corresponding to a first patient; calculating, from the subset of the historical events, a fourth quantity of historical events corresponding to a second patient; determining that the third quantity is greater than the fourth quantity; and selecting the third quantity for presentation instead of the fourth quantity, wherein the event summary data further comprises the third quantity, and wherein the dashboard user interface further comprises: a third count indicator for the third quantity of historical events. Generating the event summary data can further include: calculating, from the subset of the historical events, a third quantity of historical events corresponding to a first patient device for a first patient, wherein the event summary data further comprises the third quantity, and wherein the dashboard user interface further comprises: a third count indicator for the third quantity of historical events.

### BRIEF DESCRIPTION OF THE DRAWINGS

Throughout the drawings, reference numbers are re-used to indicate correspondence between referenced elements. The drawings are provided to illustrate embodiments of the inventions described herein and not to limit the scope thereof.

FIG. 1A depicts an example clinical computing environment that includes an analytics system.

FIG. 1B depicts example patient devices.

FIG. 1C depicts an example patient monitoring display.

FIG. 2 depicts an example group selector user interface for a clinician device.

FIG. 3 depicts an example time selector user interface for a clinician device.

FIG. 4 depicts another example time selector user interface for a clinician device.

FIG. 5 depicts an example desaturation parameter user interface for a clinician device.

FIG. 6 depicts an example desaturation user interface for a clinician device.

FIGS. 7A-7C depict additional example desaturation user interfaces for a clinician device.

FIG. 8 depicts an example desaturation analytics process.

FIG. 9A depicts an example analytics user interface for a clinician device.

FIG. 9B depicts an example trend analysis parameter selection user interface for a clinician device.

FIG. 10 depicts another example trend analysis parameter selection user interface for a clinician device.

FIG. 11A depicts another example analytics user interface for a clinician device.

FIGS. 11B-11C depict example trend analysis user interfaces for a clinician device.

FIG. 12 depicts an example trend analytics process.

FIGS. 13A-13B depict example dashboard analytics user interfaces for a clinician device.

FIGS. 14A-14D depict additional example analytics user interfaces for a clinician device.

FIG. 15 depicts another analytics user interface for a clinician device.

FIGS. 16A-16C depict example dashboard analytics user interfaces for a clinician device.

FIG. 17 depicts another example time selector user interface for a clinician device.

FIGS. 18A-18B, 19A-19B, 20A-20C, 21A-21D depict additional example analytics user interfaces for a clinician device.

FIG. 22 depicts an example export user interface for a clinician device.

FIGS. 23A-23C, 24A-24C, 25A-25H, 26A-26C, 27A-27B depict example analytics documents.

FIG. 28 depicts an example user interface analytics process.

FIG. 29 is a block diagram illustrating an operating environment for an example analytics system.

### DETAILED DESCRIPTION

### I. Introduction

Patient monitoring devices can be used to improve medical care for patients. The patient monitoring devices typically present a patient's current physiological parameters or other patient events. Clinicians, including doctors, nurses, physician's assistants, and other medical personnel can use the current physiological parameters obtained from the patient to diagnose illnesses and to prescribe treatments. Clinicians also can use the current physiological parameters to monitor a patient's status during various clinical situations to determine whether to increase the level of medical care given to the patient. However, as opposed to exclusively using current patient events, using historical physiological parameters or other historical events from patient devices can advantageously provide insights into improving patient care that may otherwise not be detected. Thus, analytic systems and methods can benefit medical care for patients by providing improved user interfaces that use historical events from patient devices.

The present disclosure relates to analytics systems and methods for user interfaces that can improve patient care. A clinician can use improved user interfaces to review different aspects of patient care in a clinical setting, including information about one or more patients in the clinical setting. A clinician may be interested in the following questions, for example: Within a particular area, who are the patients that fit low oxygen saturation profiles as specified by a clinician? What are the particular low oxygen or desaturation events for a patient with a low oxygen profile? What are the trends of particular physiological parameters for a particular patient in a specific time range? Additional examples of issues that can be addressed with the analytics systems and methods described herein are described below.

In particular, a clinician can use the improved user interfaces to identify a subset of patients or a particular patient and generate analytics data for the identified patient(s). One or more input parameters from a clinician can be used to generate the analytics user interfaces. Example input parameters include a time range, patient identifier, a domain, one or more physiologically related parameters, or some combination thereof. The analytics system or method can identify historical events based on the input parameters. The analytics system or method can further generate and present summary data in one or more user interfaces based on the identified historical events. A clinician can view the results and obtain answers for their clinically related questions. The clinician or the analytics system can then take corrective measures based on the identified or generated data, such as increasing or decreasing patient alarms or reallocating resources or personnel to improve patient care.

As mentioned above, the functionality of some existing patient monitoring devices typically is centered around a patient's current physiological parameters. Also as mentioned above, such patient monitoring devices typically have small form factors and limited interface options with respect to conducting patient analyses retrospectively or generating reports. Moreover, such patient monitoring devices typically do not include analytics functionality, such as the ability to query historical physiological parameters or other historical events captured by patient devices.

The present disclosure describes analytics systems and methods that can result in improved display user interfaces. The improved user interfaces can allow a clinician to more quickly access desired data. The improved user interfaces may enable clinicians to access data more quickly especially in the case where the user interfaces are implemented on devices with small form factors and/or small display areas. The data that can be accessed quickly can include, but is not limited to, analytics related to patients with low oxygen profiles, a subset of low oxygen or desaturation events for patient, or trends in particular physiological parameters for a patient, to name a few. The present disclosure describes a particular manner for identifying display information. The present disclosure also describes a particular manner of summarizing and presenting information in electronic device. A clinician may be unable to access desired data with some existing patient monitoring devices. Further, without the analytics systems and methods described herein, it may be possible to cobble together the desired data in an ad-hoc manner; however, such solutions may require manual steps and may not include the specific data or functionality of the analytics systems and methods described herein. Such ad-hoc solutions may be slow, complex, and difficult to use for clinicians.

### II. Example Clinical Computing Environment

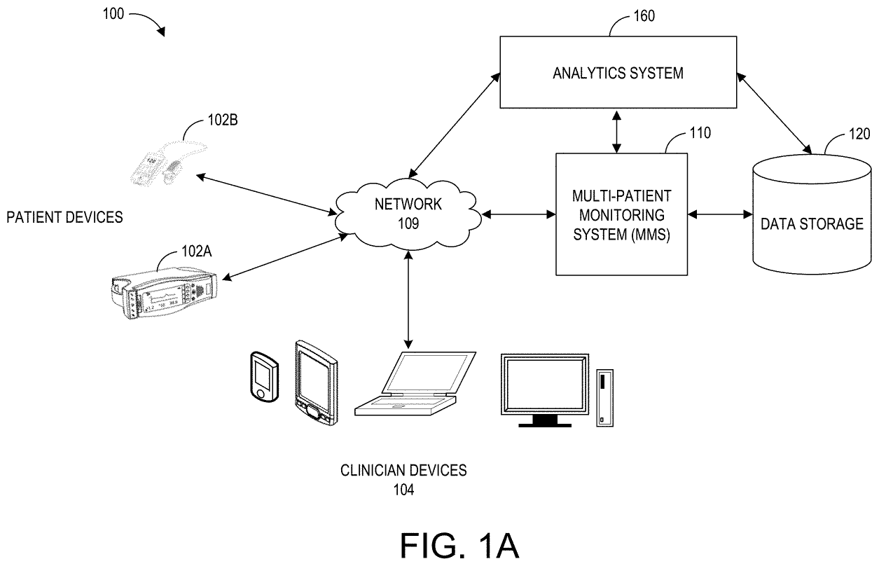

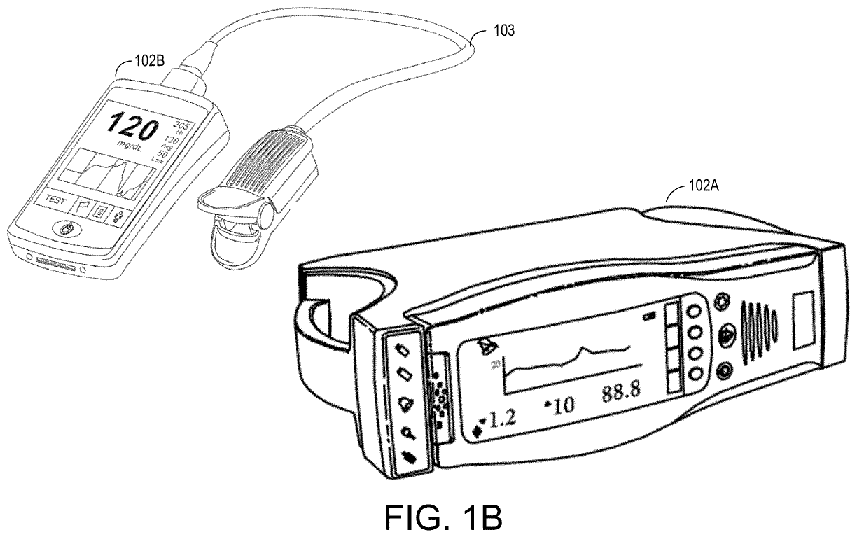

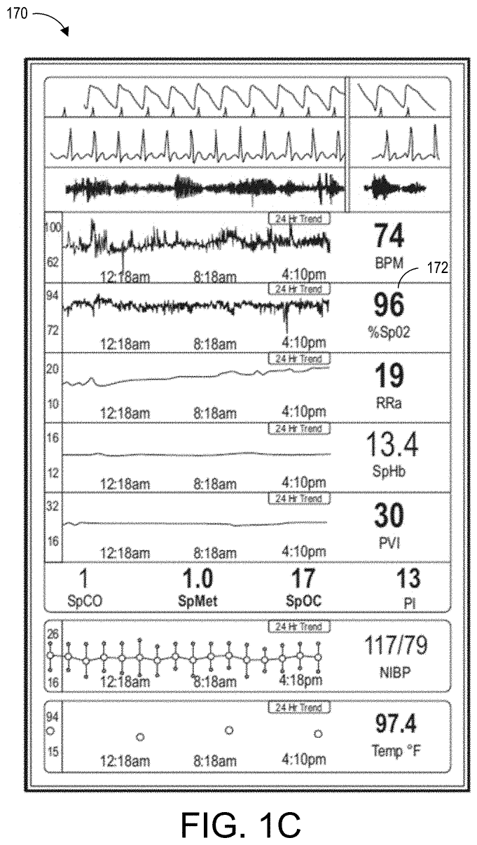

Turning to FIG. 1A, an example clinical computing environment 100 is shown. The clinical computing environment 100 may be implemented in one or more hospitals or other clinical settings. Further, the clinical computing environment 100 can facilitate remotely analyzing patients, such as by using network-enabled monitoring equipment.

In the clinical computing environment 100, various patient devices 102A, 102B and clinician devices 104 communicate over a network 109 with a multi-patient monitoring system (MMS) 110. The MMS 110 can include a remote server that can communicate with patient devices 102A, 102B and clinician devices 104. The network 109 may include a local area network (LAN), a wide area network (WAN), a public network (such as the Internet), a private network, or any combination of the same. For instance, the network 109 can include a wireless and/or wired hospital network or a network that connects multiple clinical facilities. As another example, a patient device 102A, 102B can connect with the MMS 110 from a patient's home, over the network 109. In that situation, the network 109 may be a hospital network that exposes a virtual public network (VPN) connection to the patient devices 102A, 102B. Further, the MMS 110 may be implemented in a cloud infrastructure that permits remote connection from patient devices 102A, 102B at home or in any other location. For convenience, this specification primarily describes patient data or patient device data as being routed through the MMS 110 to the data storage 120. However, in other examples, the patient devices 102A, 102B can communicate directly with the data storage 120.

The patient devices 102A, 102B may be any of the patient monitors or monitoring devices described herein and may include bedside monitors, ambulatory or mobile monitors, in-home monitors, and the like. The patient devices 102A, 102B can be point-of-care devices, such as bedside devices or patient-worn devices. The patient devices 102A, 102B can receive input from physiological sensors or the patient devices 102A, 102B can include physiological sensors. The physiological sensors can be coupled with a patient and may measure parameters such as oxygen saturation or SpO2, respiratory rate, blood pressure, heart rate or pulse rate perfusion, other blood gas parameters, brain activity, brain oxygen saturation, any of the other parameters described herein, and the like. The patient devices 102A, 102B can provide information about a patient's status, including current values of physiological parameters, waveforms, trend values, and historical values of physiological parameters over the network 109 to the MMS 110. The MMS 110 can in turn store this data in a data storage system 120. An analytics system 160 can retrieve historical data, such as historical values of physiological parameters or other historical events, from the data storage 120. The analytics system 160 can generate user interfaces that provide insight into patients, patient devices, or clinical settings based on the historical data. In contrast to real-time or near-time monitoring, the user interfaces generated by the analytics system 160 can provide retrospective views of patient or patient device data, such as by using historical data. The analytics system 160 can include one or more servers to retrieve data, perform analyses, or generate user interfaces.

The analytics system 160 can provide historical data, such as via one or more user interfaces, to the clinician devices 104. The clinician devices 104 can include any mobile device, such as a laptop, tablet, cell phone, smartphone, personal digital assistant (PDA), or any other device. In some cases, the clinician devices can include desktop systems. In addition to receiving historical data from the analytics system 160, the clinician devices 104 can receive notifications from the patient devices 102A, 102B through the MMS 110. When a patient device 102A, 102B detects that a parameter of a patient has exceeded a threshold set in the patient device 102A, 102B (or otherwise triggered an alarm condition), the patient device 102A, 102B can send an alarm over the network 109 to the MMS 110. In turn, the MMS 110 can send the alarm or a message representing the alarm to the clinician devices 104.

### III. Example Patient Monitoring of Current Physiological Parameters and Historical Data

FIG. 1B depicts, in an enlarged view, the example patient devices 102A, 102B of FIG. 1A. As described above with respect to FIG. 1A, the patient device 102A of FIG. 1B can be a patient monitor or a monitoring device, such as, but not limited to, a bedside monitor, ambulatory or mobile monitor, or an in-home monitor. Also as described above with respect to FIG. 1A, the patient device 102A or 102B of FIG. 1B can include or be connected to a physiological sensor, such as the physiological sensor 103.

The physiological sensor 103 can include or be a pulse oximetry device or an acoustic respiration monitor. Pulse oximetry provides a noninvasive procedure for measuring the oxygen status of circulating blood and may be used in a wide variety of medical contexts, such as surgical wards, intensive care units, neonatal units, general wards, home care, physical training, clinics, and emergency medical situations. A pulse oximetry system generally includes a physiological sensor applied to a patient, a monitor, and a cable connecting the sensor and the monitor. The sensor has light emitters and a detector, which are attached to a tissue site, such as a finger. The cable can transmit emitter drive signals from the monitor to the sensor where the emitters respond to the drive signals to transmit light into the tissue site. The detector is responsive to the emitted light after attenuation by pulsatile blood flowing in the tissue site. The detector outputs a detector signal to the monitor. The monitor processes the detector signal to provide a numerical readout of physiological parameters such as oxygen saturation (SpO2) and pulse rate.

Enhanced oximetry systems can also include a multiple parameter monitor and a multiple wavelength sensor that can provide enhanced measurement capabilities, including the measurement of a multitude of blood constituents and related parameters in addition to oxygen saturation and pulse rate, such as, carboxyhemoglobin (HbCO), methemoglobin (HbMet), total Hematocrit (Hct), total hemoglobin (Hbt), oxygen concentrations, glucose concentrations, blood pressure, electrocardiogram data, temperature, respiratory rate, and/or acoustic respiration rate (RRa®), as a few examples. Advanced physiological monitors and multiple wavelength optical sensors capable of measuring parameters in addition to SpO2, such as HbCO, HbMet, Hct, and/or Hbt are described in at least U.S. patent application Ser. No. 11/367,013, filed Mar. 1, 2006, titled Multiple Wavelength Sensor Emitters, now issued as U.S. Pat. No. 7,764,982, and U.S. patent application Ser. No. 11/366,208, filed Mar. 1, 2006, titled Noninvasive Multi-Parameter Patient Monitor, now issued as U.S. Pat. No. 8,130,105, which are hereby incorporated by reference in their entireties. Further, noninvasive blood parameter monitors and optical sensors including Rainbow™ adhesive and reusable sensors and RAD57™, Radical-7™, Root™, and other monitors capable of measuring SpO2, pulse rate, perfusion index (PI), signal quality (SiQ), pulse variability index (PVI), HbCO and/or HbMet, among other parameters, are also commercially available from Masimo Corp. of Irvine, Calif.

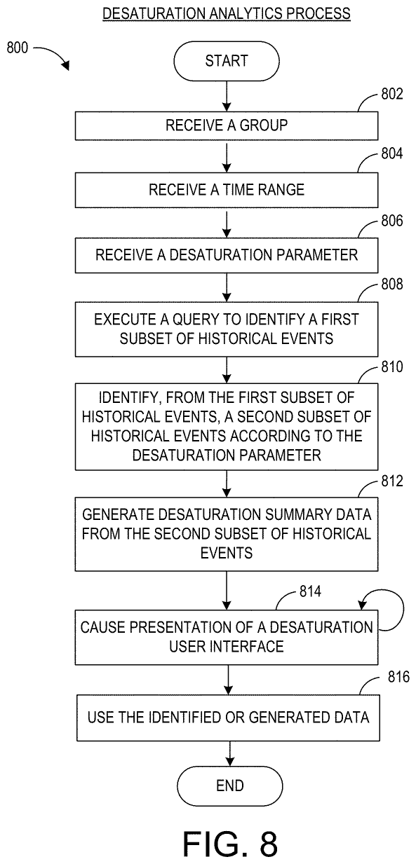

FIG. 1C depicts an example patient monitoring display 170 that may be presented by the patient devices 102A, 102B described above. The patient monitoring display 170 may present a wide variety of measurement data in numerical, graphical, waveform, or other display format. The patient monitoring display 170 can display current physiological parameters for a patient, such as the current oxygen saturation percentage 172 for the patient. As shown, the captured physiological parameters, such as an oxygen related physiological parameter value for a patient, can be recorded over time as historical events, such as by being recorded as a time series that includes data value and timestamp pairs. A historical event can include a parameter, a state, or some metadata regarding a patient device that is associated with a time, such as a timestamp. The historical events can be stored in the data storage 120. Moreover, the historical events can be stored with metadata, such as identifiers for: the device that captured the physiological parameter or other data, where the device was located, the patient, or other information about the patient or the device. The analytics system 160 can use the historical events for analytics purposes, examples of which are described in further detail below with respect FIGS. 8, 12, and 28.

Historical events can include clinical events and notifications. A clinical event can be recorded when a physiological parameter exceeds an alarm threshold. A notification can be recorded when the clinical event (or a non-clinical or modifier event) occurs for a time longer than an alarm delay. The notification, such as a message, can be sent from a first device (such as the patient device 102A, 102B or the MMS 110) to a second device (such as the clinician device 104). The message can be indicative of a physiological parameter exceeding an alarm threshold for a predetermined period of time. For example, a notification can occur when a patient's physiological parameter goes above or below a threshold. The patient device can accordingly emit a bedside alarm, and if the alarm is not cleared for a predetermined period of time, then a notification can be issued to alert one or more clinician devices in the clinical setting. Example notifications can include initial notifications, escalation notifications, and re-escalation notifications. Additional details regarding notifications are described in at least U.S. Provisional Patent Application No. 62/712,154, filed Jul. 30, 2018, titled Mobile Patient Alarm Display, which is hereby incorporated by reference in its entirety.

Some historical data, such as historical events, can include non-clinical data. Example non-clinical data may not be related to a physiological parameter, but instead may include information about the state of a patient device 102A, 102B. Example information about the state of a patient device can indicate that a sensor that has been disconnected or otherwise has fallen off (often referred to as a probe-off condition). Likewise, a brief power outage or surge can cause the patient device 102A, 102B to transmit an indication of the event, an indication that the patient device 102A, 102B has reset, or a non-clinical alarm indication. Additional example non-clinical events are described below in

TABLE 1

| Non-Clinical Event | Event Description |
|---|---|
| All Mute | The patient device is in an “All Mute” state. |
| No Sensor | A sensor is not detected as being attached to the patient cable. |
| Defective Sensor | Sensor appears to be defective. |
| Interference | Ambient light interference is detected by sensor. |
| Sensor Off | The sensor is off the patient. |
| Unrecognized Sensor | The patient device does not recognize sensor. |
| Check Sensor | Check the sensor placement to ensure that emitter and detector |
| | are in alignment. |
| No Cable Connected | Cable is disconnected from the patient device. |
| Incompatible Cable | The patient device is incompatible with cable. |
| Unrecognized Cable | The patient device does not recognize cable. |
| Defective Cable | Cable is defective. |
| Emitter Temp Out of | The temperature of the sensor emitter is out of range. |
| Range | |
| Sensor Current Limit | The sensor has exceeded the current limit. |
| Exceeded | |
| No Adhesive | The patient device does not detect adhesive. |
| Invalid Adhesive | The patient device detects invalid adhesive. |
| Defective Adhesive | The adhesive is defective. |
| No Acoustic Sensor | The patient device does not detect that an acoustic sensor is |
| Connected | connected. |
| Defective Acoustic Sensor | The acoustic sensor is defective. |
| Respiratory Pause | The sensor detects a respiratory pause longer than the user |
| | defined time period. |
| Acoustic Sensor Off | The acoustic sensor is disconnected from the patient. |
| Patient | |
| Bad Acoustic Sensor | The acoustic sensor is incorrectly placed. |
| Placement | |
| Unrecognized Acoustic | The patient device does not recognize the acoustic sensor. |
| Sensor | |
| Incompatible Acoustic | The acoustic sensor is incompatible with the patient device. |
| Sensor | |
| No Acoustic Cable | There is not acoustic cable connected. |
| Connected | |
| Incompatible Acoustic | The acoustic cable is incompatible with the patient device. |
| Cable | |
| Unrecognized Acoustic | The patient device does not recognize acoustic cable. |
| Cable | |
| Defective Acoustic Cable | The acoustic cable is defective. |
| Low Battery | The patient device battery is low. |

Historical events can include modifier events. A modifier event can indicate an environmental state, such as the environmental state of a patient device that can affect monitored parameters, but may not generate alarms by themselves. Example modifier events are described below in Table 2.

TABLE 2

| Modifier | |
|---|---|
| Event | Description |
| Patient | Acoustic sensor detects interference from the patient. |
| Interference | |
| Background | Acoustic sensor detects interference from the room. |
| Interference | |
| Low SIQ | The patient device detects a low signal quality indicator (SIQ) at sensor |
| | site. |
| Low | The patient device detects low perfusion at sensor site. |
| Low PR SIQ | The patient device detects low pulse rate SIQ at sensor site. |
| Low EtCO2 | The patient device detects a low capnography (EtCO2) SIQ at the sensor |
| SIQ | site. |
| Low RR | The patient device detects low confidence of respiration rate at sensor site. |
| Confidence | |
| Low RR | The patient device detects weak signal of respiration rate (RR) at sensor |
| Signal | site. |
| SpHb Low | The patient device detects low confidence of total hemoglobin (SpHb ®) at |
| Confidence | sensor site. |
| SpCO Low | The patient device detects low confidence of carboxyhemoglobin (SpCO ®) |
| Confidence | at sensor site. |
| SpMet Low | The patient device detects low confidence of methoglobin (SpMet ®) at |
| Confidence | sensor site. |
| Low FiCO2 | The patient device detects low fractional CO2 (FiCO2) SIQ at sensor site. |
| SIQ | |
| Low PVI SIQ | The patient device detects low Pleth Variability Index SIQ at sensor site. |
| Low PI SIQ | The patient device detects low Perfusion Index SIQ at sensor site. |
| Low SpOC | The patient device detects low oxygen content (SpOC ™) SIQ at sensor |
| SIQ | site. |
| Low PSI SIQ | The patient device detects low PSI SIQ at sensor site. |

### IV. Example Analytics User Interfaces

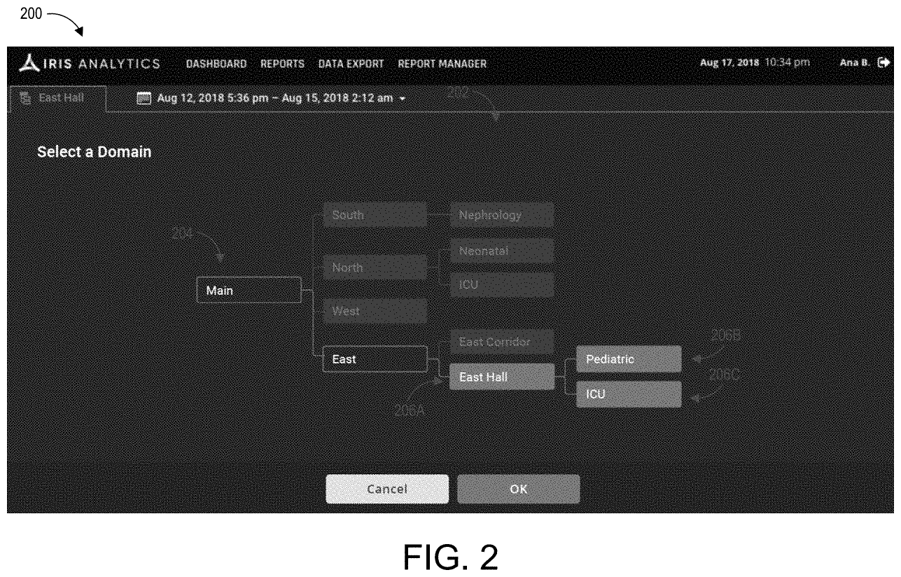

FIGS. 2 through 7A-7C, 9A-9B, 10, 11A-11C, and 13A-13B through 22 depict example clinician device user interfaces. These user interfaces can be presented by the clinician devices 104 described above using, for example, patient or patient device data received from the MMS 110 or the data storage 120. The MMS 110 can obtain the patient or patient device data from the patient devices 102A, 102B. The user interfaces shown may be generated by the analytics system 160 of FIG. 1A. Thus, each of the user interfaces shown may be output for presentation by electronic hardware as graphical user interfaces.

Each of the user interfaces shown includes one or more user interface elements or controls that can be selected by a user. The user interface elements shown are merely illustrative examples and can be varied in other embodiments. For instance, any of the user interface elements shown may be substituted with other types of user interface elements. Some examples of user interface elements that may be used include buttons, dropdown boxes, select boxes, text boxes or text fields, checkboxes, radio buttons, toggles, breadcrumbs (e.g., identifying a page or interface that is displayed), sliders, search fields, pagination elements, tags, icons, tooltips, progress bars, notifications, message boxes, image carousels, modal windows (such as pop-ups), date and/or time pickers, accordions (e.g., a vertically stacked list with show/hide functionality), and the like. Additional user interface elements not listed here may be used. Further, the user interfaces shown may be combined or divided into other user interfaces such that similar functionality or the same functionality may be provided. Moreover, each of the user interface elements may be selected by a user using one or more input options, such as a mouse, touch screen input (e.g., finger or pen), or keyboard input, among other user interface input options.

FIG. 2 depicts an example group selector user interface 200 that may be presented by the clinician devices 104 described above. The group selector user interface 200 can allow a clinician to configure subsequent user interfaces, such as by presenting data in subsequent user interfaces from patient devices from one or more groups. One or more patient devices can be assigned to a group. An example group can correspond to a particular geographical area of a clinical facility. Other criteria can be used to specify a group of patient devices. A group of patient devices can include one or more sub-groups. The one or more sub-groups can each further include one or more sub-groups, etc. A group of patient devices can include a hierarchy, such as a first group with children groups, grandchildren groups, and great-grandchildren groups. If a particular group or sub-group of patient devices is selected, then subsequent user interfaces may display data associated with the configured group while excluding other data associated with other groups. A clinical benefit of configuring a user interface for a particular group of patient devices can include limiting the quantity of data that can be displayed or accessed by the clinician, which can thereby improve the user interface by making data or information more easily accessible by the clinician and/or by making the user interface less cluttered. A “domain,” in addition to having its ordinary meaning, is used herein to refer to a type of grouping of patient devices or patients, such as by referring to a physical location associated with the group of patient devices or patients. The particular example domains shown in FIG. 2 include a “Main” domain that can refer to the patient devices or patients in the “South,” “North,” “West,” and “East” areas or domains in a clinical setting. The “South” domain can refer to the patient devices or patients in the “Nephrology” area or domain in the clinical setting. The “North” domain can refer to the patient devices or patients in the “Neonatal” and North “ICU” areas or domains in the clinical setting. The “East” domain can refer to the patient devices or patients in the “East Corridor” and “East Hall” areas or domains in the clinical setting and so forth.

The group selector user interface 200 can include a group selector 202. The group selector 202 can include a hierarchical group representation 204 that includes user-selectable group nodes. As shown in the group selector user interface 200, the “East Hall” group 206A is selected, which can cause subsequent user interfaces to include data associated with the selected group. Moreover, subsequent user interfaces may exclude data from non-selected groups. Also shown in the group selector user interface 200 in this example, the “East Hall” group 206A can include the “Pediatric” and “ICU” sub-groups 206B, 206C. Accordingly, since the parent “East Hall” group is selected in this example, subsequent user interfaces can include data associated with the “Pediatric” and “ICU” sub-groups 206B, 206C. The physical areas for the Pediatric and East Hall ICU areas in the clinical facility may be located within the East Hall of the facility. A clinician can select other group nodes, which may also be hierarchical, such as the Main, South, Nephrology, North, Neonatal, North ICU, West, East, and East Corridor nodes. The depicted nodes in FIG. 2 are examples and can vary according to a particular facility's layout.

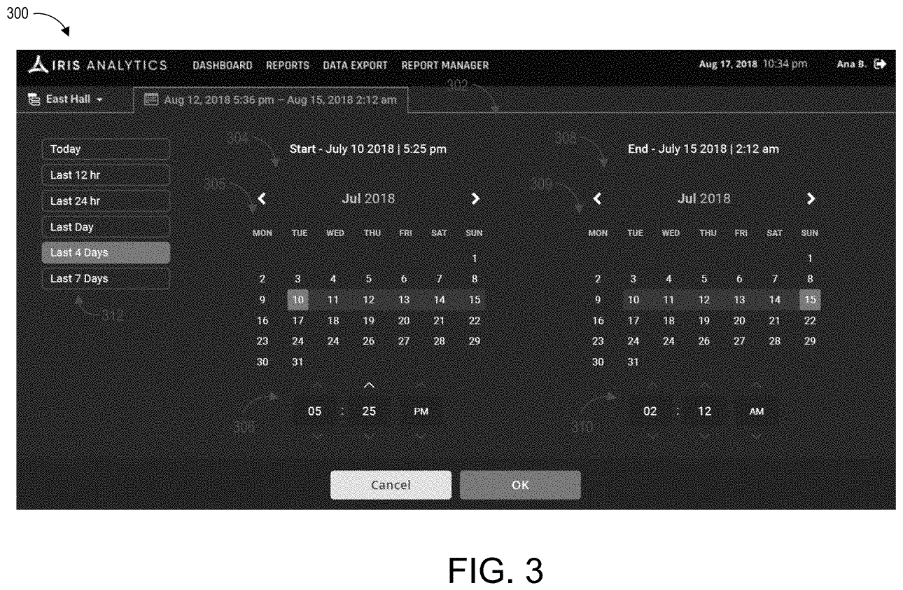

FIG. 3 depicts an example time selector user interface 300 that may be presented by the clinician devices 104 described above. The time selector user interface 300 can allow a clinician to configure subsequent user interfaces, such as by presenting data in subsequent user interfaces within a selected time range. In contrast to some existing patient monitors, the time selector user interface 300 can enable a historical view of patient data or patient device data as opposed to some existing patient monitors that strictly present current patient data or current patient device data.

The time selector user interface 300 can include a time selector 302. The time selector 302 can include a start time selector 304 and an end time selector 308. The start time selector 304 can include one or more of a start date component selector 305 and a start time component selector 306. The end time selector 308 can include one or more of an end date component selector 309 and a start time component selector 310. The time selector 302 can also include selectable options 312 for predetermined time ranges, such as, but not limited to, “Today,” “Last 12 hr,” “Last 24 hr,” “Last Day,” “Last 4 Days,” and “Last 7 days.” A selection of one of the selectable options 312 can update the time selector user interface 300 based on a current time. Subsequent to selection of a predetermined time range via the selectable options 312, a clinician can further adjust the time range with one of the start time selector 304 or the end time selector 308. As shown in the example time selector user interface 300, the selected time range can correspond to a start time of Jul. 10, 2018 at 5:25 PM and an end time of Jul. 15, 2018 at 2:12 AM.

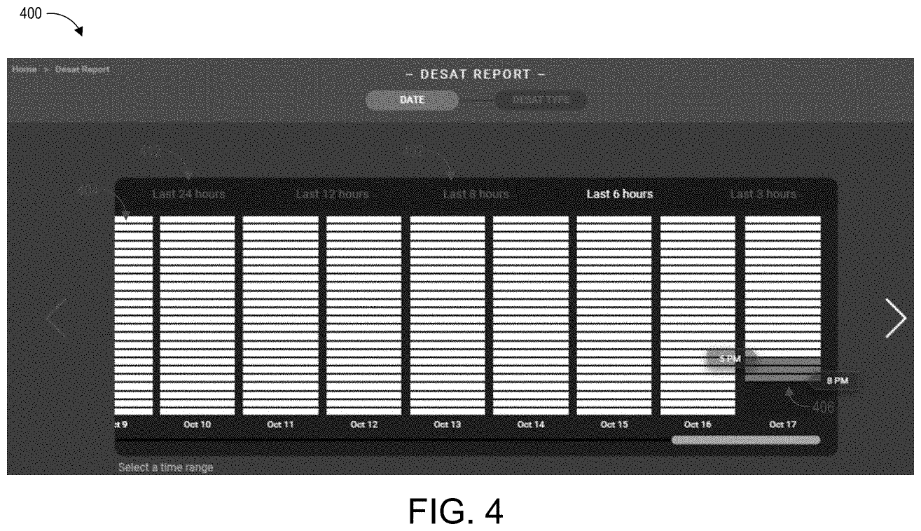

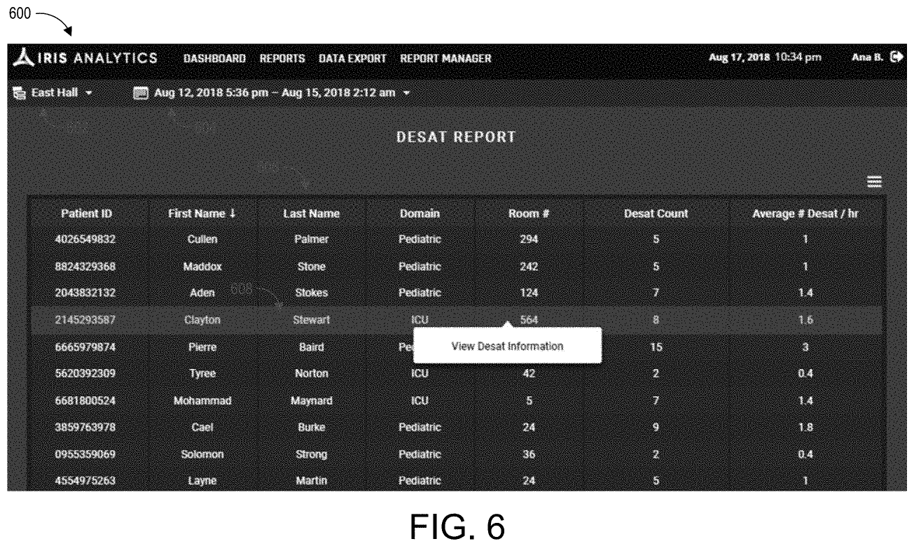

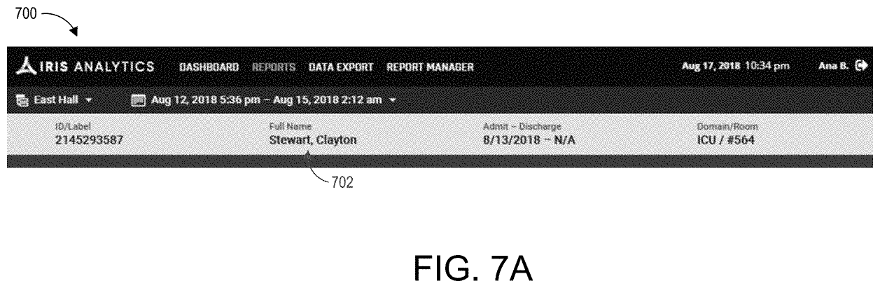

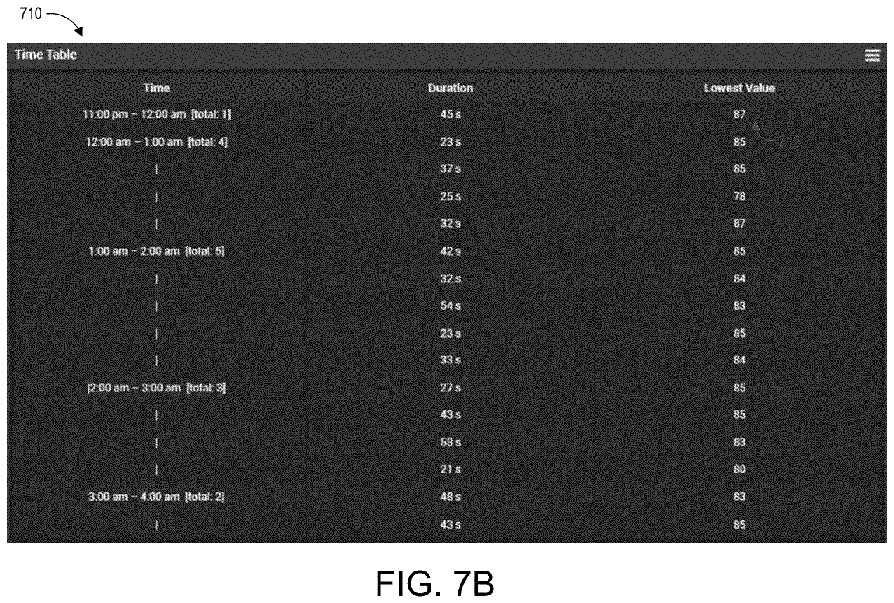

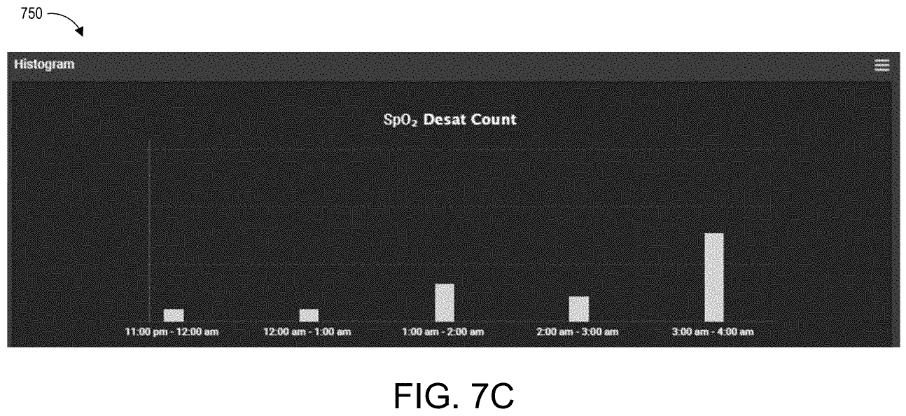

FIG. 4 depicts an example time selector user interface 400 that may be presented by the clinician devices 104 described above. The time selector user interface 400 of FIG. 4 can be similar to the time selector user interface 300 of FIG. 3. Accordingly, similar to the time selector user interface 300 of FIG. 3, the time selector user interface 400 of FIG. 4 can allow a clinician to configure subsequent user interfaces, such as by presenting data in subsequent user interfaces within a selected time range. However, the time selector user interface 400 of FIG. 4 can be used to select the time range for a desaturation user interface, such as a desaturation report, which is described in further detail below with respect to FIGS. 6 and 7A-7C.

A desaturation user interface can advantageously improve patient care. Desaturation can refer to situations where a patient has low oxygen saturation, which can be a serious clinical event. A desaturation user interface can enable a clinician to identify or review historical desaturation events or historical summary data associated with desaturation. Once historical events or historical summary data associated with desaturation have been identified or reviewed, prophylactic steps can be taken to improve patient care. In response to identification of one or more desaturation situations, a clinician or a healthcare system can proceed with one or more corrective measures, such as setting or updating an alarm for one or more patient devices to cause an alert upon detection of a desaturation situation. Further, in response to identification of one or more desaturation situations, any number of other corrective measures can be put into place in a clinical setting, such as adjusting or increasing the clinical staff or other resources in a physical location where the desaturation situations have been detected. Accordingly, a desaturation user interface can advantageously cause patient care to be improved.

The time selector user interface 400 can include a time selector 402. The time selector 402 can include a time segment selector 404. Each time segment, represented as a bar, can be for an hour. In particular, the selected time segments 406 can correspond to three hours, namely, 5 PM to 8 PM on October 17. The time selector 402 can also include selectable options 412 for predetermined time ranges, such as, but not limited to, “Last 24 hours,” “Last 12 hours,” “Last 8 hours,” “Last 6 hours,” and “Last 3 hours.” The selectable options 412 of FIG. 4 can be similar to the selectable options 312 of FIG. 3. Accordingly, selecting one of the selectable options 412 can update the time selector user interface 400 based on a current time. Subsequent to selection of a predetermined time range via the selectable options 412, a clinician can further adjust the time range with one of the time segment selector 404. As shown, the selected time range 406 can correspond to 5 PM to 8 PM on October 17, where the start time can be October 17 at 5 PM and the end time can be October 17 at 8 PM (in this example).

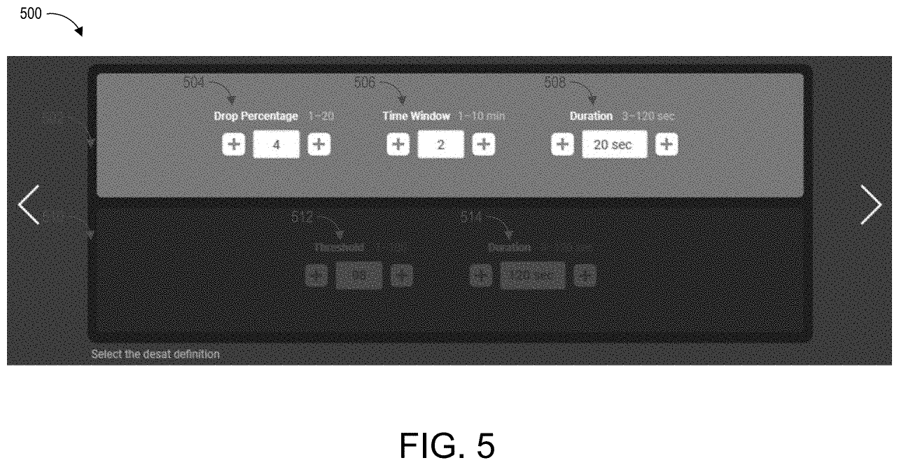

FIG. 5 depicts an example desaturation parameter user interface 500 that may be presented by the clinician devices 104 described above. The desaturation parameter user interface 500 can be used to receive input parameter(s) for a desaturation user interface, such as a desaturation report, which is described in further detail below with respect to FIGS. 6 and 7A-7C. The desaturation parameter user interface 500 can include a drop percentage input element 504, a time window input element 506, a first duration input element 508, a threshold input element 512, and a second duration input element 514. A clinician can adjust the input parameter(s) using one of the drop percentage input element 504, the time window input element 506, the first duration input element 508, the threshold input element 512, or the second duration input element 514. A clinician can define a low oxygen profile by selecting one or more of the desaturation parameters associated with the elements 504, 506, 508, 512, 514.

The desaturation parameter user interface 500 can receive user input for one or more of the drop percentage input element 504, the time window input element 506, the first duration input element 508, the threshold input element 512, and the second duration input element 514 that specify desaturation parameter(s) for a desaturation user interface, such as a desaturation report. The drop percentage input element 504 can receive an input value that indicates a drop in a particular unit of measurement, such as a percentage specifying a drop in oxygen saturation, which can have readings from 0 to 100%. The specified input value for the drop percentage input element 504, such as an oxygen drop percentage, can indicate a drop percentage within historical data that can be used to identify one or more desaturation events. The time window input element 506 can receive an input value that indicates a time window within historical data that can be used to identify one or more desaturation events. The time window input element 506 can receive an input value in a particular time unit (shown in the desaturation parameter user interface 500 in minutes), such as seconds, minutes, hours, or other time unit. The first duration input element 508 or the second duration input element 514 can receive an input value that indicates a time duration within historical data that can be used to identify one or more desaturation events. Similar to the time window input element 506, the duration input element 512, 514 can receive an input value in a particular time unit (shown in the desaturation parameter user interface 500 in seconds), such as seconds, minutes, hours, or other time unit. The threshold input element 512 can receive an input value that indicates a threshold in a particular unit of measurement, such as a percentage specifying a threshold for oxygen saturation, which can have readings from 0 to 100%. The specified input value for the threshold input element 512, such as an oxygen threshold percentage, can indicate a particular threshold within historical data that can be used to identify one or more desaturation events.

The desaturation parameter user interface 500 can include a predetermined grouping of the input parameters for a desaturation user interface. An example predetermined grouping of input parameters can include a first set of desaturation parameters 502 that includes parameters for the drop percentage input element 504, the time window input element 506, the first duration input element 508. The particular example input values shown for the drop percentage input element 504, the time window input element 506, and the first duration input element 508 (4%, 2 minutes, and 20 seconds, respectively) can indicate that desaturation events may be identified by the desaturation user interface where there is historical data that corresponds to at least the drop percentage in oxygen saturation (here 4%), within the time window (here 2 minutes), and that lasts for at least the duration (here 20 seconds). Another example predetermined grouping of input parameters can include a second set of desaturation parameters 510 that includes parameters for the threshold input element 512 and the second duration input element 514. As shown, if selected by a clinician, the particular input values for the threshold input element 512 and the second duration input element 514 (98% and 120 seconds, respectively) can indicate that desaturation events may be identified by the desaturation user interface where there is historical data that corresponds to at least an oxygen saturation below the threshold (here 98%) for at least the duration (here 120 seconds).

FIG. 6 depicts an example desaturation user interface 600 that may be presented by the clinician devices 104 described above. The desaturation user interface 600 can present desaturation summary data 606 that includes desaturation data for one or more patients. The desaturation user interface 600 can be a desaturation summary user interface. The desaturation user interface 600 can be generated based on input parameters received from other user interfaces, such as the user interfaces 200, 300, 400, 500 of FIGS. 2, 3, 4, and 5, respectively, to query historical events from one or more patients. A clinician can provide query parameters, such as, one or more group, one or more time ranges, or one or more desaturation parameters, that are used to derive the query results presented in the desaturation user interface 600. The desaturation user interface 600 can present the desaturation summary data 606 based on the selected group 602 (here “East Hall”) or a first selected time range 604 (here a time range with a start time of Aug. 12, 2018 at 5:36 PM and an end time of Aug. 15, 2018 at 2:12 AM). The desaturation user interface 600 can present the desaturation summary data 606 based on selected desaturation parameters, such as the parameters discussed above with respect to FIG. 5. Example query parameters associated with the desaturation summary data 606 can include at least a drop percentage in oxygen saturation (such as 4%), within a time window (such as 2 minutes), and that lasts for at least a duration (such as 20 seconds). Additional query parameters associated with the desaturation summary data 606 can include at least an oxygen saturation below a threshold (such as 98%) for at least a duration (such as 120 seconds). Thus, a clinician can improve patient care based on the desaturation summary data 606, such as by adjusting or increasing the clinical staff or other resources where desaturation situations have been detected.

As shown, the desaturation user interface 600 can present, for each patient, one or more patient identifiers, a group identifier (such as a domain identifier), and one or more indicators associated with desaturation events. The desaturation entry 608 can include one or more patient indicators, such as a patient number or string (here “2145293587”) or the patient's first or last name. The desaturation entry 608 can include a group indicator (here “ICU”) for the patient that can indicate a location of the patient. The desaturation entry 608 can include various desaturation summary indicators, such as a value for a “Desat Count” that can be a count indicator for the quantity of desaturation events for the patient (here the example desaturation count is eight desaturation events for the selected patient). Additional desaturation summary indicators include statistical measures for the desaturation events, such as an average, median, mode, weighted average, weighted median, weighted mode, or standard deviation. As shown, the desaturation entry 608 can include a statistical measure of desaturation events per time unit, such as an average number of desaturation events per hour (here 1.6).

FIGS. 7A-7C depict additional example desaturation user interfaces 700, 710, 750 that may be presented by the clinician devices 104 described above. The desaturation user interfaces 700, 710, 750 of FIGS. 7B and 7C can be presented in response to a user selection of the desaturation user interface 600 of FIG. 6. In FIG. 6, a clinician can select an entry, such as the entry 608 for the patient, Clayton Stewart, to cause presentation of desaturation data associated with the selected entry. One or more of the desaturation user interfaces 700, 710, 750 of FIGS. 7A, 7B, and 7C, respectively, can be presented by the clinician devices 104. The desaturation user interfaces 700, 710, 750 of FIGS. 7A, 7B, and 7C, respectively, can be presented together or separately. In some configurations, the desaturation user interface 700 of FIG. 7A can be shown above the desaturation user interface 710 of FIG. 7B, or the desaturation user interface 710 of FIG. 7B can be shown above the desaturation user interface 760 of FIG. 7C. The desaturation user interfaces 710, 750 of FIGS. 7B and 7C can be desaturation detail user interfaces by providing further details regarding desaturation events, such as further details regarding desaturation events for a particular patient.

Turning to FIG. 7A, the desaturation user interface 700 can include a patient identification section 702. The patient identification section 702 can identify the patient for the desaturation data described below with respect to FIGS. 7B and 7C. The patient identification section 702 can include a patient identifier, a name of the patient, a group of the patient, or a location of the patient. The identified patient can correspond to the selected entry 608 of the desaturation user interface 600 of FIG. 6.

Turning to FIG. 7B, the desaturation user interface 710 can include one or more visual entries for desaturation events. The depicted desaturation events can be for the selected patient or entry 608 from the desaturation user interface 600 of FIG. 6. A first visual entry 712 can be for a first desaturation event. The first visual entry 712 can include: (i) a first indicator representing a first duration of the first desaturation event (here 45 seconds), and (ii) a second indicator representing a first saturation value for the first desaturation event (here a lowest oxygen saturation value of 87% for the first desaturation event). The first visual entry 712 can correspond to the input parameters described above with respect to FIGS. 2-6, such that the first visual entry 712 or the other visual entries can fall within the previously specified input parameters. The visual entries may be grouped by a time unit (such as hourly shown here). As shown, four desaturation events can be grouped together within a first time period of 12:00 AM to 1:00 AM. The visual entries shown in the desaturation user interface 710 of FIG. 7B are illustrative and may not correspond exactly to the counts shown in the desaturation user interface 600 of FIG. 6.

Turning to FIG. 7C, the desaturation user interface 750 can include a visual representation of a distribution for desaturation events for a particular patient grouped by a time unit. Similar to the entries from the desaturation user interface 710 of FIG. 7B, the visual representation of desaturation events from FIG. 7C can be for the selected patient or entry 608 from the desaturation user interface 600 of FIG. 6. An example visual representation is a histogram of respective counts of desaturation events for a particular patient grouped by a time unit (here an hourly grouping). Similar to the entries from the desaturation user interface 710 of FIG. 7B, the visual representation of the counts shown in the desaturation user interface 750 of FIG. 7C are illustrative and may not correspond exactly to the counts shown in the desaturation user interface 600 of FIG. 6.

### V. Example Desaturation Analytics Process

Turning to FIG. 8, an example desaturation analytics process 800 is shown. The desaturation analytics process 800 may be implemented by the analytics system 160. For convenience, the desaturation analytics process 800 will be described in the context of the analytics system 160, although other computing systems not described herein may implement desaturation analytics process 800. In certain examples, the desaturation analytics process 800 can advantageously provide improved patient care by enabling identification or enabling more rapid identification of patient desaturation issues or enabling further actions, such as the implementation of corrective measures, as described herein.

At block 802, a group can be received. In particular, the analytics system 160 can receive a group, which can be a group identifier. As described above with respect to the user interface 200 of FIG. 2, a user interface can receive user input and the user input can include a group. Each patient device or patient may be associated with one or more groups and the association between groups and patient devices or patients can be stored in the data storage 120. An example group can include a domain that refers to a physical location associated with a group of patient devices or patients. A group identifying a subset of patient devices or patients can be used to identify a subset of historical events that can be used for desaturation analytics. Identifying a subset of desaturation analytics may be advantageous because determining or presenting desaturation analytics for a larger set of patient devices or patients may be computationally more expensive or impractical, may result in a more cluttered user interface, or may make it slower for a clinician to access desired data.

At block 804, a time range can be received. In particular, the analytics system 160 can receive a time range. A time range can include a start time and an end time. As described above with respect to the user interfaces 300, 400 of FIGS. 3 and 4, respectively, a user interface can receive user input, and the user input can include a time range. Similar to the use of a group at block 802, the time range can be used to identify a subset of historical events that can be used for desaturation analytics, such as the historical events that occurred or that includes a timestamp within the start time and end time of the time range. Again, similar to the advantages of using a group to identify a subset of patient devices or patients, there can be advantages to using a time range to identify a subset of historical events, such as increasing computational efficiency or even feasibility, making a user interface less cluttered, or making it faster for a clinician to access desired data.

At block 806, one or more desaturation parameters can be received. In particular, the analytics system 160 can receive a desaturation parameter. The desaturation parameters can be used to identify a subset of historical events, which can be used for analytics purposes. As described above with respect to FIG. 5, an example desaturation parameter can include a drop percentage, a time window, a duration, or a threshold. A clinician can define the desaturation parameters, such as a threshold and a time period. The analytics system 160 can identify a desaturation event if a patient's physiological parameters exceed the threshold for the time period.

Relative to a desaturation event being defined by desaturation parameters set by a clinician, a desaturation event can be defined in a more dynamic manner, such as by using adaptive threshold alarms. An adaptive alarm can be responsive physiological parameters, such as by adapting to baseline drift in the physiological parameter. A physiological parameter baseline can be generated from a physiological parameter trend. Physiological parameter limits can specify an allowable range of the physiological parameter. An adaptive threshold can be calculated from the physiological parameter baseline and the physiological parameter limits. A desaturation event can be generated based on the physiological parameter exceeding the adaptive threshold. The adaptive threshold can be responsive to the parameter baseline so as to increase in value as the physiological parameter baseline drifts to a higher physiological parameter value and to decrease in value as the physiological parameter baseline drifts to a lower parameter value. For example, the analytics system 160 can take a statistical measure of oxygen saturation for a patient for a time window, such as an average oxygen saturation for the patient for 120 seconds. If the oxygen saturation drops by a particular percentage value from the statistical measure of oxygen saturation, then the analytics system 160 can identify a desaturation event. Thus, the desaturation parameters can be calculated dynamically, such as by being based on a patient's baseline. Additional details regarding adaptive thresholds, which may be used with any of the systems described herein, are described in U.S. patent application Ser. No. 13/037,184, filed Feb. 28, 2011, titled Adaptive Alarm System, now issued as U.S. Pat. No. 9,724,024, which is hereby incorporated by reference in its entirety.

At block 808, a query can be executed to identify a subset of historical events. In particular, the analytics system 160 can execute a query to identify a first subset of historical events. As described above with respect to FIGS. 1A and 1C, the data storage 120 can be configured to store historical events. Each historical event in the data storage 120 can include an oxygen related physiological parameter value for a patient at a timestamp. The query can include input parameters, such as a group, a start time, or an end time. Executing the query can include identifying, from the historical events in the data storage 120, a first subset of historical events from the group and within the start time and the end time. The data storage 120 can include a database, such as a relational database or an object-oriented database, and the query can be in a format configured for the database, such as in a SQL format or an object-oriented database query format. For example, the query can include input parameters for a group, such as the domain “East Hall,” and a start time (such as Aug. 12, 2018 at 5:36 PM) and an end time (such as Aug. 15, 2018 at 2:12 AM). Executing the query can result in identifying historical events (such as oxygen related physiological parameter values for a patient at respective timestamps) that were captured by a device or for a patient associated with the group within the start time and the end time. This can enable the clinician to advantageously view historical events or summary data of those events to determine desaturation issues.

At block 810, a second subset of historical events can be identified from the first subset of historical events. In particular, the analytics system 160 can identify the second subset of historical events from the first subset of historical events. The analytics system 160 can identify the second subset of historical events using the one or more desaturation parameters. As described above with respect to block 806, a desaturation parameter can include a drop percentage, a time window, a duration, or a threshold. The analytics system 160 can identify, for a particular patient, respective oxygen-related physiological parameter values and timestamps within the desaturation parameter. Moreover, the analytics system 160 can identify, from the first subset of historical events, those historical events that fall within the desaturation parameter.

The analytics system 160 can identify, from the first subset of historical events, those historical events that fall within a particular set of desaturation parameters. A first set of desaturation parameters can include an oxygen drop percentage, a time window, and a first duration. Where the desaturation parameters include particular values for the oxygen drop percentage, the time window, and the first duration (such as 4%, 2 minutes, and 20 seconds, respectively), the analytics system 160 can identify, from the first subset of historical events (which can be for historical events from a particular group within a particular start time and end time), those oxygen related physiological parameter values that indicate a drop of at least the drop percentage (4%) and that occurred within the time window (2 minutes) for at least the first duration (20 seconds). A second set of desaturation parameters can include a threshold and a second duration. Where the desaturation parameters include particular values for the threshold and the second duration, such as 98%, 120 seconds, respectively, the analytics system 160 can identify, from the first subset of historical events, the oxygen related physiological parameter values that are below the threshold (98%) for at least the second duration (120 seconds).

The below table, Table 3, provides an example set of historical events, where each value can be an oxygen related physiological parameter value at a particular time.

TABLE 3

| Value | Time |
|---|---|
| 90 | 1 |
| 91 | 2 |
| 90 | 3 |
| . . . | . . . |
| 90 | 120 |
| 89 | 121 |
| 88 | 122 |
| 87 | 123 |
| 86 | 124 |
| 85 | 125 |
| 85 | 126 |
| . . . | . . . |
| 86 | 142 |
| . . . | . . . |
| 99 | 300 |
| 99 | 301 |
| . . . | . . . |

For the example with the first set of desaturation parameters, which can include the oxygen drop percentage, the time window, and the first duration, the analytics system 160 can process a subset of historical events within the time window to identify an oxygen drop percentage within the duration. The above table can represent the first subset of historical events and can be from a larger set of historical events, such as being the result of a query to select a subset of events within a time range or group. The above timestamps in the example table can correspond to seconds for illustrative purposes; however, the timestamps can also correspond to other time units, such as a finer or greater resolution of milliseconds, microseconds, minutes, etc. As shown in the above table, the historical values between timestamps 1 and 120 may stay consistent around 90% and may not have a drop in oxygen percentage. However, starting at the timestamp 121 until the timestamp 142, there can be a drop in oxygen percentage of at least 4%, which can correspond to specific desaturation parameters of the drop percentage (4%) that occurred within the time window (2 minutes) for at least the first duration (20 seconds). In some embodiments, the analytics system 160 can apply a sliding window to search for historical events that correspond to the first set of desaturation parameters.

For the example with the second set of desaturation parameters, which can include the threshold and the second duration, the analytics system 160 can process a subset of historical events to identify those events that are below the threshold for at least the second duration. As shown in the above table, the historical values between timestamps 1 and 120 may be below the threshold (98%) for at least the duration (120 seconds). Thus, the analytics system 160 can identify the historical events between timestamps 1 and 120 as corresponding to the second set of desaturation parameters. The analytics system 160 can continue processing historical events after the timestamp 120 to determine if there any additional historical events within the first subset that correspond to the second set of desaturation parameters.

In some embodiments, blocks 808 and 810 can be combined. In particular, instead of identifying the first subset of historical events at block 808 and the second subset of historical events at block 810, a single subset of historical events can be determined together. The analytics system 160 can execute a query to determine a single subset of historical events. The query can include any of the parameters, such as a group, a time range, or a desaturation parameter, from the previous blocks 802, 804, 806. The query can include one or more desaturation parameters to determine the subset of historical events, and the query can be in a format configured for a database, such as in a SQL format or an object-oriented database query format.

At block 812, desaturation summary data can be generated from the second subset of historical events. In particular, the analytics system 160 can generate desaturation summary data from the second subset of historical events. The analytics system 160 can calculate a quantity of desaturation events from the second subset of historical events. As described above with respect to block 810, the second subset of historical events can reflect the desaturation parameter(s), such as by indicating those historical event(s) that correspond to some combination of a drop percentage, a time window, a duration, or a threshold, thereby identifying a desaturation event. The analytics system 160 can quantify the identified desaturation events, such as by calculating statistical summary data that can include a total number of desaturation events or an average number of desaturation events per time unit. The analytics system 160 can generate other statistical measures of desaturation events, such as a median, mode, weighted average, weighted median, weighted mode, or standard deviation. The generated desaturation summary data can provide a clinician greater insight into desaturation issues and thereby can improve patient care.

At block 814, a desaturation user interface can be presented. In particular, the analytics system 160 can cause presentation of a desaturation user interface. The analytics system 160 can cause presentation of the desaturation user interface on the clinician device 104. The desaturation user interface can include any of the data described above with respect to blocks 808, 810, 812, such as the desaturation summary data, the identified historical events, or metadata for one or more patients or patient devices associated with the historical events. The presented data can enable a clinician to address any of the identified desaturation issues. Example desaturation user interfaces are described in further detail above with respect to the user interfaces 600, 710, 750 of FIGS. 6, 7B, and 7C, respectively.

An example desaturation user interface can include a desaturation summary user interface, such as the user interface 600 of FIG. 6. The desaturation entry 608 can include entries for each patient from the second subset of historical events. Each entry can include a patient indicator. A patient indicator can include a patient number or string (such as “2145293587”) or the patient's first or last name. Each entry can include a group indicator (such as “ICU”) for the patient that can indicate a location of the patient or patient device or some other group information. Each entry can include a desaturation summary indicator, such as a count indicator for the quantity of desaturation events for the patient. Additional desaturation summary indicators include statistical measures for the desaturation events, such as an average, median, mode, weighted average, weighted median, weighted mode, or standard deviation. Another example desaturation summary indicator can include a statistical measure of desaturation events per time unit, such as an average number of desaturation events per hour.

As shown in the process 800, the block 814 can repeat and cause the presentation of additional user interfaces. A first desaturation user interface, such as a desaturation summary user interface, can receive user input for a selection of an entry corresponding to a patient or a patient device. Thus, the analytics system 160 can cause presentation of a second desaturation user interface, such as a desaturation detail user interface. The desaturation detail user interface can include some data from the second subset of historical events. A second desaturation user interface, such as a desaturation detail user interface, can provide a clinician additional details regarding desaturation events, which can improve patient care.

A second desaturation user interface can include a visual entry for a first desaturation event for the selected patient or patient device. The visual entry can include (i) a first indicator representing a first duration of the first desaturation event, and (ii) a second indicator representing a first oxygen saturation value for the first desaturation event. The second desaturation user interface can provide a time table of desaturation events, which can be grouped into time units. The second desaturation user interface can include a visual representation of a distribution for the desaturation events for the first patient grouped by a time unit. An example visual representation can include a histogram, bar chart, pie chart, or other representation visualizing a distribution. Example second desaturation user interface(s) are described in further detail above with respect to the user interfaces 700, 710, 750 of FIGS. 7A, 7B, and 7C, respectively.

At block 816, identified or generated data can be used. In particular, the analytics system 160 can use the identified or generated data from the previous blocks 808, 810, 812. Using the identified or generated data can include generating or exporting a report including the data. A clinician can also use the identified or generated data from the previous blocks 808, 810, 812. The analytics system 160 can use the identified or generated data to adjust or set an oxygen related alarm limit for a specific device or patient. For example, if a patient's device is alarming too often, which can be indicated by the generated summary data, then the analytics system 160 can adjust an alarm limit to reduce the number of alarms. Likewise, a clinician viewing the summary data or events of the desaturation user interfaces, such as the user interfaces 600, 710, 750 described above with respect to FIGS. 6, 7B, and 7C, can identify that a patient's device is alarming too often and can adjust an alarm limit accordingly.

The analytics system 160 or a clinician can compare alarm events with generated distributions of desaturation events, such as histograms (see FIG. 7C), to determine whether an alarm is a true alarm or not. For example, more desaturation events relative to alarm events for a time period can indicate that more alarms may be needed. Conversely, fewer desaturation events relative to alarm events can indicate that there may be too many alarms. For oxygen-related physiological parameters, the analytics system 160 can include logic to determine that where desaturation events occur at a rate above a particular threshold, the conditions for those events should not trigger further alarms (for example, a particular patient may consistently have low oxygen levels that may not necessarily necessitate alarms). Conversely, a clinician can compare alarm events with generated distributions of desaturation events and determine that there are not enough alarms. For example, a clinician can view the distributions of the desaturation events shown in FIG. 7C and determine that the alarm limits for oxygen saturation should be adjusted to generate additional alarms for the desaturation events.

### VI. Example Trend Analytics User Interfaces

A trend analytics user interface can advantageously improve patient care. Physiological parameters for a patient can have significant clinical importance. A trend analytics user interface can enable a clinician to identify or review historical events or historical summary data associated with a patient. Once historical events or historical summary data associated with a patient have been identified or reviewed, prophylactic steps can be taken to improve patient care. In response to identification of one or more clinical situations, a clinician or the analytics system 160 of FIG. 1 can proceed with one or more corrective measures, such as setting or updating an alarm for one or more patient devices to cause an alert upon detection of a corresponding situation. Further, in response to identification of one or more clinical situations, any number of other corrective measures can be put into place in a clinical setting, such as adjusting or increasing the clinical staff or other resources in a physical location where the corresponding situations have been detected. Accordingly, a trend analytics user interface can advantageously cause patient care to be improved.

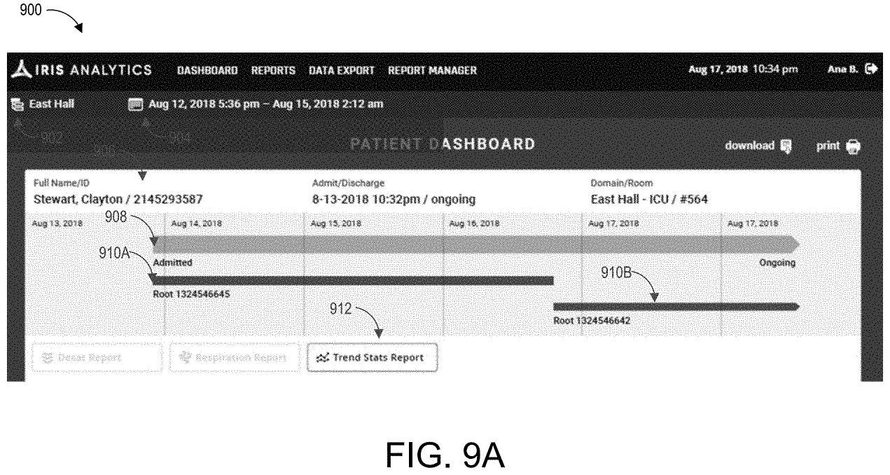

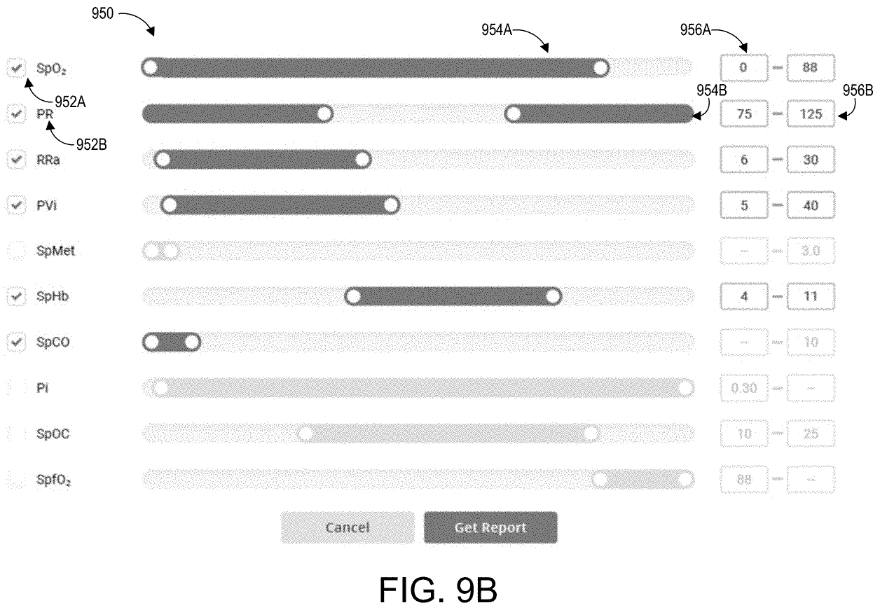

FIGS. 9A and 9B depict additional analytics user interfaces 900, 950 that may be presented by the clinician devices 104 described above. One or both of the analytics user interfaces 900, 950 of FIGS. 9A and 9B, respectively, can be presented by the clinician devices 104. The analytics user interfaces 900, 950 of FIGS. 9A and 9B, respectively, can be presented together or separately. The analytics user interface 950 of FIG. 9B can be a trend analysis parameter selection user interface. In some configurations, the analytics user interface 900 of FIG. 9A can be shown above the analytics user interface 950 of FIG. 9B. Turning to FIG. 9A, the analytics user interface 900 can include a group user interface element 902 and a time range user interface element 904. The selected group or time range from the respective elements 902, 904 can be based on user input from the user interfaces 200, 300 described above from FIGS. 2 and 3, respectively. Subsequent user interfaces, such as trend analytics user interface(s), can use the selected group or time range for analytics purposes. However, in some embodiments, a selected group or time range corresponding to the respective elements 902, 904 do not have to be used by subsequent user interfaces. The analytics user interface 900 can include a timeline user interface element 906.

The timeline user interface element 906 can be part of a patient dashboard and can indicate a selected patient. As shown, the selected patient here is “Stewart, Clayton.” The timeline user interface element 906 can present a timeline visualization for the patient. The timeline user interface element 906 can include a first timeline element 908 and one or more additional timeline elements 910A, 910B. The first timeline element 908 can indicate admit or discharge information for a patient. Admitting or discharging a patient can refer to associating or disassociating the patient with or from a patient device, which can be different than admitting or discharging a patient to or from a healthcare facility (but may be part of those processes). Following an admission of a patient to a patient device, real or near-real time data from the patient device for the particular patient may be available. As used herein, “discharge,” in addition to having its ordinary meaning, can refer to removal of a patient from one or more patient devices.

Following a discharge of a patient from a particular patient device, additional real or near-real time data from the patient device for the particular patient may not be available. As shown, the first timeline element 908 or the timeline user interface element 906 can indicate that the particular patient was admitted at a particular date/time, such as Aug. 13, 2018 at 10:32 PM. The first timeline element 908 or the timeline user interface element 906 can indicate that the particular patient was discharged or has not been discharged and is currently admitted, as indicated by the “ongoing” text shown. The one or more additional timeline elements 910A, 910B can indicate respective patient devices associated with the patient on a timeline, such as the first timeline element 910A for a first device (here “Root 1324546645”) and the second timeline element 910B for a second device (here “Root 1324546642”).

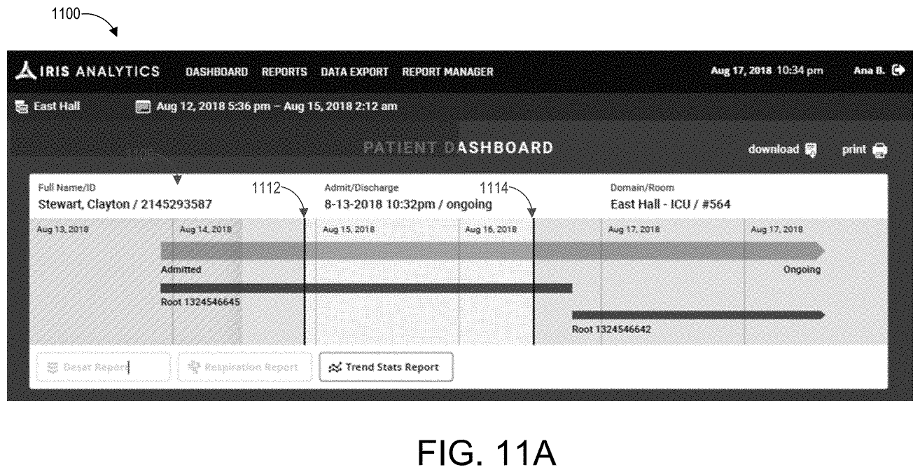

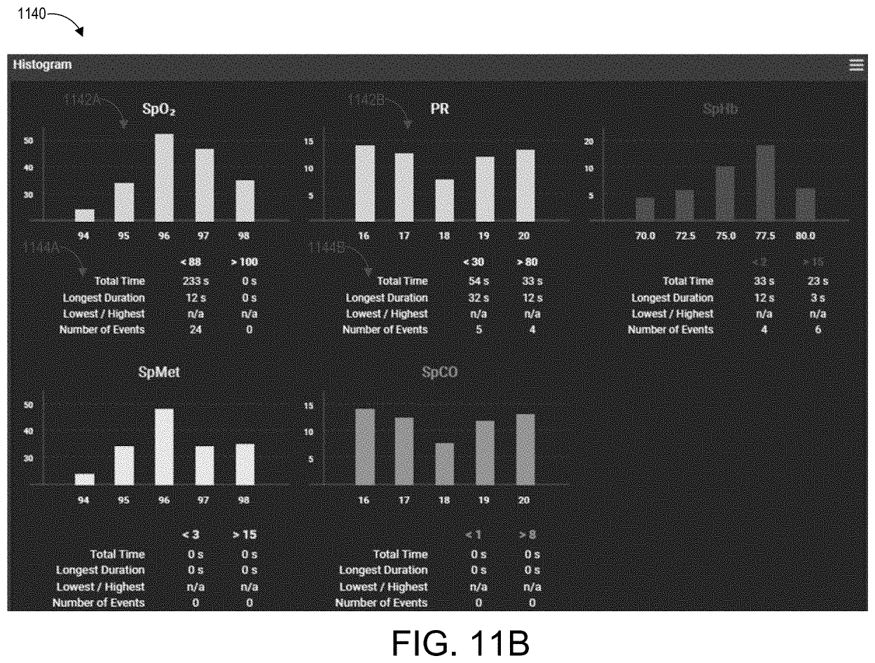

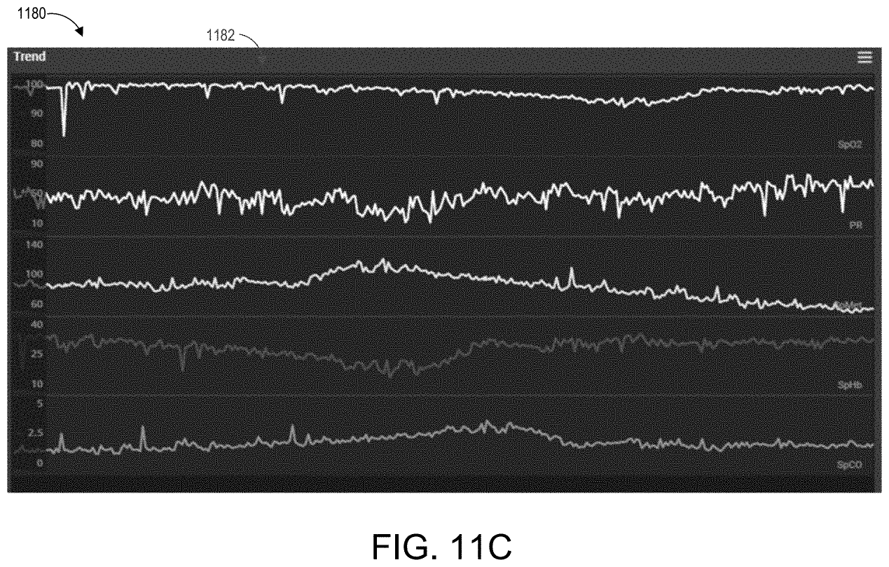

Turning to FIG. 9B, the trend analysis parameter selection user interface 950 can be used to receive parameter(s) for trend analysis user interfaces, such as a trend statistics report, which is described in further detail below with respect to FIGS. 11A-11B. The trend analysis parameter selection user interface 950 can include elements 952A, 954A, 956A, 952B, 954B, 956B associated with one or more physiological parameters. The elements 952A, 954A, 956A, 952B, 954B, 956B can enable a clinician to provide user input that can be used as criteria to generate trend analytics summary data. As shown, example physiological parameters can include oxygen saturation (SpO2), pulse rate (PR), acoustic respiration rate (RRa®), pulse variability index (PVi®), methemoglobin (SpMet®), total hemoglobin (SpHb®), carboxyhemoglobin (SpCO), perfusion index (Pi), oxygen content (SpOC™), and fractional arterial oxygen saturation (SpfO2™). A clinician can define a physiological profile for reviewing trending analytics by selecting one or more of the parameters associated with the elements 952A, 954A, 956A, 952B, 954B, 956B.

The trend analysis parameter selection user interface 950 can be an interactive user interface. A clinician can select or deselect the selector user interface elements 952A, 952B to identify one or more corresponding physiological parameters to be used in a trend analysis user interface. As shown, the selector user interface elements 952A, 952B are selected for the oxygen saturation (SpO2) and pulse rate (PR) parameters, respectively. Also as shown, other selector user interface elements may not be currently selected, such as the elements for the methemoglobin (SpMet®), perfusion index (Pi), oxygen content (SpOC™), and fractional arterial oxygen saturation (SpfO2™) parameters.

A clinician can select first threshold user interface elements 954A, 954B to identify one or more thresholds for corresponding physiological parameters to be used in a trend analysis user interface. The second threshold user interface elements 956A, 956B can reflect the selections of the first threshold user interface elements 954A, 954B. As shown, endpoints of the first threshold user interface element 954A can correspond to the indicator values of the second threshold user interface elements 956A, which is 0% and 88% oxygen saturation (SpO2) in this example, such that summary data can be presented where the parameter is between the thresholds (here, where the oxygen saturation (SpO2) is between 0% and 88%). As shown, endpoints of the other first threshold user interface element 954B can correspond to the indicator values of the other second threshold user interface element 956B, which is 75 and 125 pulse rate (PR) in this example, such that summary data can be presented where the parameter is: (i) above or below a first threshold (here, where the pulse rate is below 75) or (ii) above or below a second threshold (here, where the pulse rate is above 125). A clinician can modify the thresholds by entering numerical values into the second threshold user interface elements 956A, 956B.

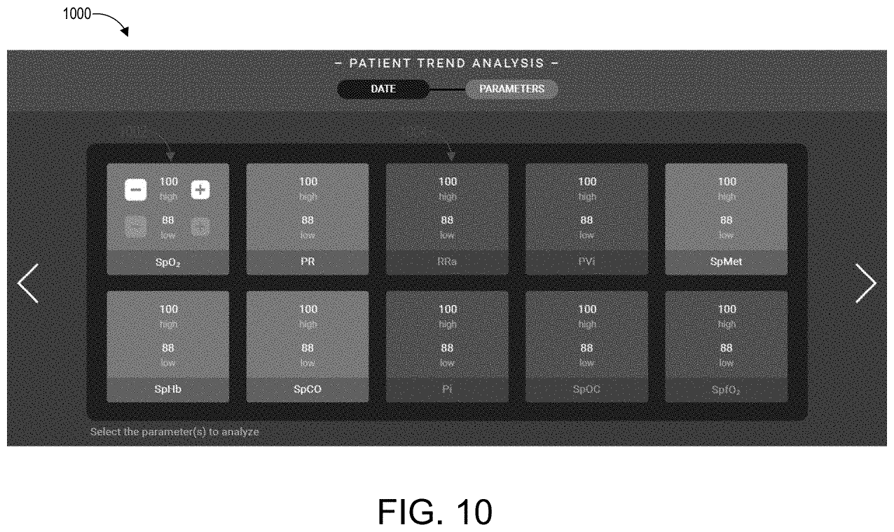

FIG. 10 depicts another example trend analysis parameter selection user interface 1000 that may be presented by the clinician devices 104 described above. The trend analysis parameter selection user interface 1000 of FIG. 10 can be similar to the trend analysis parameter selection user interface 950 of FIG. 9B. Using the trend analysis parameter selection user interface 1000, a clinician can select one or more physiological parameters to be used in a trend analysis user interface. The trend analysis parameter selection user interface 1000 can include a first parameter user interface element 1002 for a first physiological parameter (here, oxygen saturation (SpO2)) and a second parameter user interface element 1004 for a second physiological parameter (here, acoustic respiration rate (RRa®)). The first parameter user interface element 1002 is shown as selected and the second parameter user interface element 1004 is shown as not selected. A clinician can provide user input to deselect the first parameter user interface element 1002. Conversely, a clinician can provide user input to select the second parameter user interface element 1004. The first parameter user interface element 1002 can receive user input to adjust one or more thresholds associated with the first physiological parameter. Example thresholds include a high threshold (here, shown as 100) and a low threshold (here shown as 88).

FIGS. 11A through 11C depict additional example analytics user interfaces 1100, 1140, 1180 that may be presented by the clinician devices 104 described above. One or more of the analytics user interfaces 1100, 1140, 1180 of FIGS. 11A, 11B, and 11C, respectively, can be presented by the clinician devices 104. Two or more of the analytics user interfaces 1100, 1140, 1180 of FIGS. 11A, 11B, and 11C, respectively, can be presented together or separately. The analytics user interfaces 1140, 1180 of FIGS. 11B and 11C, respectively, can be trend analysis user interfaces. In some configurations, the analytics user interface 1100 of FIG. 11A can be shown above the analytics user interface 1140 of FIG. 11B, or the analytics user interface 1140 of FIG. 11B can be shown above the analytics user interface 1180 of FIG. 11C.

Turning to FIG. 11A, the analytics user interface 1100 can include a timeline user interface element 1106. The analytics user interface 1100 of FIG. 11A can be similar to the analytics user interface 900 of FIG. 9A. In particular, the timeline user interface element 1106 of FIG. 11A can be similar to the timeline user interface element 906 of FIG. 9A. However, in contrast to the timeline user interface element 906 of FIG. 9A, the timeline user interface element 1106 of FIG. 11A can include a start time selector 1112 and an end time selector 1114. A clinician can adjust a time range that can be used by subsequent user interfaces by adjusting one or more of the start time selector 1112 or the end time selector 1114.

Turning to FIG. 11B, the trend analysis user interface 1140 can include a visual representation of a distribution of trending events for a particular patient. The visual representation of trending events from FIG. 7C can be based on the parameters of previous user interfaces, such as the user interfaces of FIGS. 9A, 9B, 10, and 11A described above. An example visual representation is a histogram of respective counts of trending events for a particular patient grouped by a value. The trend analysis user interface 1140 can include visual representations 1142A, 1142B for respective physiological parameters. The trend analysis user interface 1140 can include summary data 1144A, 1144B for respective physiological parameters. As shown, the first visual representation 1142A can be for a first physiological parameter (here oxygen saturation (SpO2)) and can indicate a distribution of trending events that correspond to particular physiological parameter values (here 94%, 95%, 96%, 97%, and 98%). As shown, the first summary data 1144A for the first physiological parameter can indicate a total time, a longest duration, a lowest or highest value, or a number of the trending events for respective thresholds (here oxygen saturation (SpO2) trending events that are less than 88%).

The trend analysis parameters for selected physiological parameters, which are described above with respect to FIGS. 9B and 10, can be used to identify corresponding trending events that satisfy one or more thresholds. Moreover, the trending events can be identified based on a selected patient, a selected group, or a selected time range. In the context of the first summary data 1144A for the first physiological parameter (here oxygen saturation (SpO2)), a trending event greater than a certain threshold (here 100%) may not be applicable, and, therefore, no applicable summary data for that condition may be presented. Alternatively, the second summary data 1144B for a second physiological parameter can indicate a total time, a longest duration, a lowest or highest value, or a number of the trending events for respective thresholds, including being less than a particular threshold (here less than 30) and above another threshold (here greater than 80). The visual representations 1142A, 1142B and the summary data 1144A, 1144B for respective physiological parameters are illustrative and may not correspond exactly to the thresholds or parameters shown in the user interfaces 950, 1000 of FIGS. 9B and 10, respectively.

Turning to FIG. 11C, the trend analysis user interface 1180 can include a visualization 182 that represents one or more physiological parameter values over time. As shown, the visualization 182 can include one or more time series visualizations for respective physiological parameters. The visualization 182 can be for the selected physiological parameter(s) described above with respect to FIGS. 9B and 10 or for a selected time range, such as described above with respect to FIG. 11A. Accordingly, the trend analysis user interface 1180 of FIG. 11C can give an overview of changes regarding one or more physiological parameter values over time, which can be combined with the visual representations described above with respect to the trend analysis user interface 1140 of FIG. 11B.

### VII. Example Trend Analytics Process

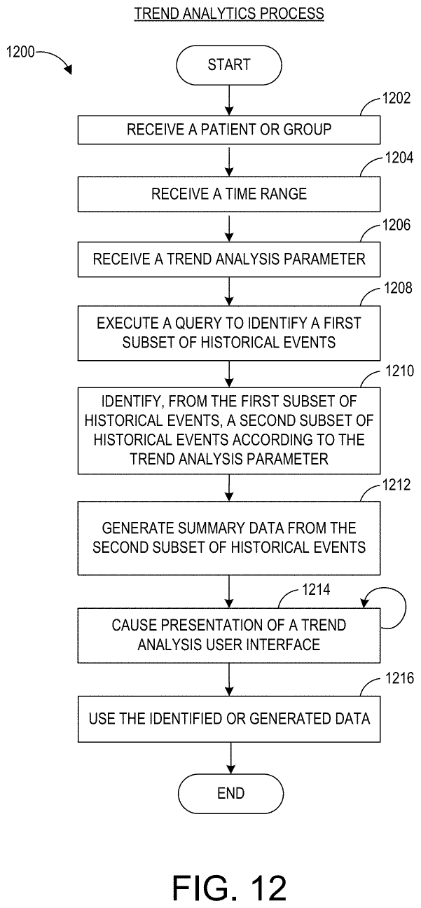

Turning to FIG. 12, an example trend analytics process 1200 is shown. The trend analytics process 1200 may be implemented by the analytics system 160. For convenience, the trend analytics process 1200 will be described in the context of the analytics system 160, although other computing systems not described herein may implement the trend analytics process 1200. The trend analytics process 1200 can advantageously provide improved patient care by enabling identification or enabling more rapid identification of patient issues enabling further actions, such as the implementation of corrective measures, as described herein.

At block 1202, a patient or group can be received. In particular, the analytics system 160 can receive a patient identifier or a group identifier. Block 1202 of FIG. 12 can be similar to block 802 of FIG. 8. A clinician can select a particular patient to generate trend analytics for the patient, and a user interface can receive the selected patient. As described above with respect to the user interface 200 of FIG. 2, a user interface can receive user input, and the user input can include a group. A selected patient or a group identifying a subset of patient devices or patients can be used to identify a subset of historical events that can be used for trend analytics. Identifying a subset of trend analytics may be advantageous because determining or presenting trend analytics for a larger set of patient devices or patients may be computationally more expensive or impractical, may result in a more cluttered user interface, or may make it slower for a clinician to access desired data.

At block 1204, a time range can be received. In particular, the analytics system 160 can receive a time range. Block 1204 of FIG. 12 can be similar to block 804 of FIG. 8. A time range can include a start time and an end time. As described above with respect to the user interfaces 300, 1100 of FIGS. 3 and 11, respectively, a user interface can receive user input, and the user input can include a time range. Similar to the use of a patient or group at block 1202, the time range can be used to identify a subset of historical events that can be used for trend analytics, such as the historical events that occurred or that includes a timestamp within the start time and end time of the time range. Again, similar to the advantages of using a patient identifier or group to identify a subset of patient devices or patients, there can be advantages to using a time range to identify a subset of historical events, such as, increasing computational efficiency or even feasibility, making a user interface less cluttered, or making it faster for a clinician to access desired data.

At block 1206, one or more trend analysis parameters can be received. In particular, the analytics system 160 can receive a trend analysis parameter. Block 1206 of FIG. 12 can be similar to block 806 of FIG. 8. The trend analysis parameter(s) can be used to identify a subset of historical events, which can be used for analytics purposes. As described above with respect to FIGS. 9B and 10, an example trend analysis parameter can include criteria, such as one or more thresholds, for one or more physiological parameters (such as oxygen saturation (SpO2), pulse rate (PR), acoustic respiration rate (RRa®), pulse variability index (PVi®), methemoglobin (SpMet®), total hemoglobin (SpHb®), carboxyhemoglobin (SpCO), perfusion index (Pi), oxygen content (SpOC™), and fractional arterial oxygen saturation (SpfO2™)). Example trend analysis parameters can include: less than a first threshold, greater than a second threshold, between a first threshold and a second threshold, or the like.

At block 1208, a query can be executed to identify a subset of historical events. In particular, the analytics system 160 can execute a query to identify a first subset of historical events. Block 1208 of FIG. 12 can be similar to block 808 of FIG. 8. As described above with respect to FIGS. 1A and 1C, the data storage 120 can be configured to store historical events. Each historical event in the data storage 120 can include a physiological parameter value for a patient at a timestamp. The query can include input parameters, such as a patient identifier, a group, a start time, or an end time. Executing the query can include identifying, from the historical events in the data storage 120, a first subset of historical events from the patient or group and within the start time and the end time. The query can include input parameters, such as a patient identifier (for example, “2145293587”), or a group, and a start time (such as Aug. 14, 2018 at 5:36 PM) and an end time (such as Aug. 16, 2018 at 2:12 AM). Executing the query can result in identifying historical events for particular physiological parameters (such as one or more values for oxygen saturation, pulse rate, acoustic respiration rate, pulse variability index, methemoglobin, total hemoglobin, carboxyhemoglobin, perfusion index, oxygen content, or a fractional arterial oxygen saturation for a patient at respective timestamps) that were captured by a device for a patient within the start time and the end time. This can enable the clinician to advantageously view historical events or summary data of those events to determine clinical issues.

At block 1210, a second subset of historical events can be identified from the first subset of historical events. In particular, the analytics system 160 can identify the second subset of historical events from the first subset of historical events. Block 1210 of FIG. 12 can be similar to block 810 of FIG. 8. The analytics system 160 can identify the second subset of historical events using the one or more trend analysis parameters. As described above respect to block 1206, a trend analysis parameter can include a threshold for a physiological parameter. The analytics system 160 can identify, for a particular patient, respective physiological parameter values and timestamps that satisfy the trend analysis parameter. Moreover, the analytics system 160 can identify, from the first subset of historical events, those historical events that satisfy the trend analysis parameter.

In some embodiments, blocks 1208 and 1210 can be combined. In particular, instead of identifying the first subset of historical events at block 1208 and the second subset of historical events at block 1210, a single subset of historical events can be determined together. The analytics system 160 can execute a query to determine a single subset of historical events. The query can include any of the parameters, such as a patient identifier, a group, a time range, or a trend analysis parameter, from the previous blocks 1202, 1204, 1206. The query can include one or more trend analysis parameters to determine the subset of historical events, and the query can be in a format configured for a database, such as in a SQL format or an object-oriented database query format.

At block 1212, summary data can be generated from the second subset of historical events. In particular, the analytics system 160 can generate summary data from the second subset of historical events. Block 1212 of FIG. 12 can be similar to block 812 of FIG. 8. The analytics system 160 can calculate a quantity of trending events from the second subset of historical events. As described above with respect to block 810, the second subset of historical events can reflect the trend analysis parameter(s), such as by indicating those historical event(s) that correspond to one or more thresholds, such as being less than a threshold, greater than a threshold, or between a first threshold and a second threshold, thereby identifying a trending event. The analytics system 160 can quantify the identified trending events, such as by calculating statistical summary data that can include a total number of trending events, a total time of the trending events, a longest in time trending event, a highest value trending event, a lowest value trending event, or an average number of trending events per time unit, which can be for each respective threshold, such as those trending events below a first threshold or above a second threshold. The analytics system 160 can generate other statistical measures of trending events, such as a median, mode, weighted average, weighted median, weighted mode, or standard deviation. The generated summary data can provide a clinician greater insight into clinical issues and thereby can improve patient care.

At block 1214, a trending analysis user interface can be presented. In particular, the analytics system 160 can cause presentation of a trending analysis user interface. Block 1214 of FIG. 12 can be similar to block 814 of FIG. 8. The analytics system 160 can cause presentation of the trending analysis user interface on the clinician device 104. The trending analysis user interface can include any of the data described above with respect to blocks 1208, 1210, 1212, such as the summary data, the identified historical events, or metadata for one or more patients or patient devices associated with the historical events. The presented data can enable a clinician to address any of the identified clinical issues. Example trending analysis user interfaces are described in further detail above with respect to the user interfaces 1140, 1180 of FIGS. 11B and 11C respectively.

As shown in the process 1200, the block 1214 can repeat and cause the presentation of additional user interfaces. A first trending analysis user interface can receive user input, which can cause the analytics system 160 to cause presentation of a second trending analysis user interface based on the received user input. Example user input can include an additional or a change to a trending analysis parameter, or a new patient, group, or time range, or some combination thereof, which can cause an updated trending analysis user interface to be presented.

At block 1216, identified or generated data can be used. In particular, the analytics system 160 can use the identified or generated data from the previous blocks 1208, 1210, 1212. Using the identified or generated data can include generating or exporting a report including the data. A clinician can also use the identified or generated data from the previous blocks 1208, 1210, 1212. Block 1216 of FIG. 12 can be similar to block 816 of FIG. 8. The analytics system 160 can use the identified or generated data to adjust or set an alarm limit for a specific patient. For example, if the patient's device is alarming too often, which can be indicated by the generated summary data, then the analytics system 160 can adjust an alarm limit to reduce the number of alarms. Likewise, a clinician viewing the summary data of the trending analysis user interfaces, such as the user interfaces 1140, 1180 described above with respect to FIGS. 11B and 11C, can identify that a patient's device is alarming too often and can adjust an alarm limit accordingly.

The analytics system 160 or a clinician can compare alarm events with generated distributions of trending events, such as histograms, to determine whether an alarm is a true alarm or not. For particular physiological parameters, the analytics system 160 can include logic to determine that where particular events occur at a rate above a particular threshold that the conditions for those events should not trigger further alarms. Conversely, the analytics system 160 or a clinician can compare alarm events with generated distributions of trending events and determine that there are not enough alarms. The analytics system 160 or a clinician can add additional alarms for a particular patient. For particular physiological parameters, the analytics system 160 can include logic to determine that where particular events occur at a rate above a particular threshold and there are not any or enough alarms above another threshold, then the conditions for those events should trigger further alarms.

### VIII. Additional Example Analytics User Interfaces

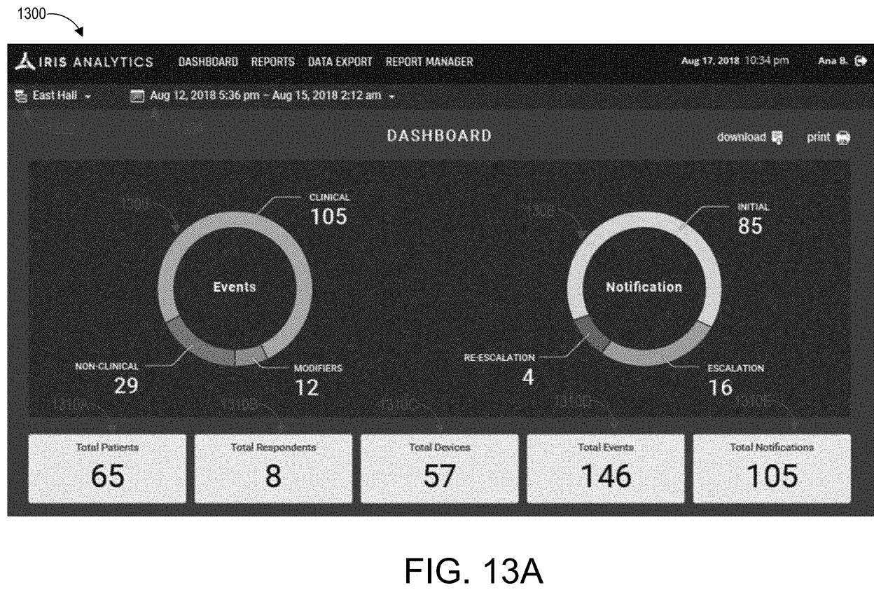

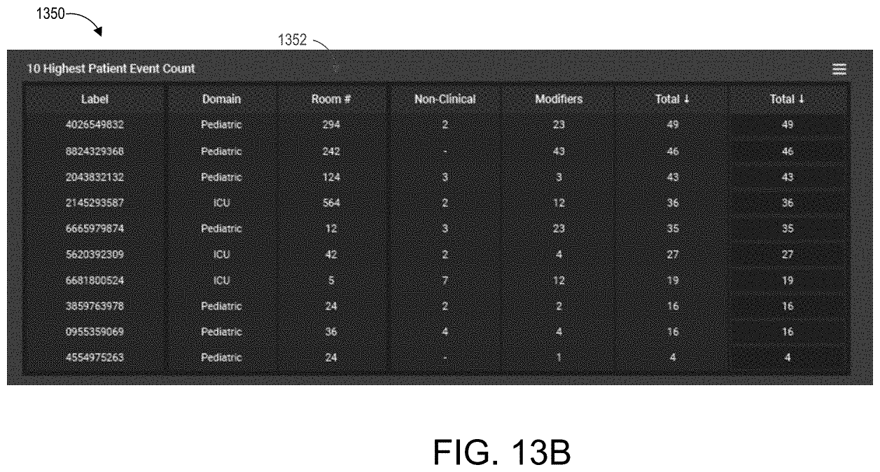

FIGS. 13A and 13B depict example dashboard analytics user interfaces 1300, 1350 that may be presented by the clinician devices 104 described above. One or both of the dashboard analytics user interfaces 1300, 1350 of FIGS. 13A and 13B, respectively, can be presented by the clinician devices 104. The dashboard analytics user interfaces 1300, 1350 of FIGS. 13A and 13B, respectively, can be presented together or separately. In some configurations, the dashboard analytics user interface 1300 of FIG. 13A can be shown above the dashboard analytics user interface 1350 of FIG. 13B.

Turning to FIG. 13A, the dashboard analytics user interface 1300 can be similar to the analytics user interface 900 of FIG. 9A. The dashboard analytics user interface 1300 can include a group user interface element 1302, which can be similar to the group user interface element 902 of FIG. 9A. The dashboard analytics user interface 1300 can include a time range user interface element 1304, which can be similar to the time range user interface element 904 of FIG. 9A. The dashboard analytics user interface 1300 can use the selected group or time range from the respective elements 1302, 1304.

The example dashboard analytics user interface 1300 show can include a first visual event summary indicator 1306 or a second visual event summary indicator 1308. The first visual event summary indicator 1306 can present a visual summary indicating a respective number of one or more clinical events (here 105), non-clinical events (here 29), or modifier events (here 12). The second visual event summary indicator 1308 can present a visual summary indicating a respective number of one or more notifications, such as initial notifications (here 85), escalation notifications (here 16), or re-escalation notifications (4). The first visual event summary indicator 1306 can include a respective number of clinical events, non-clinical events, or modifier events. The second visual event summary indicator 1308 can be based on an input parameter, such as a selected group or a time range. In particular, if an input parameter (such as a group or time range) changes, then the first visual event summary indicator 1306 or the second visual event summary indicator 1308 can dynamically update and present updated indicators.

The dashboard analytics user interface 1300 can include one or more historical metadata indicators 1310A, 1310B, 1310C, 1310D, 1310E. The first historical metadata indicator 1310A can indicate a number of patients. The second historical metadata indicator 1310B can indicate a number of respondents, such as clinicians that are assigned to respond in a clinical setting. The third historical metadata indicator 1310C can indicate a number of patient devices. The fourth historical metadata indicator 1310D can indicate a number of events. The fifth historical metadata indicator 1310E can indicate a number of notifications. In particular, the historical metadata indicators 1310A, 1310B, 1310C, 1310D, 1310E can indicate a respective number of patients, number of respondents, number of patient devices, number of events, or number of notifications that are associated with a group or time range, such as patients, respondents, or patient devices within a group, or events or notifications that originate from a group. Similar to the visual event summary indicators 1306, 1308 that can be based on an input parameter, such as a selected group or a time range, the historical metadata indicators 1310A, 1310B, 1310C, 1310D, 1310E can be based on an input parameter. Thus, if an input parameter, such as a group or time range changes, then the historical metadata indicators 1310A, 1310B, 1310C, 1310D, 1310E can update accordingly.

Turning to FIG. 13B, the example dashboard analytics user interface 1350 can include the summary analytics 1352 that can indicate one or more summary data entries. The summary analytics 1352 can indicate a statistical summary of patient events, such as a highest number of patient events (here the “10 Highest Patient Event Count”). As shown, an entry can include an identifier (such as the “Label” that can be a patient identifier), a group (here a “Domain”), a number for one or more events (such as non-clinical or modifier events), and a total number of events for each patient. Accordingly, a clinician can identify those patients with the highest number of events, where the patient is located, or other information that can be used to improve patient care.

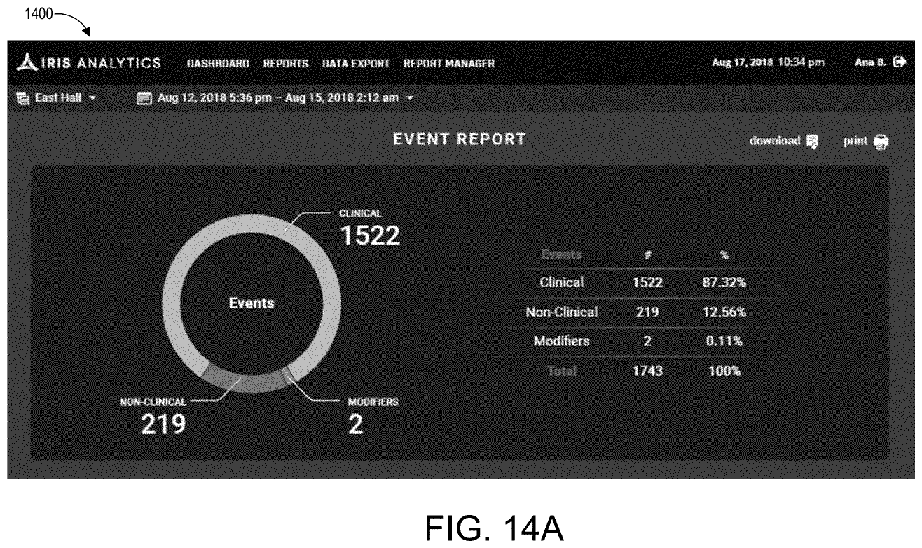

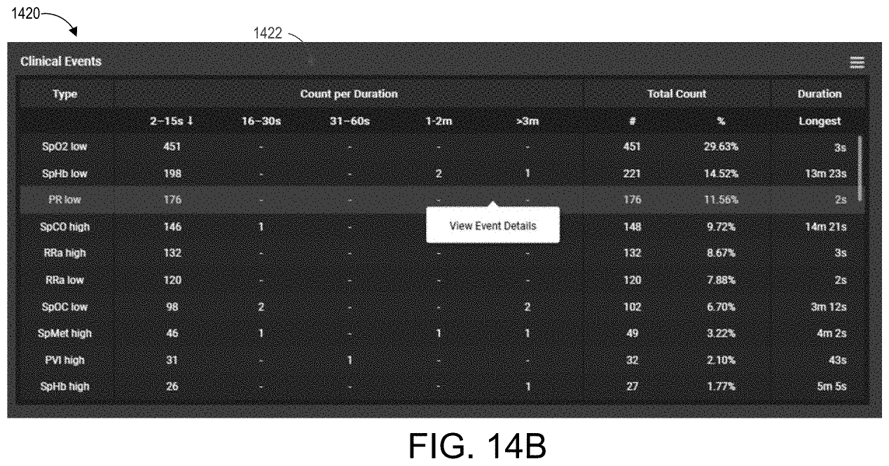

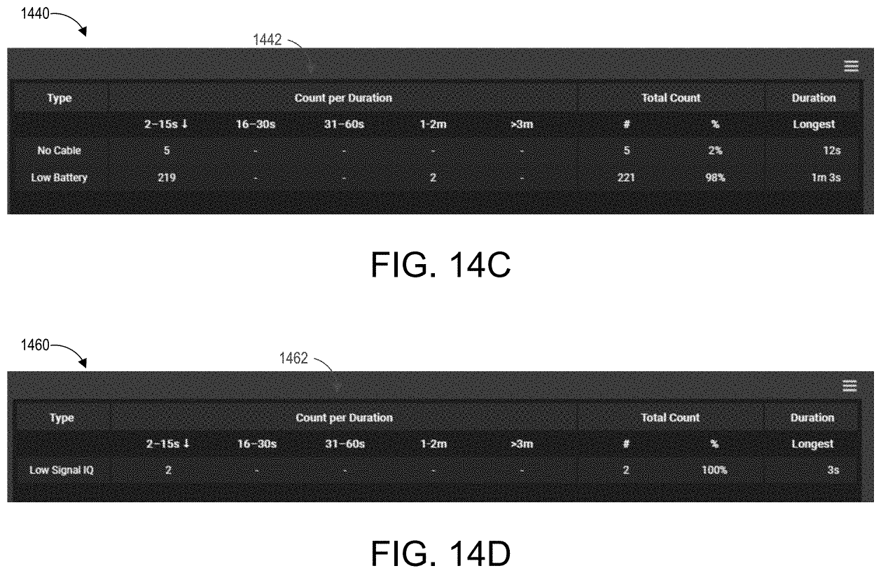

FIGS. 14A, 14B, 14C, and 14D depict additional example analytics user interfaces 1400, 1420, 1440, 1460 that may be presented by the clinician devices 104 described above. The analytics user interfaces 1400, 1420, 1440, 1460 can be depicted together as an event report. One or more of the analytics user interfaces 1400, 1420, 1440, 1460 of FIGS. 14A, 14B, 14C, and 14D, respectively, can be presented by the clinician devices 104. Two or more of the analytics user interfaces 1400, 1420, 1440, 1460 of FIGS. 14A, 14B, 14C, and 14D, respectively, can be presented together or separately. In some configurations, the analytics user interface 1400 of FIG. 14A can be shown above the analytics user interface 1420 of FIG. 14B, the analytics user interface 1420 of FIG. 14B can be shown above the analytics user interface 1440 of FIG. 14C, or the analytics user interface 1440 of FIG. 14C can be shown above the analytics user interface 1460 of FIG. 14D. Turning to FIG. 14A, the analytics user interface 1400 can be similar to the analytics user interface 1300 of FIG. 13A, such as by including similar user interface elements or similar functionality.

Turning to FIG. 14B, the analytics user interface 1420 can include first summary analytics 1422 that can indicate one or more summary data entries. The summary analytics 1422 can indicate a statistical summary of clinical events, such as counts per duration for a type of clinical event, a total count of events per event type, a percentage of events per event type, and a longest duration event per event type. A count can refer to the number of times that a type of event occurred in the selected time window. Turning to FIG. 14C, the analytics user interface 1440 can include second summary analytics 1442, which can be similar to the first summary analytics 1422 of FIG. 14B. However, in contrast to the first summary analytics 1422 of FIG. 14B that can summarize clinical events, the second summary analytics 1442 can indicate a statistical summary of non-clinical events. Turning to FIG. 14D, the analytics user interface 1460 can include third summary analytics 1462, which can be similar to the first summary analytics 1422 of FIG. 14B. However, in contrast to the first summary analytics 1422 of FIG. 14B, the second summary analytics 1442 can indicate a statistical summary of modifier events.

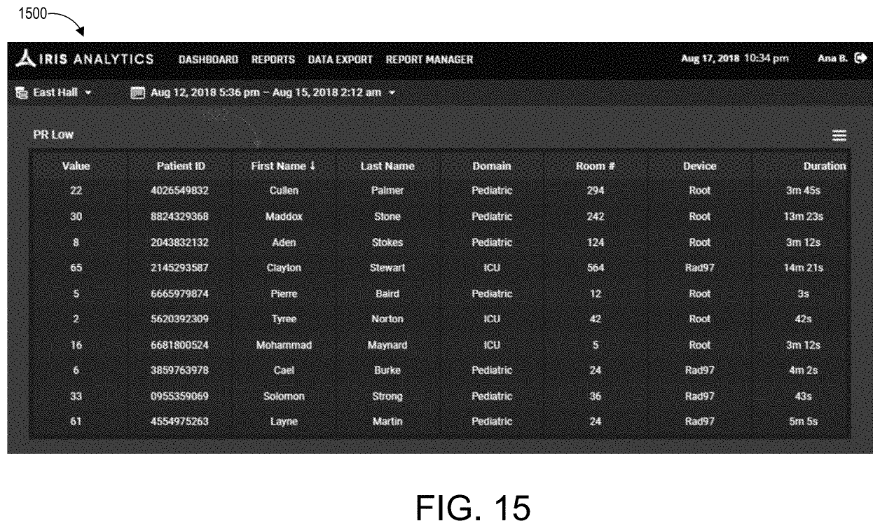

FIG. 15 depicts another example analytics user interface 1500 that may be presented by the clinician devices 104 described above. The analytics user interface 1500 can be similar to the analytics user interface 1400 of FIG. 14A or the analytics user interface 1420 of FIG. 14B, such as by including similar user interface elements or similar functionality. The analytics user interface 1500 can include low pulse rate summary analytics 1522 that can indicate one or more summary data entries. A low pulse rate event can be an example clinical event. The low pulse rate summary analytics 1522 can indicate a statistical summary of low pulse rate events, such as indicating a top predetermined number (such as 10) of patients with low pulse rate events or by indicating the lowest pulse rate events for each patient within a group. The low pulse rate summary analytics 1522 can include a low pulse rate value, one or more patient identifiers, the patient device associated with the low pulse rate event, a duration of the low pulse rate event, or other information.

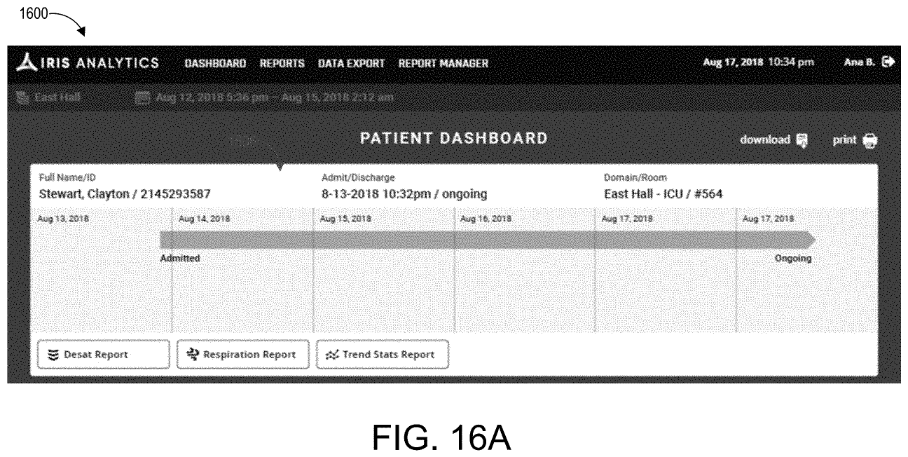

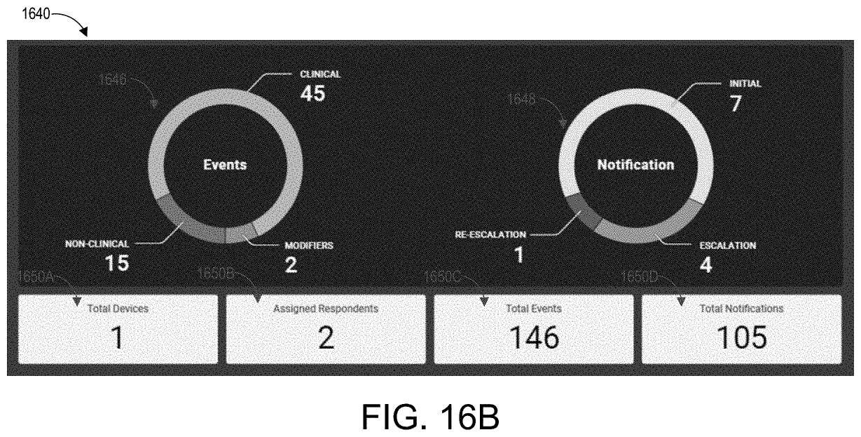

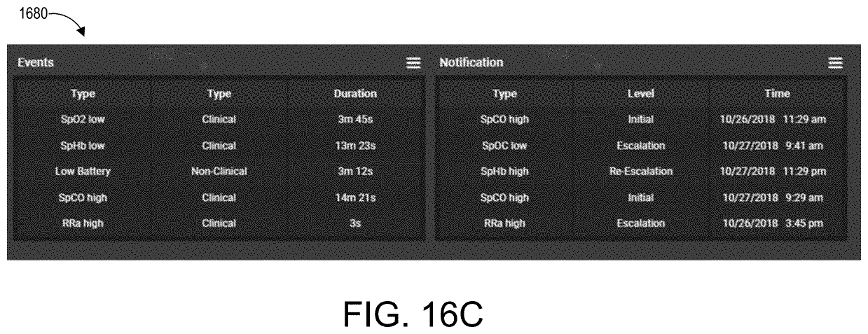

FIGS. 16A through 16C depict example patient dashboard analytics user interfaces 1600, 1640, 1680 that may be presented by the clinician devices 104 described above. One or more of the patient dashboard analytics user interfaces 1600, 1640, 1680 of FIGS. 16A, 16B, and 16C, respectively, can be presented by the clinician devices 104. Two or more of the patient dashboard analytics user interfaces 1600, 1640, 1680 of FIGS. 16A, 16B, and 16C, respectively, can be presented together or separately. In some configurations, the patient dashboard analytics user interface 1600 of FIG. 16A can be shown above the patient dashboard analytics user interface 1640 of FIG. 16B, or the patient dashboard analytics user interface 1640 of FIG. 16B can be shown above the patient dashboard analytics user interface 1680 of FIG. 16C. Turning to FIG. 16A, the patient dashboard analytics user interface 1600 can be similar to the analytics user interface 1100 of FIG. 11A, such as by including similar user interface elements or similar functionality. In particular, the patient dashboard analytics user interface 1600 can include a timeline user interface element 1606 for a particular patient, which can be similar to the timeline user interface element 1106 of FIG. 11A.

Turning to FIG. 16B, the patient dashboard analytics user interface 1640 can be similar to the dashboard analytics user interface 1300 of FIG. 13A, such as by including similar user interface elements or similar functionality. However, unlike the dashboard analytics user interface 1300 of FIG. 13A that can present data for multiple patients, the patient dashboard analytics user interface 1640 may exclusively present data for a single, particular patient. The patient dashboard analytics user interface 1640 can include a first visual event summary indicator 1646 or a second visual event summary indicator 1648, which can be similar to the first visual event summary indicator 1306 or a second visual event summary indicator 1308 of FIG. 13A; however, the first visual event summary indicator 1646 or the second visual event summary indicator 1648 can exclusively provide summary data for a particular patient instead of multiple patients. The patient dashboard analytics user interface 1640 can include one or more historical metadata indicators 1650A, 1650B, 1650C, 1650D, which can be similar to the one or more historical metadata indicators 1310A, 1310B, 1310D, 1310E of FIG. 13A; however, the one or more historical metadata indicators 1650A, 1650B, 1650C, 1650D can exclusively provide summary data for a particular patient instead of multiple patients.

Turning to FIG. 16C, the patient dashboard analytics user interface 1680 can include event analytics 1682 that can indicate one or more event data entries for a particular patient. The event analytics 1682 can include information regarding events, such as data entries that may indicate an event type (such as low oxygen or “SpO2 low”), an event category (such as clinical, non-clinical, or a modifier category), or a duration of the event. The presented event analytics 1682 can include a predetermined number of events for a patient (such as the top 5 events for a patient) based on recency, urgency, duration, or some other parameter. The patient dashboard analytics user interface 1680 can include notification analytics 1684 that can indicate one or more notification data entries for a particular patient. The notification analytics 1684 can include information regarding notifications, such as data entries that may indicate a notification type (such as high carboxyhemoglobin or SpCO®, a notification level (such as initial, escalation, or re-escalation), or a time of the notification. The presented event analytics 1684 can include a predetermined number of notifications for a patient (such as the top 5 notifications per patient) based on recency, urgency, duration, or some other factor.

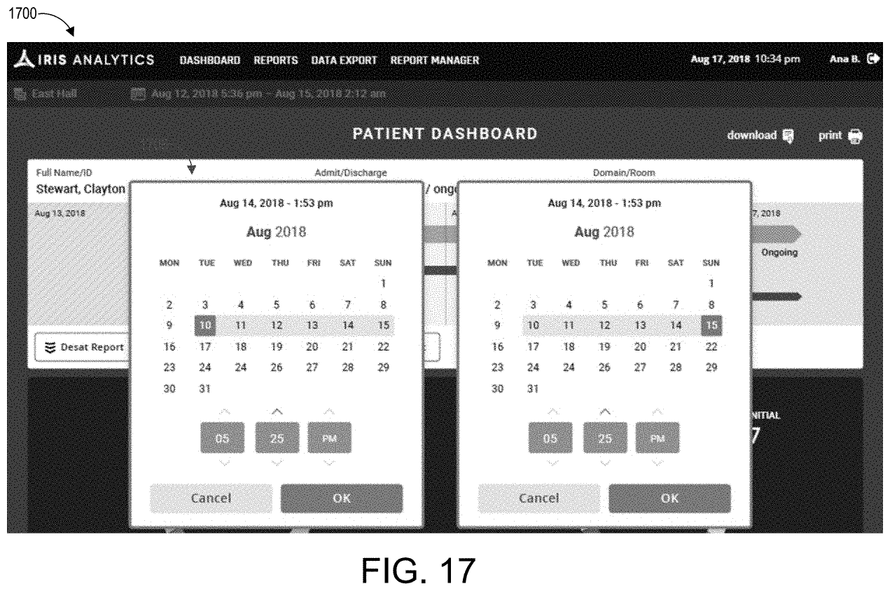

FIG. 17 depicts an example time selector user interface 1700 that may be presented by the clinician devices 104 described above. The time selector user interface 1700 can allow a clinician to configure subsequent user interfaces, such as by presenting data in subsequent user interfaces within a selected time range. As shown, the time selector user interface 1700 can include a timeline user interface element 1706 and a clinician can select a particular time range, which can include a selected start time and a selected and time. The selected time range can be used by subsequent user interfaces for the selected patient (here “Stewart, Clayton”).

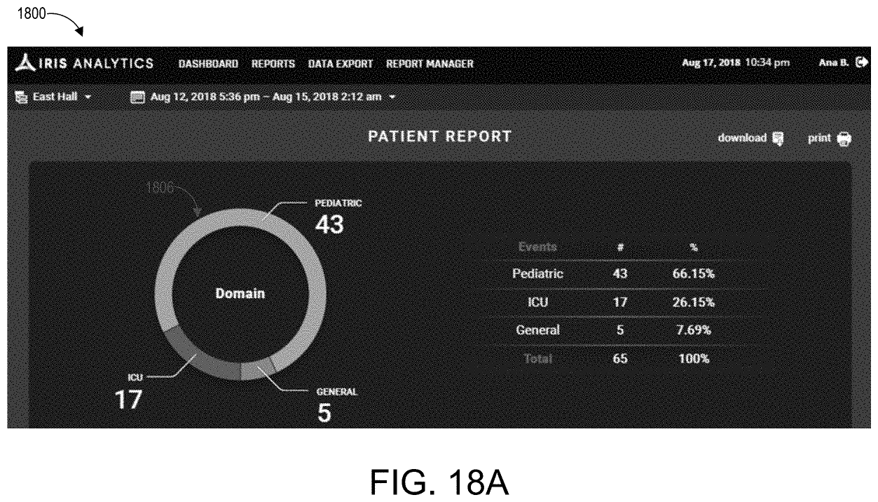

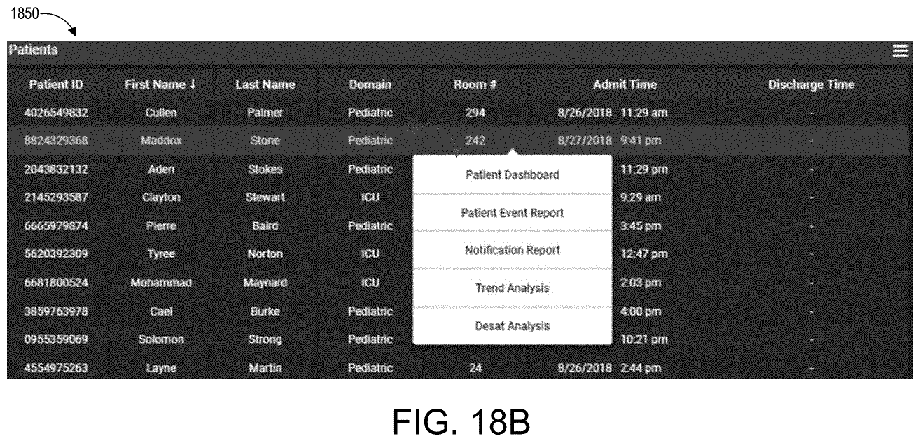

FIGS. 18A-18B depict additional example analytics user interfaces 1800, 1850 that may be presented by the clinician devices 104 described above. The analytics user interfaces 1800, 1850 may constitute a patient report together or singly, which report can present metadata regarding multiple patients. The analytics user interfaces 1800, 1850 can also enable a clinician to select one or more patients as input for additional analytics user interfaces. One or more of the analytics user interfaces 1800, 1850 of FIGS. 18A and 18B, respectively, can be presented by the clinician devices 104. The analytics user interfaces 1800, 1850 of FIGS. 18A and 18B, respectively, can be presented together or separately. In some configurations, the analytics user interface 1800 of FIG. 18A can be shown above the analytics user interface 1850 of FIG. 18B.

Turning to FIG. 18A, the analytics user interface 1800 can include a visual group summary indicator 1806. The visual group summary indicator summary indicator 1806 can present a visual summary indicating a respective number of patients associated with one or more groups (here 43 patients are located in the pediatric group, 17 patients are located in the ICU, and 5 patients are associated with a general group). Turning to FIG. 18B, the analytics user interface 1850 can include patient metadata regarding one or more patients presented as one or more data entries. Using the analytics user interface 1850, a clinician can select an entry for a particular patient to view menu options 1852. These options 1852 can provide user interface functionality to cause a presentation of one or more additional user interfaces for the selected patient. As shown, the menu options 1852 can include: a patient dashboard option that can cause the presentation of the patient dashboard user interfaces 1600, 1640, 1680 of FIGS. 16A-16C; a trend analysis option that can cause the presentation of the trend analysis user interfaces described above with respect to FIGS. 9A-9B, 10, and 11A-11C; or a desaturation analysis option that can cause the presentation of the desaturation user interfaces described above with respect to FIGS. 4-6 and 7A-7C.

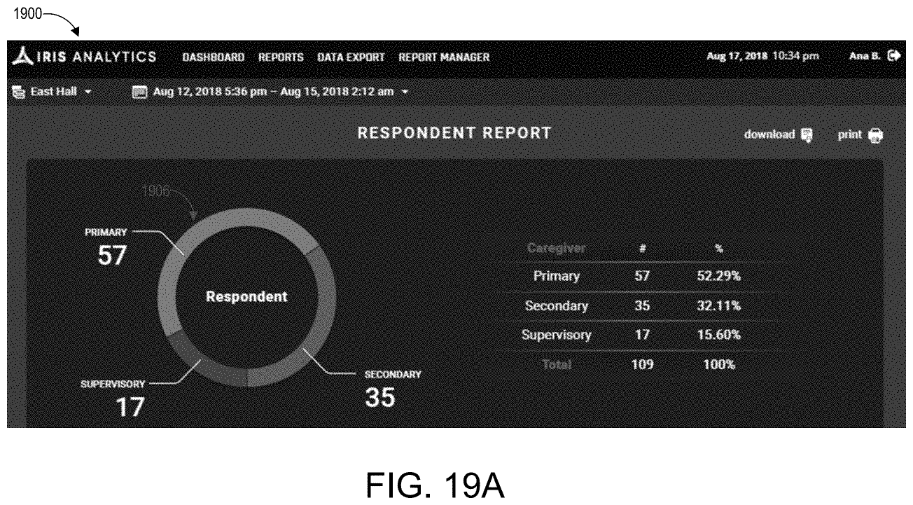

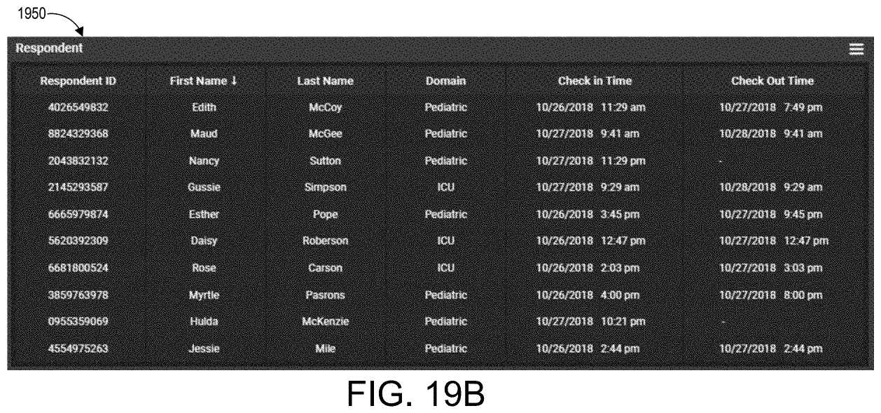

FIGS. 19A-19B depict additional example analytics user interfaces 1900, 1950 that may be presented by the clinician devices 104 described above. The analytics user interfaces 1900, 1950 may constitute a respondent report together or singly, which report can present metadata regarding multiple clinicians. One or more of the analytics user interfaces 1900, 1950 of FIGS. 19A and 19B, respectively, can be presented by the clinician devices 104. The analytics user interfaces 1900, 1950 of FIGS. 19A and 19B, respectively, can be presented together or separately. In some configurations, the analytics user interface 1900 of FIG. 19A can be shown above the analytics user interface 1950 of FIG. 19B.

Turning to FIG. 19A, the analytics user interface 1900 can include a visual respondent summary indicator 1906. The visual respondent summary indicator 1906 can present a visual summary indicating a respective number of clinician types that can be assigned to patients (here there are 57 primary clinicians, 35 secondary clinicians, and 17 supervisory clinicians). The analytics user interface 1900 can present summary data regarding clinicians that are assigned to a particular group, such as the example selected “East Hall” group shown. Turning to FIG. 19B, the analytics user interface 1950 can include clinician metadata regarding one or more clinicians presented as one or more data entries. The clinician metadata can include one or more clinician identifiers, a sub-group that the clinician is assigned to (such as the Pediatric or ICU domains), or a check-in or checkout time for the clinician.

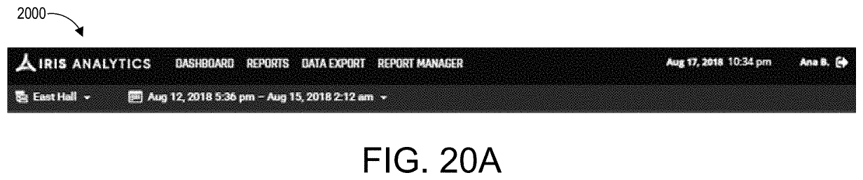

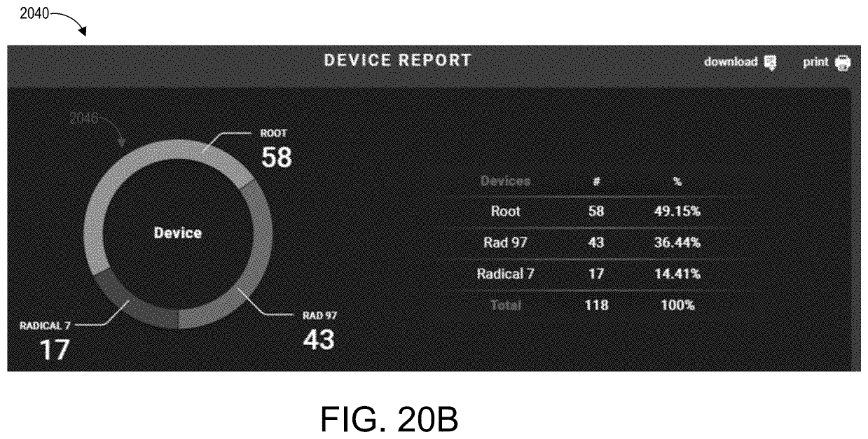

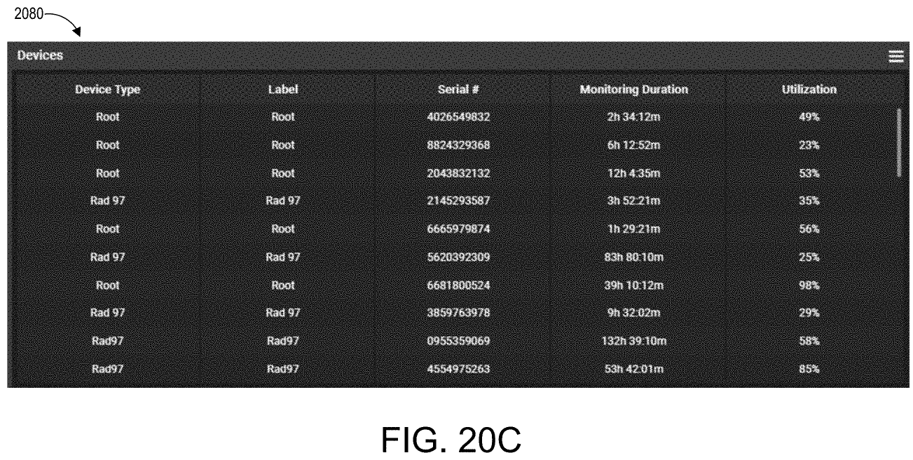

FIGS. 20A through 20C depict additional example analytics user interfaces 2000, 2040, 2080 that may be presented by the clinician devices 104 described above. One or more of the analytics user interfaces 2000, 2040, 2080 of FIGS. 20A, 20B, and 20C, respectively, can be presented by the clinician devices 104. Two or more of the analytics user interfaces 2000, 2040, 2080 of FIGS. 20A, 20B, and 20C, respectively, can be presented together or separately. The analytics user interfaces 2040, 2080 of FIGS. 20B and 20C, respectively, can be device report user interfaces. In some configurations, the analytics user interface 2000 of FIG. 20A can be shown above the analytics user interface 2040 of FIG. 20B, or the analytics user interface 2040 of FIG. 20B can be shown above the analytics user interface 2080 of FIG. 20C. Turning to FIG. 20A, the analytics user interface 2000 can be similar to the dashboard analytics user interface 1300 of FIG. 13A, such as by including similar user interface elements or similar functionality.

Turning to FIG. 20B, the analytics user interface 2000 can include a visual device summary indicator 2046. The visual device summary indicator 2046 can present a visual summary indicating a respective number of patient device types that can be assigned to patients (here there are 58 “Root” devices, 43 “Rad 97” devices, and 17 “Radical 7” devices). The analytics user interface 2040 can present summary data regarding patient devices that are assigned to a particular group, such as the selected “East Hall” group of FIG. 20A. Turning to FIG. 20C, the analytics user interface 2080 can include patient device metadata regarding one or more patient devices presented as one or more data entries. Similar to the analytics user interface 2000 of FIG. 20B, the analytics user interface 2080 of FIG. 20C can present summary data based on an input parameter, such as a selected group or time range. The patient device metadata can include one or more device types, patient device identifiers (such as a label, a device name, or a serial number), a monitoring duration, or a utilization percentage. The utilization percentage can indicate a percentage that patient devices utilize. An example calculation for utilization percentage can include taking the monitoring time of the patient device divided by a number of connected sensors to the patient device and optionally further divided by a selected time range.

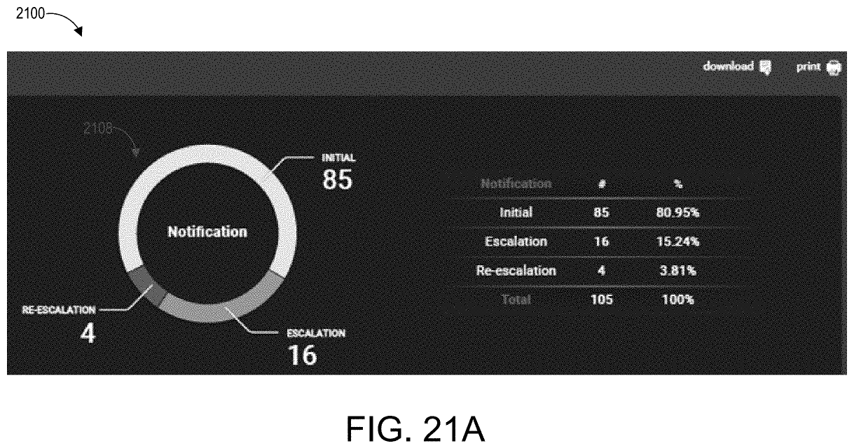

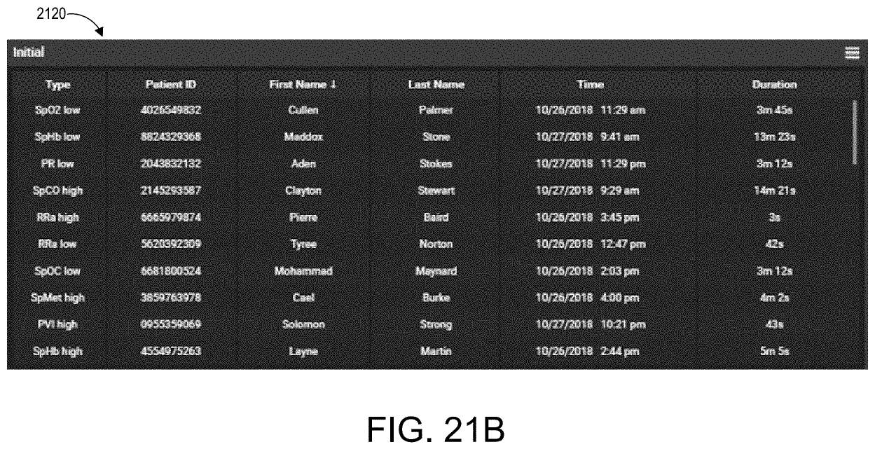

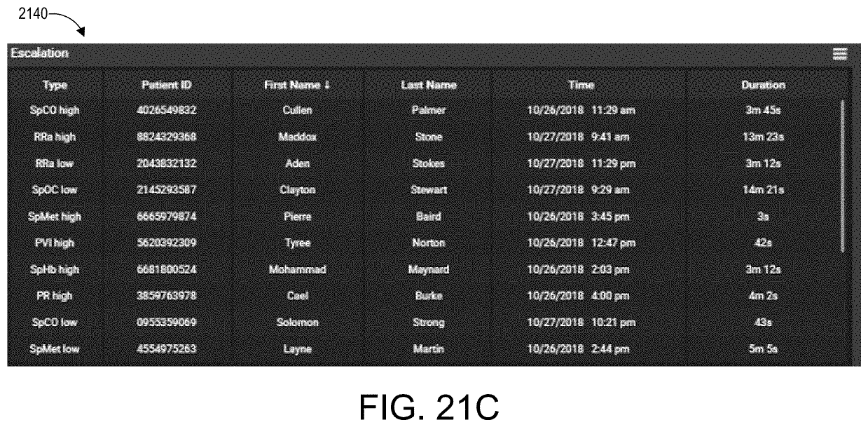

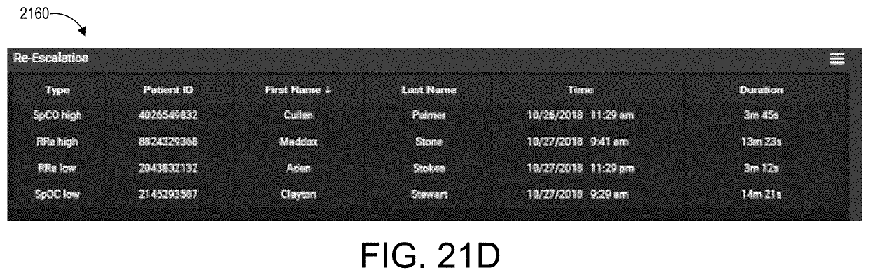

FIGS. 21A through 21D depict additional example analytics user interfaces 2100, 2120, 2140, 2160 that may be presented by the clinician devices 104 described above. The analytics user interfaces 2100, 2120, 2140, 2160 can be notification report user interfaces. One or more of the analytics user interfaces 2100, 2120, 2140, 2160 of FIGS. 21A, 21B, 21C, and 21D, respectively, can be presented by the clinician devices 104. Two or more of the analytics user interfaces 2100, 2120, 2140, 2160 of FIGS. 21A, 21B, 21C, and 21D, respectively, can be presented together or separately. In some configurations, the analytics user interface 2000 of FIG. 20A can be shown above the analytics user interface 2100 of FIG. 21A, the analytics user interface 2100 of FIG. 21A can be shown above the analytics user interface 2120 of FIG. 21B, the analytics user interface 2120 of FIG. 21B can be shown above the analytics user interface 2140 of FIG. 21C, or the analytics user interface 2140 of FIG. 21C can be shown above the analytics user interface 2160 of FIG. 21D.

Turning to FIG. 21A, the analytics user interface 2100 can include a visual notification summary indicator 2109. The visual notification summary indicator 2109 can be similar to the second visual event summary indicator 1308 of FIG. 13A, such as by presenting a visual summary indicating a respective number of one or more notifications. Turning to FIGS. 21B, 21C, and 21D, the analytics user interfaces 2120, 2140, 2160 can each present notification data in the form of one or more data entries; however, the analytics user interfaces 2120, 2140, 2160 may each present a different type of notification, such as, initial notifications, escalation notifications, and re-escalation notifications, respectively. The one or more data entries can indicate a notification type (such as “SpO2 low” or “SpCO high”), one or more identifiers, a time of the notification, and a duration of the notification. The analytics user interfaces 2120, 2140, 2160 can be based on an input parameter, such as a selected group or a time range. Thus, if an input parameter, such as a group or time range changes, then the analytics user interfaces 2120, 2140, 2160 can update accordingly.

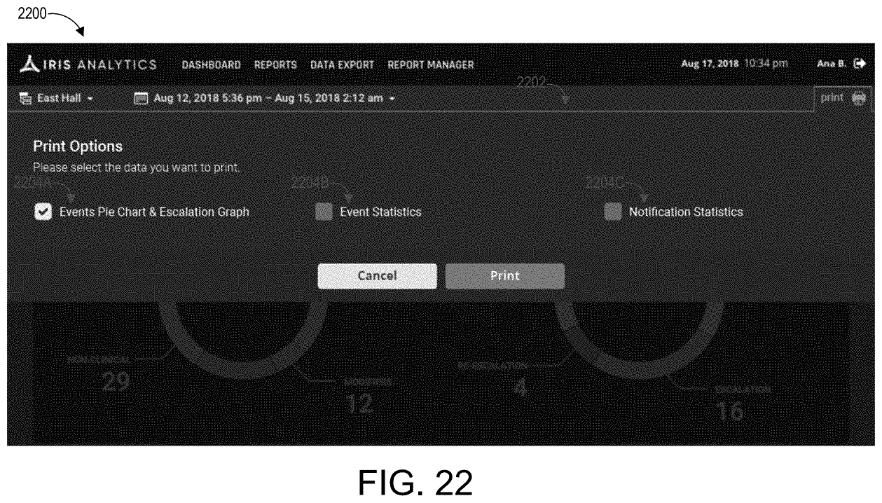

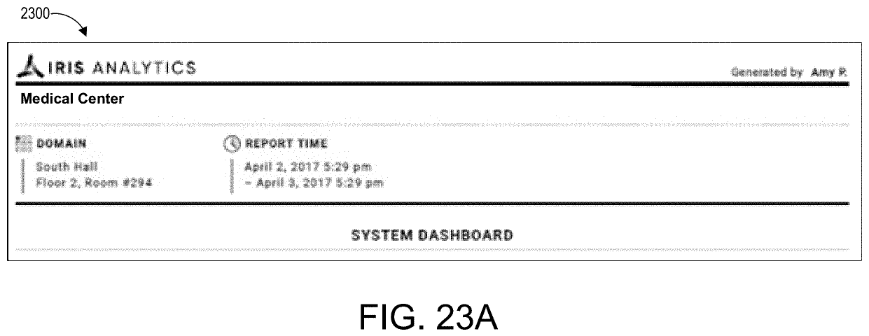

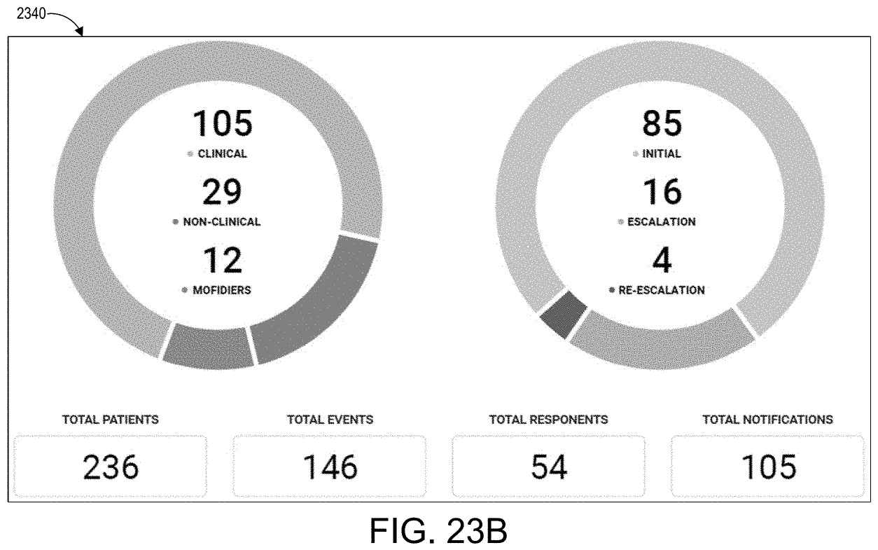

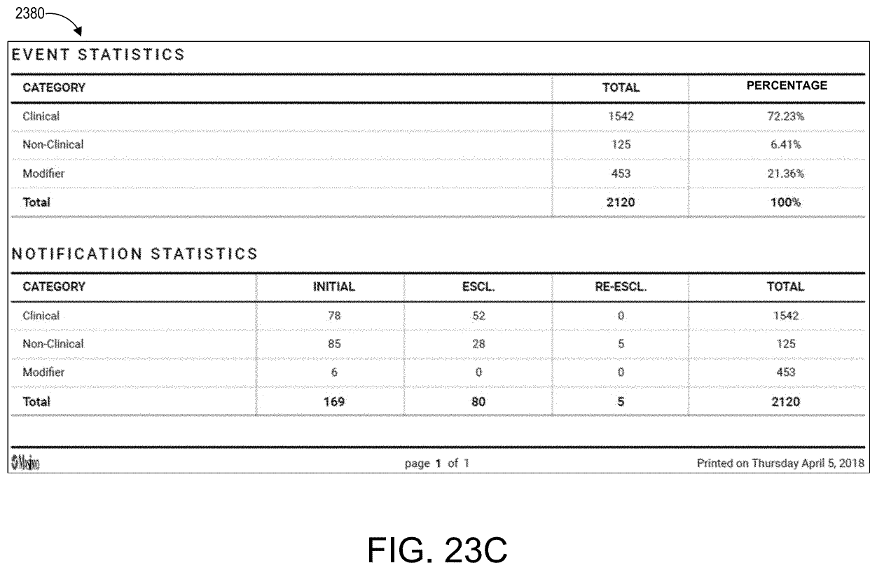

FIG. 22 depicts an example export user interface 2200 that may be presented by the clinician devices 104 described above. The export user interface 2200 can include export user interface elements 2202, which can include the export options 2204A, 2204B, 2204C. Example exporting can include printing one or more user interface elements to a document. The export user interface 2200 can be similar to the analytics user interface 13A of FIG. 13 described above. In particular, a clinician can use the export options 2204A, 2204B, 2204C to selectively export portions of any of the analytics user interfaces described above, such as portions of the analytics user interface 13A of FIG. 13. As shown, a clinician can select one or more of the export options 2204A, 2204B, 2204C to export user interface elements, such as event visualizations, notification visualizations, event analytics data, notification analytics data, patient metadata, or patient device metadata. Any of the user interfaces described above with respect to FIGS. 2 through 7A-7C, 9A-9B, 10, 11A-11C, and 13A-13B through 21A-21D can be exported via the export user interface elements 2202. However, depending on the user interface being exported, the export options 2204A, 2204B, 2204C may dynamically update to correspond to the particular user interface elements of the selected user interface. Example exported documents are described in further detail below with respect to FIGS. 23A-23C through 27A-27B.

### IX. Additional Example Analytics Documents

FIGS. 23A-23C, 24A-24C, 25A-25D, 26A-26C, and 27A-27B depict example analytics portions of an analytics document. The analytics documents can be in a data format, such as a Portable Document Format (PDF), Tagged Image File Format (TFF), a delimited file, a spreadsheet format, or a word processing document format. Turning to FIGS. 23A-23C, the analytics portions 2300, 2340, 2380 are depicted. One or more of the analytics portions 2300, 2340, 2380 of FIGS. 23A, 23B, 23C, respectively, can be generated by the analytics system 160. The analytics portions 2300, 2340, 2380 of FIGS. 23A, 23B, 23C, respectively, can constitute an analytics document together or singly. In some configurations, the analytics portion 2300 of FIG. 23A can be shown above the analytics portion 2340 of FIG. 23B, or the analytics portion 2340 of FIG. 23B can be shown above the analytics portion 2380 of FIG. 23C.

The analytics portions 2300, 2340, 2380 of FIGS. 23A-23C can include dashboard analytics for a clinical setting. In FIG. 23A, the analytics portion 2300 can include a header identifying input parameters associated with the analytics, such as a selected group or time range. Turning to FIG. 23B, the dashboard analytics portion 2340 of FIG. 23B can correspond to, include similar elements from, or be generated from the dashboard analytics user interface 1300 of FIG. 13A. The dashboard analytics portion 2340 can include visual event summary indicators or historical metadata indicators.

Turning to FIG. 23C, the dashboard analytics portion 2380 of FIG. 23C can include event statistics for different event categories. As shown, statistics for a first event category can include clinical, non-clinical, or modifier event statistics. In particular, the dashboard analytics portion 2380 can correspond to a portion of a dashboard user interface that includes indicators for a first quantity of historical events corresponding to a first event type (such as 1543 clinical events) and a second quantity of historical events corresponding to a second event type (such as 125 non-clinical events or 453 modifier events). As shown, statistics for a second event category can include clinical, non-clinical, or modifier event statistics that can be categorized by notification type, such as initial, escalation, and re-escalation. In particular, the dashboard analytics portion 2380 can correspond to a portion of a dashboard user interface that includes indicators for a first quantity of notifications corresponding to a first notification category (such as 1542 clinical notifications) and a second quantity of notifications corresponding to a second notification category (such as 125 non-clinical notifications or 453 modifier notifications).

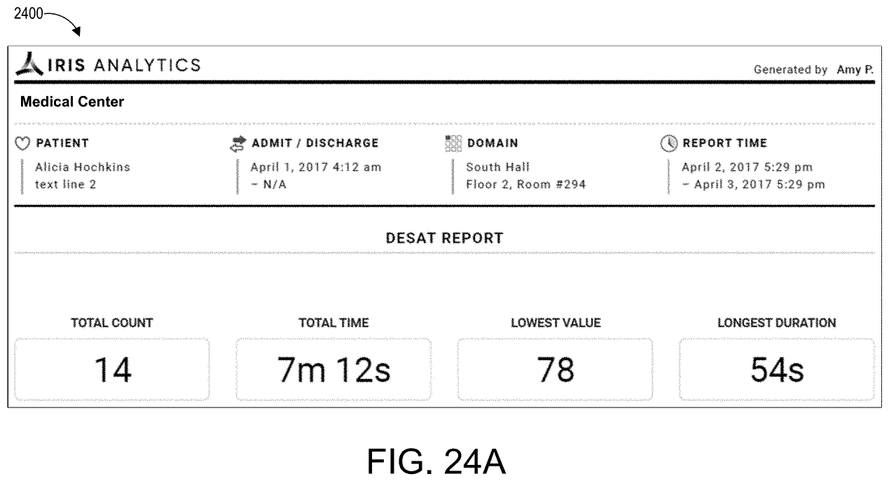

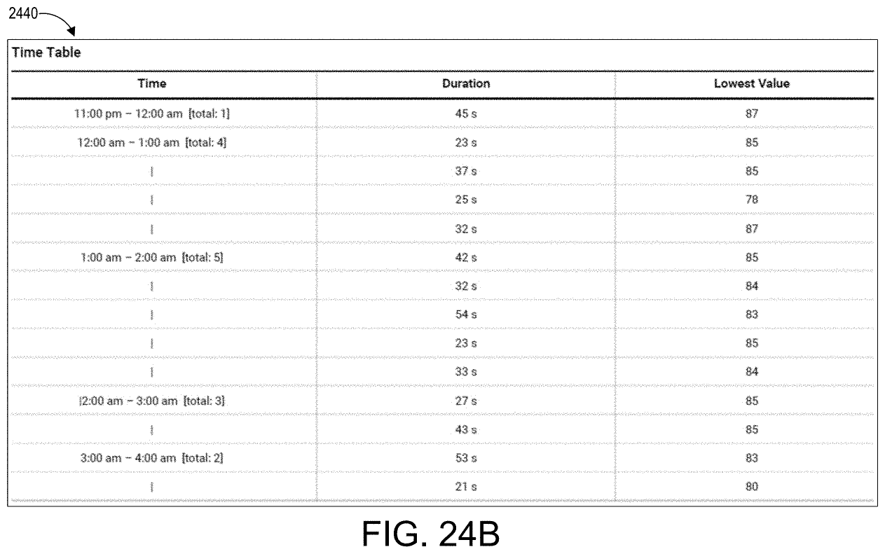

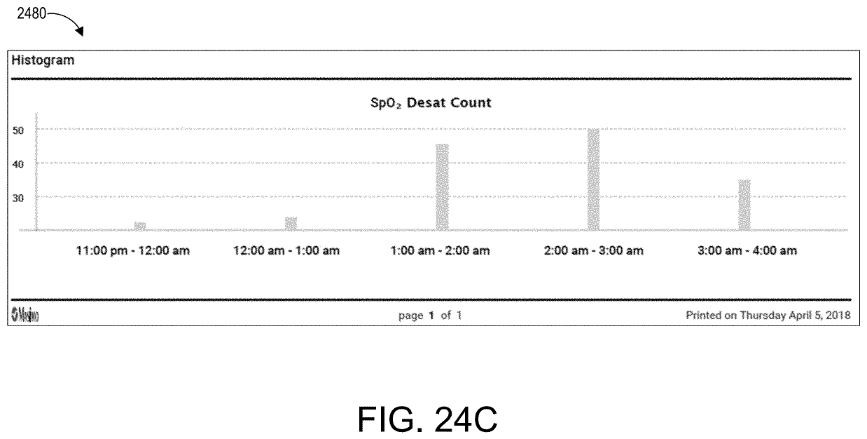

Turning to FIGS. 24A-24C, the analytics portions 2400, 2440, 2480 are depicted. One or more of the analytics portions 2400, 2440, 2480 of FIGS. 24A, 24B, and 24C, respectively, can be generated by the analytics system 160. The analytics portions 2400, 2440, 2480 of FIGS. 24A, 24B, and 24C, respectively, can constitute an analytics document together or singly. In some configurations, the analytics portion 2400 of FIG. 24A can be shown above the analytics portion 2440 of FIG. 24B, or the analytics portion 2440 of FIG. 24B can be shown above the analytics portion 2480 of FIG. 24C.

The analytics portions 2400, 2440, 2480 of FIGS. 24A-24C can include desaturation analytics. The analytics portions 2400, 2440, 2480 of FIGS. 24A, 24B, and 24C can correspond to, include similar elements from, or be generated from the desaturation user interfaces 700, 710, 750 of FIGS. 7A-7C. In FIG. 24A, the desaturation analytics portion 2400 can include a header identifying input parameters associated with the desaturation analytics, such as a selected patient, group, or time range. The desaturation analytics portion 2400 can also include analytics such as, a count of desaturation events, a cumulative time of desaturation events, a highest or lowest value of a desaturation event, or a shortest or longest time for a desaturation event. Turning to FIG. 24B, the desaturation analytics portion 2440 can include a time table of desaturation events. Turning to FIG. 24C, the desaturation analytics portion 2480 of FIG. 24C can include a histogram of desaturation event counts grouped by a time unit.

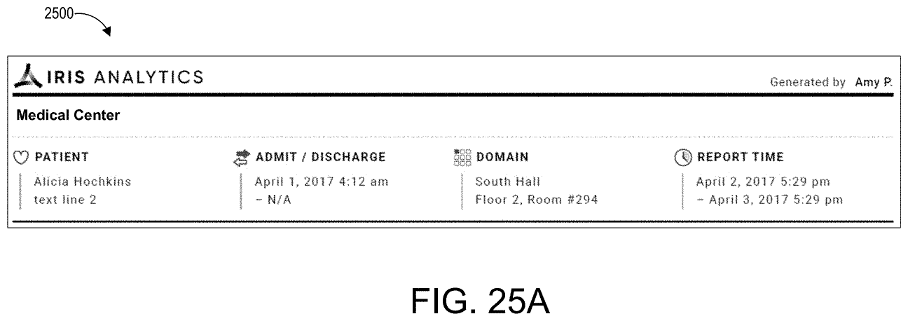

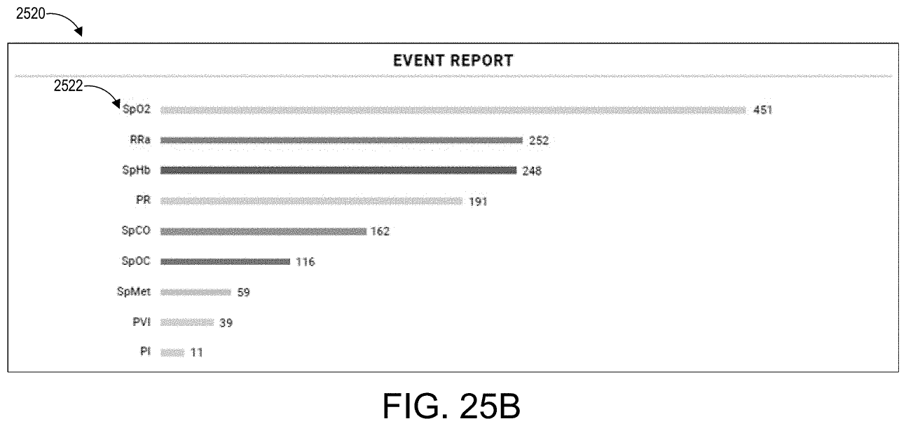

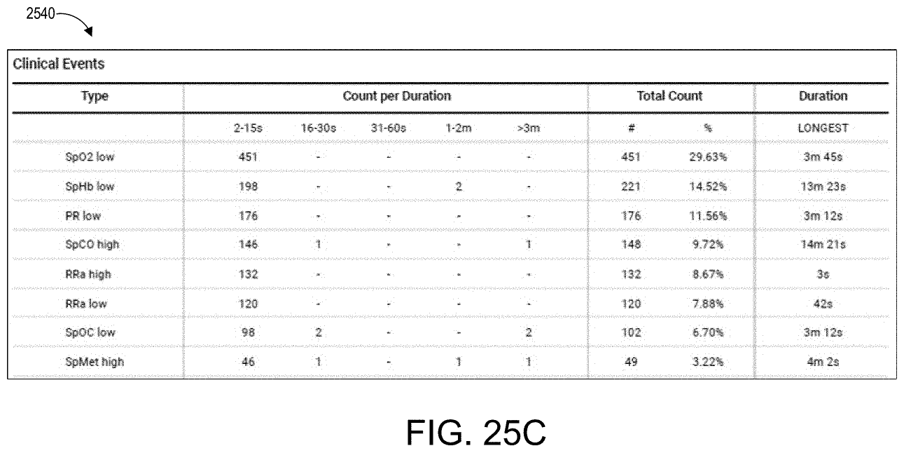

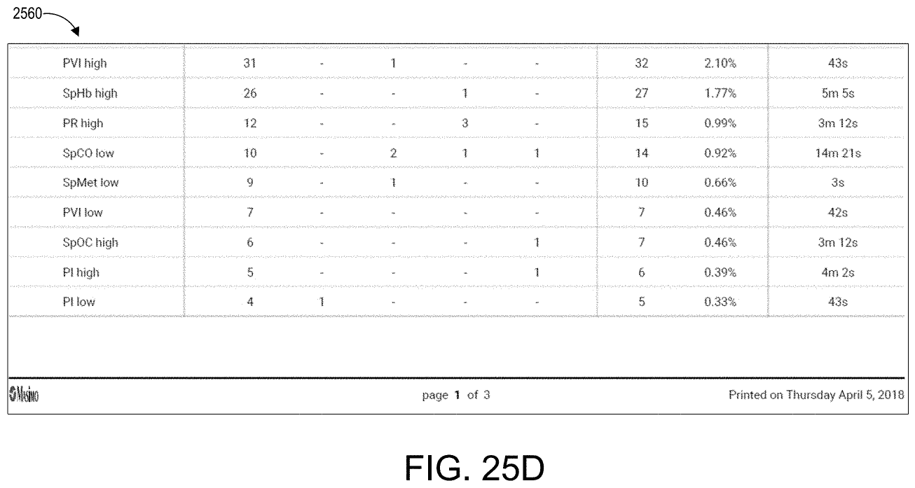

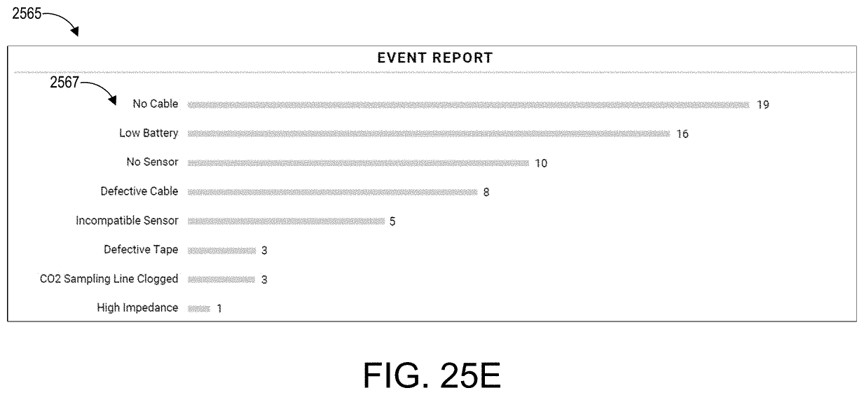

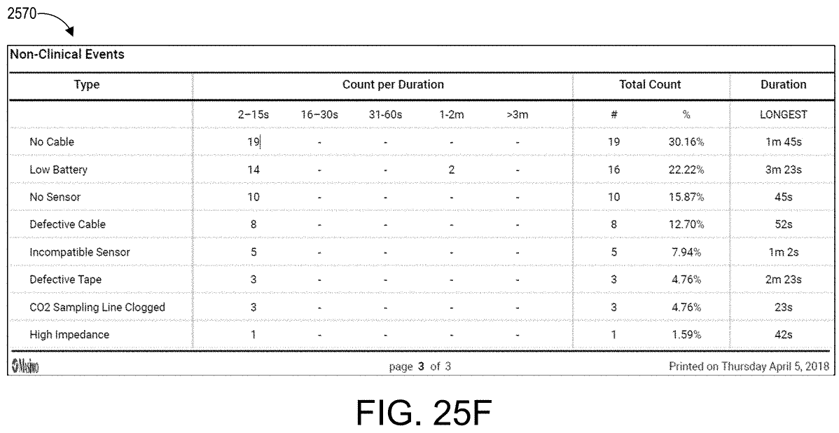

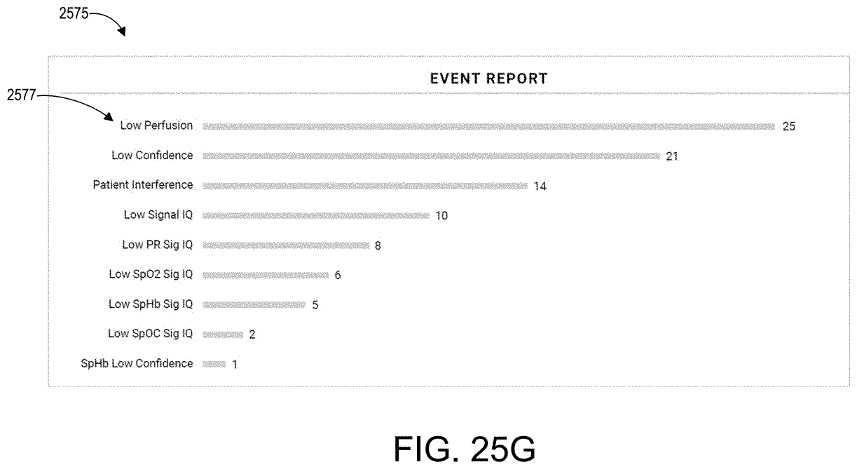

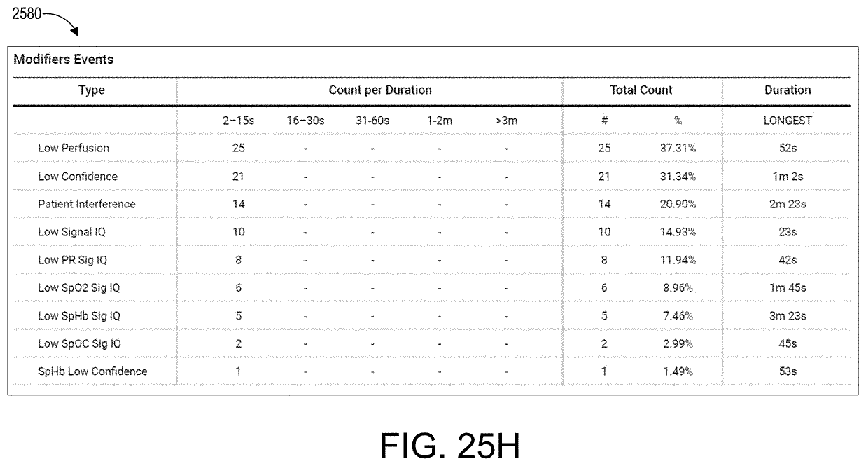

Turning to FIGS. 25A-25H, the analytics portions 2500, 2520, 2540, 2560, 2565, 2570, 2575, 2580 are depicted. One or more of the analytics portions 2500, 2520, 2540, 2560, 2565, 2570, 2575, 2580 of FIGS. 25A, 25B, 25C, 25D, 25E, 25F, 25G, and 25H respectively, can be generated by the analytics system 160. The analytics portions 2500, 2520, 2540, 2560, 2565, 2570, 2575, 2580 of FIGS. 25A, 25B, 25C, 25D, 25E, 25F, 25G, and 25H respectively, can constitute an analytics document together or singly. In some configurations, the analytics portion 2500 of FIG. 25A can be shown above the analytics portion 2520 of FIG. 25B, the analytics portion 2520 of FIG. 25B can be shown above the analytics portion 2540 of FIG. 25C, the analytics portion 2540 of FIG. 25C can be shown above the analytics portion 2560 of FIG. 25D, the analytics portion 2565 of FIG. 25E can be shown above the analytics portion 2570 of FIG. 25F, or the analytics portion 2575 of FIG. 25G can be shown above the analytics portion 2580 of FIG. 25H.

The analytics portions 2500, 2520, 2540, 2560, 2565, 2570, 2575, 2580 of FIGS. 25A-25D can include event analytics. In FIG. 25A, the desaturation analytics portion 2500 can include a header identifying input parameters associated with the event analytics, such as a selected patient, group, or time range. Turning to FIG. 25B, the analytics portion 2520 can include a visual event summary indicator 2522. The visual event summary indicator 2522 can include visual representations corresponding to respective quantities of clinical events for physiological parameters, such as a count of oxygen saturation (SpO2) events (here 451 events), a count of acoustic respiration rate (RRa®) events (here 252 events), a count of total hemoglobin (SpHb®) events (here 248 events), a count of pulse rate (PR) events (here 191 events), a count of carboxyhemoglobin (SpCO) events (here 162 events), a count of oxygen content (SpOC™), a count of methemoglobin (SpMet®) events (here 59 events), a count of pulse variability index (PVi®) events (here 39 events), or a count of perfusion index (Pi) events (here 11 events).

Turning to FIG. 25C, the analytics portion 2540 can include a statistical summary of clinical events. Turning to FIG. 25D, the analytics portion 2560 can include further statistical summary entries for clinical events, which can be a continuation of the statistical summary from the analytics portion 2540 of FIG. 25C. The analytics portions 2540, 2560 of FIGS. 25C, and 25D can correspond to, include similar elements from, or be generated from the analytics user interface 1420 of FIG. 14B. Turning to FIG. 25E, the analytics portion 2565 can include a visual event summary indicator 2567. The visual event summary indicator 2567 can include visual representations corresponding to respective quantities of non-clinical events. Turning to FIG. 25F, the analytics portion 2570 can include a statistical summary of non-clinical events. The analytics portion 2570 of FIG. 25F can correspond to, include similar elements from, or be generated from the analytics user interface 1440 of FIG. 14C. Turning to FIG. 25G, the analytics portion 2575 can include a visual event summary indicator 2577. The visual event summary indicator 2577 can include visual representations corresponding to respective quantities of modifier events. Turning to FIG. 25H, the analytics portion 2580 can include a statistical summary of modifier events. The analytics portion 2580 of FIG. 25H can correspond to, include similar elements from, or be generated from the analytics user interface 1460 of FIG. 14D.

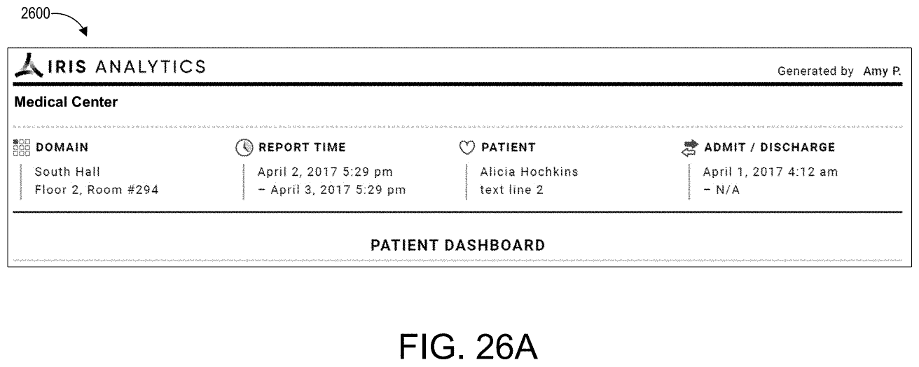

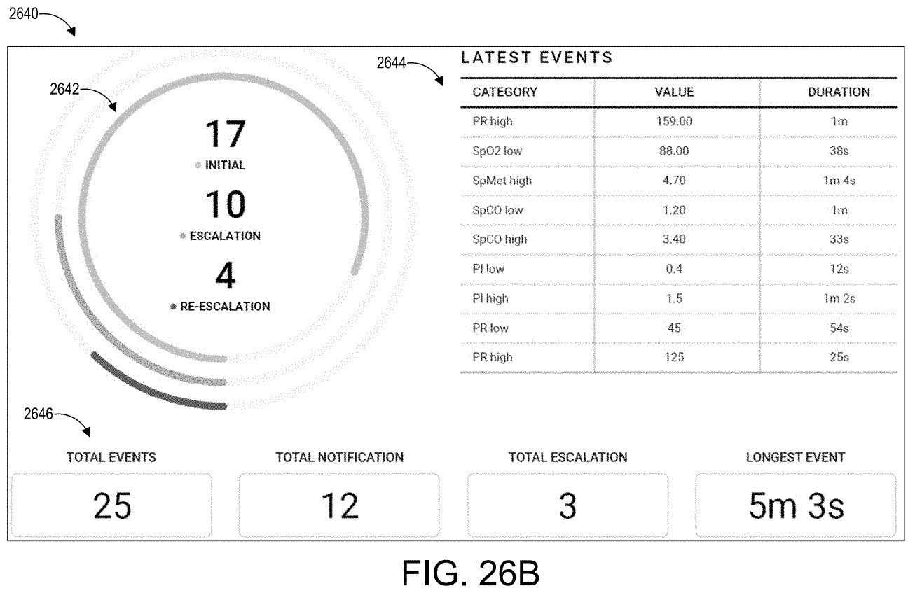

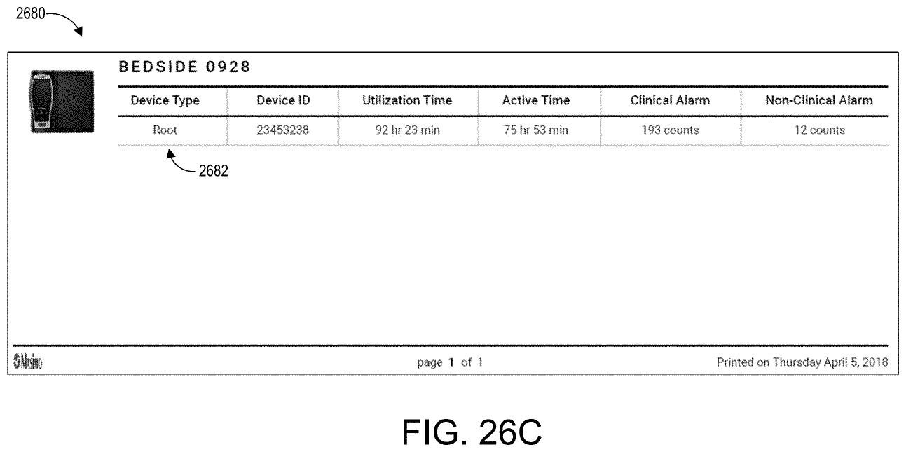

Turning to FIGS. 26A-26C, the analytics portions 2600, 2640, 2680 are depicted. The analytics portions 2600, 2640, 2680 can correspond to a user interface, such as a patient dashboard user interface. One or more of the analytics portions 2600, 2640, 2680 of FIGS. 26A, 26B, and 26C, respectively, can be generated by the analytics system 160. The analytics portions 2600, 2640, 2680 of FIGS. 26A, 26B, and 26C, respectively, can constitute an analytics document together or singly. In some configurations, the analytics portion 2600 of FIG. 26A can be shown above the analytics portion 2640 of FIG. 26B, or the analytics portion 2640 of FIG. 26B can be shown above the analytics portion 2680 of FIG. 26C.

The analytics portions 2600, 2640, 2680 of FIGS. 26A-26C can include patient analytics. In FIG. 26A, the analytics portion 2600 can include a header identifying input parameters associated with patient analytics, such as a selected patient, group, or time range. Turning to FIG. 26B, the patient analytics portion 2640 can include a visual event summary indicator 2642, an events table 2644, or historical metadata indicators 2646. Turning to FIG. 26C, the patient analytics portion 2680 of FIG. 26C can include a patient device entry 2682. The patient device entry 2682 can include information regarding a patient device type, a device identifier, utilization time of the patient device, active time of the patient device, a number of clinical alarms from the patient device, or a number of non-clinical alarms from the patient device.

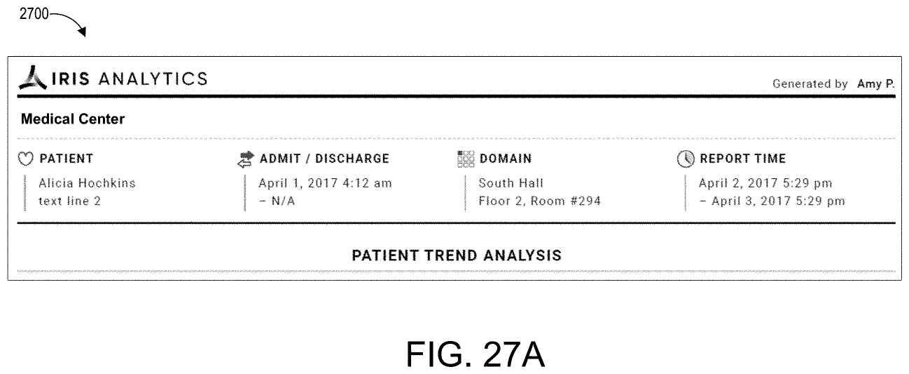

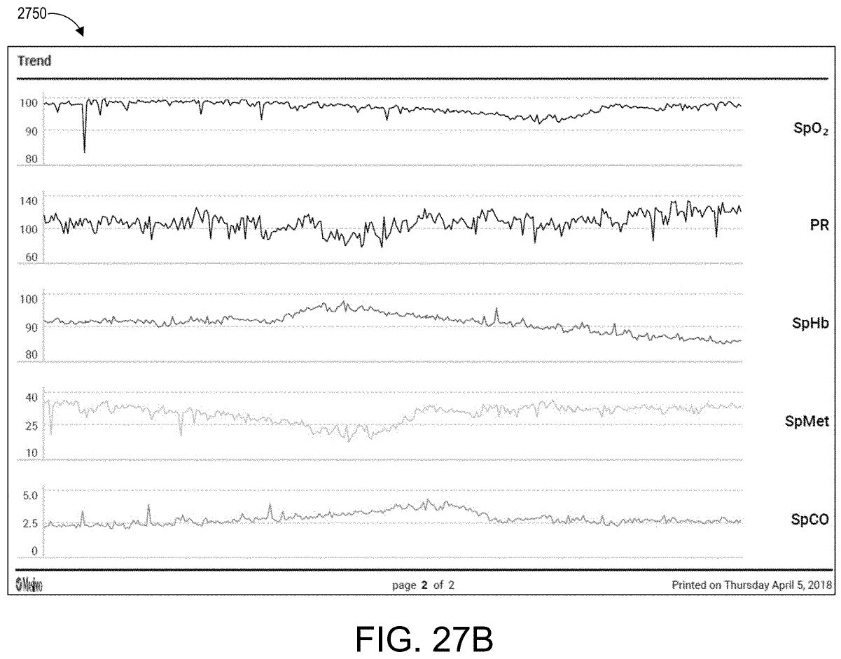

Turning to FIGS. 27A-27B, the analytics portions 2700, 2750 are depicted. One or more of the analytics portions 2700, 2750 of FIGS. 27A and 27B, respectively, can be generated by the analytics system 160. The analytics portions 2700, 2750 of FIGS. 27A and 27B, respectively, can constitute an analytics document together or singly. In some configurations, the analytics portion 2700 of FIG. 27A can be shown above the analytics portion 2750 of FIG. 27B. The analytics portion 2750 of FIG. 27B can correspond to, include similar elements from, or be generated from the analytics user interface 1180 of FIG. 11C. The analytics portions 2700, 2750 of FIGS. 27A-27B can include patient trend analytics. In FIG. 27A, the analytics portion 2700 can include a header identifying input parameters associated with patient trend analytics, such as a selected patient, group, or time range. Turning to FIG. 27B, the patient trend analytics portion 2750 can include a visualization of one or more physiological parameter values over time.

### X. Example Analytics Process

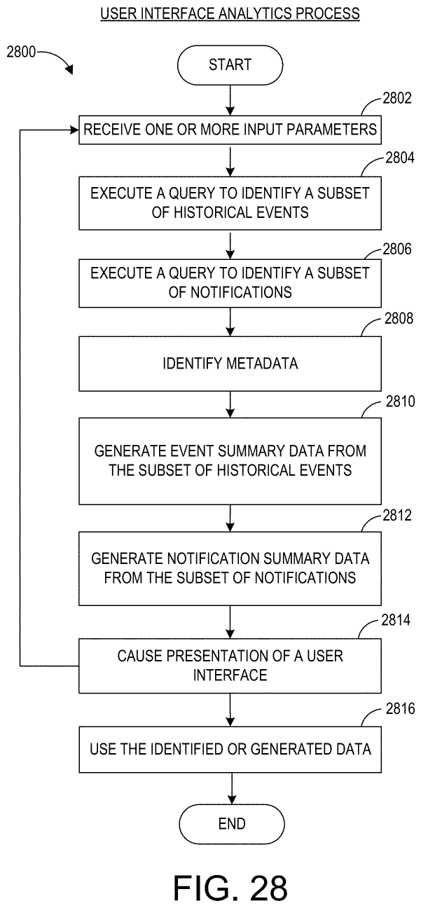

Turning to FIG. 28, an example user interface analytics process 2800 is shown. The user interface analytics process 2800 may be implemented by the analytics system 160. For convenience, the user interface analytics process 2800 will be described in the context of the analytics system 160, although other computing systems not described herein may implement the user interface analytics process 2800. The user interface analytics process 2800 can advantageously provide improved patient care by enabling identification or enabling more rapid identification of patient issues or enabling further actions, such as the implementation of corrective measures, as described herein.

At block 2802, one or more input parameters can be received. In particular, the analytics system 160 can receive one or more input parameters, such as user input. Block 2802 of FIG. 28 can be similar to blocks 802, 804, 806 of FIG. 8 or blocks 1202, 1204, 1206 of FIG. 12. A clinician can select a patient, a group, a patient device, or a time range to generate analytics for one or more patients and a user interface can receive the selected input parameter. One or more input parameters can include desaturation parameters, trend analysis parameters, or other input parameters related to physiological measurements. As described above with respect to the user interface 600 of FIG. 6, respectively, a user interface can receive user input, and the user input can include a selected patient. As described above with respect to the user interface 200 of FIG. 2, a user interface can receive user input, and the user input can include a group. As described above with respect to the user interfaces 300, 1100 of FIGS. 3 and 11A, respectively, a user interface can receive user input and the user input can include a time range. A selected patient or a group identifying a subset of patient devices or patients can be used to identify a subset of historical events that can be used for analytics purposes. A time range can be used to identify a subset of historical events that can be used for trend analytics, such as the historical events that occurred or that includes a timestamp within the start time and end time of the time range. A time range can include a start time and an end time. Identifying a subset of analytics may be advantageous because determining or presenting analytics for a larger set of patient devices, patients, or historical events may be computationally more expensive or impractical, may result in a more cluttered user interface, or may make it slower for a clinician to access desired data. Identifying a subset of analytics may therefore be computationally faster than identifying analytics for a larger set of patient devices, patients, or historical events.

At block 2804, a query can be executed to identify a subset of historical events. In particular, the analytics system 160 can execute a query to identify a subset of historical events. Block 2804 of FIG. 28 can be similar to blocks 808, 810 of FIG. 8 or blocks 1208, 1210 of FIG. 12. The query can include input parameters, such as a patient identifier, a group, a patient device, a start time, or an end time. Executing the query can include identifying, from the historical events in the data storage 120, a subset of historical events from the patient, group, or patient device and within the start time and the end time. The query can include input parameters, such as a patient identifier (for example, “2145293587”), a group, or a patient device identifier, and a start time (such as Aug. 14, 2018 at 5:36 PM) and an end time (such as Aug. 16, 2018 at 2:12 AM). Executing the query can result in identifying historical events, such as clinical events, non-clinical events, or modifier events. The query can be in a format configured for a database, such as in a SQL format or an object-oriented database query format.

At block 2806, a query can be executed to identify a subset of notifications, a particular type of historical event. In particular, the analytics system 160 can execute a query to identify a subset of notifications. The query can include input parameters, such as a patient identifier, a group, a patient device, a start time, or an end time. Executing the query can include identifying, from notifications in the data storage 120, a subset of notifications from the patient, group, or patient device and within the start time and the end time. The query can include input parameters, such as a patient identifier (for example, “2145293587”), a group, or a patient device identifier, and a start time and an end time. Executing the query can result in identifying notifications, such as initial notifications, escalation notifications, or re-escalation notifications. The query can be in a format configured for a database, such as in a SQL format or an object-oriented database query format.

At block 2808, metadata can be identified. In particular, the analytics system 160 can identify metadata. Example metadata can include patient, patient device, or clinician metadata. The analytics system can identify metadata based on determined historical events. The analytics system can identify patient, patient device, or clinician metadata associated with historical events, such as information related to a patient device that generated an event for a patient or information related to a clinician that received or responded to an event.

At block 2810, event summary data can be generated from the subset of historical events. In particular, the analytics system 160 can generate event summary data from the subset of historical events. Block 2810 of FIG. 28 can be similar to block 812 of FIG. 8 or block 1212 of FIG. 12. The analytics system 160 can calculate a quantity of historical events from the subset of historical events. The analytics system 160 can quantify the identified historical events, such as by calculating statistical summary data that can include a total number of historical events, a total time of the historical events, a longest in time historical event, a highest value historical event, a lowest value historical event, or an average number of historical events per time unit. The analytics system 160 can generate other statistical measures of historical events, such as a median, mode, weighted average, weighted median, weighted mode, or standard deviation. The generated event summary data can provide a clinician greater insight into clinical issues and thereby can improve patient care.

In particular, the analytics system 160 can calculate a quantity of historical events from the subset of historical events based on an event category, such as a first event category or a second event category that is different from the first event category. The analytics system 160 can calculate multiple quantities of historical events (such as a first quantity, a second quantity, a third quantity, etc.) based on a respective event category. Example event categories can include a clinical event category, a non-clinical event category, or a modifier event category.

In particular, the analytics system 160 can calculate a quantity of historical events from the subset of historical events for particular patients, such as a first patient or a second patient that is different from the first patient. The analytics system 160 can calculate multiple quantities of historical events for each patient, such as a first quantity for a first patient, a second quantity for a second patient, a third quantity for a third patient, etc. The analytics system 160 can further select summary data for presentation based on the calculated quantities, such as a presentation of the “10 Highest Patient Event Count.” The analytics system 160 can determine that a first quantity for the first patient is greater than a second quantity for a second patient. Accordingly, the analytics system 160 can select the first quantity instead of the second quantity, which can indicate that the first patient as a greater number of events than the second patient.

In particular, the analytics system 160 can calculate a quantity of historical events from the subset of historical events for a particular patient device 104, such as a first patient device for a first patient. The analytics system 160 can calculate a quantity of historical events that originated from or are associated with a first patient device, such as clinical, non-clinical, or modifier alarms that originated from the patient device 104. The calculated summary data for the patient device 104 can be used in a user interface, such as a patient dashboard user interface for a first patient that is associated with the first patient device.

At block 2812, notification summary data can be generated from the subset of notifications. In particular, the analytics system 160 can generate notification summary data from the subset of notifications. The analytics system 160 can calculate a quantity of notifications from the subset of notifications. The analytics system 160 can quantify the identified notifications, such as by calculating statistical summary data that can include a total number of notifications, a total duration of notifications, or an average number of notifications per time unit. The analytics system 160 can generate other statistical measures of historical events, such as a median, mode, weighted average, weighted median, weighted mode, or standard deviation. The generated notification summary data can provide a clinician greater insight into clinical issues and thereby can improve patient care.

In particular, the analytics system 160 can calculate a quantity of notifications from the subset of notifications based on a notification category, such as a first notification category or a second notification category that is different from the first notification category. Example notification categories can include a clinical notification category, a non-clinical notification category, or a modifier notification category. The analytics system 160 can calculate a quantity of notifications from the subset of notifications based on a notification type, such as a first notification type or a second notification type that is different from the first notification type. Example notification types can include an initial notification type, an escalation notification type, or a re-escalation notification type. A notification can correspond to one or more of a notification category or a notification type. The analytics system 160 can calculate multiple quantities of notifications (such as a first quantity, a second quantity, a third quantity, etc.) based on a respective notification category or notification type.

At block 2814, a user interface can be presented. In particular, the analytics system 160 can cause presentation of a user interface, such as a dashboard user interfaces or any of the user interfaces described above. Example user interfaces include any of the user interfaces of FIGS. 2 through 7A-7C, 9A-9B, 10, 11A-11C, and 13A-13B through 22. Block 2814 of FIG. 28 can be similar to block 814 of FIG. 8 or block 1214 of FIG. 12. The analytics system 160 can cause presentation of the user interface on the clinician device 104. The user interface can include any of the data described above with respect to the previous blocks 2802, 2804, 2806, 2808, such as the event summary data, the notification summary data, the identified historical events, the identified notifications, or metadata. In particular, a user interface, such as a dashboard user interface can include an indicator for a quantity of historical events (such as multiple indicators for respective quantities of historical events grouped by event type) or a quantity of notifications (such as multiple indicators for respective quantities of notifications grouped by notification type). The presented data can enable a clinician to address any of the identified clinical issues. A clinician can export one or more of the user interfaces to an analytics document, such as any of the example analytics documents described above with respect to FIGS. 23A-23C, 24A-24C, 25A-25D, 26A-26C, and 27A-27B.

As shown in the process 2800, additional user input can be received at block 2802 and additional blocks 2804, 2806, 2808, 2810, 2812, 2814 can be performed. Additional user input can include updated input parameters, such as, an updated time range, or an updated patient or group. Accordingly, updated historical events can be determined based on the updated input parameters or updated event summary data or updated notification summary data can be determined based on the updated input parameters, such as an updated time range. In particular, a clinician can narrow down a time range or can filter from a group to a particular patient based on the initial analytics data to review more narrowly refined analytics data. The analytics system 10 can present updated user interfaces, such as an updated dashboard user interface or any of the user interfaces described above.

At block 2816, identified or generated data can be used. In particular, the analytics system 160 can use the identified or generated data from the previous blocks 2804, 2806, 2808, 2810, 2812. Using the identified or generated data can include generating or exporting a report including the data. A clinician can also use the identified or generated data from the previous blocks 2804, 2806, 2808, 2810, 2812. Block 2816 of FIG. 28 can be similar to block 816 of FIG. 8 or block 1216 of FIG. 12. The analytics system 160 can use the identified or generated data to adjust or set an alarm limit for a specific patient. For example, if the patient's device is alarming too often, which can be indicated by the generated summary data, then the analytics system 160 can adjust an alarm limit to reduce the number of alarms. Likewise, a clinician viewing the summary data, can identify that a patient's device is alarming too often and can adjust an alarm limit accordingly. The analytics system 160 or a clinician can compare events with generated distributions of events, such as histograms, to determine whether an alarm is a true alarm or not. For particular physiological parameters, the analytics system 160 can include logic to determine that where particular events occur at a rate above a particular threshold then the conditions for those events should not trigger further alarms. Conversely, a clinician can compare events with generated distributions of events and determine that there are not enough alarms. A clinician can view the distributions of historical events and determine that the alarm limits for a physiological parameter should be adjusted to generate additional alarms for the historical events.

### XI. Example Analytics System

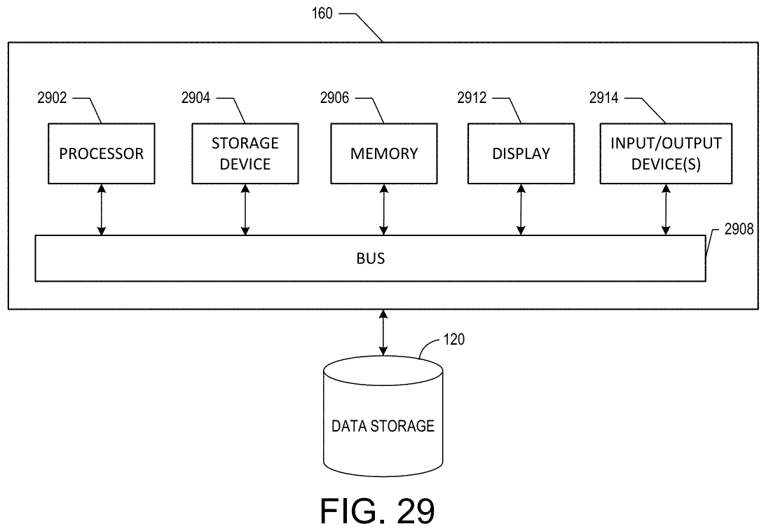

FIG. 29 is a block diagram that illustrates example components of the analytics system 160. While the analytics system 160 of FIG. 29 is depicted as a single device, the analytics system 160 may be implemented in a server cluster, server farm, data center, mainframe, cloud computing environment, or the like. The analytics system 160 can include any number of devices that operate as distributed computing resources that provides services, such as storage, computing, networking, and so on.

The analytics system 160 can include a hardware processor 2902, a data storage device 2904, a memory device 2906, a bus 2908, a display 2912, and one or more input/output devices 2914. A processor 2902 can also be implemented as a combination of computing devices, e.g., a combination of a digital signal processor and a microprocessor, a plurality of microprocessors, one or more microprocessors in conjunction with a digital signal processor, or any other such configuration. The processor 2902 can be configured, among other things, to process data, execute instructions to perform one or more functions, such as process one or more physiological signals to obtain one or measurements, as described herein. The data storage device 2904 can include a magnetic disk, optical disk, or flash drive, etc., and may be provided and coupled to the bus 2908 for storing information and instructions. The memory 2906 can include one or more memory devices that store data, including without limitation, random access memory (RAM) and read-only memory (ROM). The analytics system 160 may be coupled via the bus 2908 to a display 2912, such as a LCD display or touch screen, for displaying information to a user, such as a clinician. The analytics system 160 may be coupled via the bus 2908 to one or more input/output devices 2914. The input device 2914 can include, but is not limited to, a keyboard, mouse, digital pen, microphone, touch screen, gesture recognition system, voice recognition system, imaging device (which may capture eye, hand, head, or body tracking data and/or placement), gamepad, accelerometer, or gyroscope.

In some embodiments, the analytics system 160 can communicate with the data storage 120. When the analytics system 160 can communicate with the data storage 120, one or more historical events can be transmitted to the analytics system 160. Physiological signals can be processed into representations of physiological parameters and/or measurements and stored in the data storage 120. The signals can be processed into multiple readings of each physiological parameter over a period of time such as, for example, 10 minutes, 30 minutes, or 1 hour. Additional details regarding processing of physiological signals to obtain measurements are described in at least U.S. patent application Ser. No. 11/366,208, filed Mar. 1, 2006, titled Noninvasive Multi-Parameter Patient Monitor, now issued as U.S. Pat. No. 8,130,105, and U.S. patent application Ser. No. 12/559,815, filed Sep. 15, 2009, titled Patient Monitor Including Multi-Parameter Graphical Display, now issued as U.S. Pat. No. 8,911,377, which is hereby incorporated by reference in its entirety.

A temperature sensor may capture one or more physiological signals related to a patient's temperature, such as a body core temperature. The one or more physiological signals can be processed to measure the patient's body core temperature, which is a vital sign used by clinicians to monitor and manage patients' conditions. The temperature sensor can include a thermocouple, a temperature-measuring device having two dissimilar conductors or semiconductors that contact each other at one or more spots. A temperature differential can be experienced by the different conductors. The thermocouple can produce a voltage when the contact spot differs from a reference temperature. Thermocouples may be self-powered and therefore may not require an external power source for operation. In some embodiments, the temperature sensor can include a thermistor. A thermistor is a type of resistor whose resistance value can vary depending on its temperature. Thermistors typically offer a high degree of precision within a limited temperature range.

An acoustic respiration sensor may capture one or more physiological signals related to vibrational motion from the patient's body (e.g., the patient's chest) that are indicative of various physiologic parameters and/or conditions, including without limitation, heart rate, respiration rate, snoring, coughing, choking, wheezing, and respiratory obstruction (e.g., apneic events). Additional details regarding an example acoustic respiration sensor are described in U.S. patent application Ser. No. 12/643,939, filed Dec. 21, 2009, titled Acoustic Sensor Assembly, now issued as U.S. Pat. No. 8,771,204, attorney docket MCAN.030A, which is hereby incorporated by reference in its entirety.

An electrocardiogram (ECG) sensor may capture one or more physiological signals related to cardiac activity. One or more physiological signals can be processed to measure the patient's cardiac activity. In some embodiments, the ECG signal can be processed to detect arrhythmias, such as bradycardia, tachyarrhythmia, or ventricular fibrillation.

An oximetry sensor may capture one or more physiological signals related to pulse oximetry. The one or more physiological signals can be processed to measure the patient's pulse oximetry, a widely accepted noninvasive procedure for measuring the oxygen saturation level of arterial blood, an indicator of a person's oxygen supply. Example oximetry sensor(s) 1324 include an optical sensor clipped onto a portion of the patient's body (such as, for example, a fingertip, an ear lobe, and/or a nostril). The signals can be processed to measure the relative volume of oxygenated hemoglobin in pulsatile arterial blood flowing within the portion of the body being sensed, which includes measurements such as Oxygen saturation (SpO2), pulse rate, a plethysmograph waveform, perfusion index (PI), pleth variability index (PVi®), methemoglobin (MetHb), carboxyhemoglobin (CoHb), total hemoglobin (tHb), and/or glucose.

### XII. Additional Embodiments and Terminology

While the present disclosure at times discusses analytics and user interfaces in the context of a particular physiological parameter or a particular context, such as desaturation, the apparatuses, systems, and methods described herein may be agnostic to the particular context, and, therefore, may be used in a different context or for a different physiological parameter, such as a respiration rate.

Conditional language used herein, such as, among others, “can,” “might,” “may,” “e.g.,” “for example,” and the like, unless specifically stated otherwise, or otherwise understood within the context as used, is generally intended to convey that certain embodiments include, while other embodiments do not include, certain features, elements, or states. Thus, such conditional language is not generally intended to imply that features, elements or states are in any way required for one or more embodiments.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, and/or Z). Such disjunctive language is not generally intended to, and should not, imply that certain embodiments require at least one of X, at least one of Y, or at least one of Z to each be present. Thus, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

The term “a” as used herein should be given an inclusive rather than exclusive interpretation. For example, unless specifically noted, the term “a” should not be understood to mean “exactly one” or “one and only one”; instead, the term “a” means “one or more” or “at least one,” whether used in the claims or elsewhere in the specification and regardless of uses of quantifiers such as “at least one,” “one or more,” or “a plurality” elsewhere in the claims or specification.

The terms “comprising,” “including,” “having,” and the like are synonymous and are used inclusively, in an open-ended fashion, and do not exclude additional elements, features, acts, operations, and so forth.

While the above detailed description has shown, described, and pointed out novel features as applied to various embodiments, it will be understood that various omissions, substitutions, and changes in the form and details of the devices or algorithms illustrated can be made without departing from the spirit of the disclosure. As will be recognized, certain embodiments described herein can be embodied within a form that does not provide all of the features and benefits set forth herein, as some features can be used or practiced separately from others.

What is claimed is:

1. A system for investigating patient desaturations, the system comprising:
data storage that stores a plurality of historical events, wherein each historical event from the plurality of historical events comprises an oxygen saturation parameter value for a patient at a timestamp; and
a hardware processor configured to:
receive, from a user interface, first user input comprising a first domain, the first domain indicating a group of patient devices;
receive, from the user interface, second user input comprising a time range, the time range comprising a start time and an end time;
receive, from the user interface, third user input comprising (i) an oxygen drop percentage, (ii) a time window, and (iii) a duration;
execute a query comprising input parameters, the input parameters comprising the first domain, the start time, and the end time, wherein executing the query further comprises:
identifying, from the plurality of historical events, a first subset of the historical events from the first domain and within the start time and the end time;
identify, from the first subset of the historical events, a second subset of the historical events, wherein identifying the second subset of the historical events further comprises:
identifying, for a particular patient, respective oxygen saturation physiological parameter values and timestamps within the oxygen drop percentage, the time window, and the duration;
generate desaturation summary data comprising a quantity of desaturation events for each patient from the second subset of the historical events; and
output the desaturation summary data for presentation in a desaturation summary user interface.

2. The system of claim 1, wherein the desaturation summary user interface comprises:
for each patient from the second subset of the historical events,
a patient indicator,
a domain indicator, and
a count indicator for the quantity of desaturation events; and
wherein the hardware processor is further configured to:
receive, from the desaturation summary user interface, fourth user input comprising a selection for a first patient; and
cause presentation of some data from the second subset of the historical events for the first patient in a desaturation detail user interface.

3. The system of claim 2, wherein the desaturation detail user interface further comprises:
a visual entry for a first desaturation event for the first patient, the visual entry comprising: (i) a first indicator representing a first duration of the first desaturation event, and (ii) a second indicator representing a first oxygen saturation value for the first desaturation event.

4. The system of claim 2, wherein the desaturation detail user interface further comprises:
a visual representation of a distribution for a plurality of desaturation events for the first patient grouped by a time unit.

5. A method for generating patient analytics, the method comprising:
under control of a hardware processor,
receiving, from a user interface, first user input comprising a first domain, the first domain indicating a group of patient devices;
receiving, from the user interface, second user input comprising a time range, the time range comprising a start time and an end time;
executing (i) a first query comprising input parameters and (ii) and a second query comprising the input parameters, the input parameters comprising the first domain, the start time, and the end time,
wherein executing the first query further comprises:
identifying, from a plurality of historical events, a subset of the historical events from the first domain and within the start time and the end time,
wherein executing the second query further comprises:
identifying, from a plurality of notifications, a subset of the notifications from the first domain and within the start time and the end time, wherein each historical event from the plurality of historical events comprises a physiological parameter value for a patient at a timestamp;
generating event summary data comprising a first quantity of historical events from the subset of the historical events;
generating notification summary data comprising a second quantity of notifications from the subset of the notifications; and
outputting the event summary data and the notification summary data for presentation in a dashboard user interface.

6. The method of claim 5, wherein the dashboard user interface further comprises:
a first count indicator for the first quantity of historical events,
a second count indicator for the second quantity of notifications,
at least some historical events from the subset of the historical events, and
at least some notifications from the subset of notifications.

7. The method of claim 5, further comprising:
receiving, from the user interface, third user input comprising an updated time range;
determining updated event summary data and updated notification summary data based at least in part on the updated time range; and
outputting the updated event summary data and the updated notification summary data for presentation in an updated dashboard user interface.

8. The method of claim 5, wherein generating the event summary data further comprises:
calculating, from the subset of the historical events, a third quantity of historical events corresponding to a first event category; and
calculating, from the subset of the historical events, a fourth quantity of historical events corresponding to a second event category, wherein the event summary data further comprises the third quantity and the fourth quantity, and wherein the dashboard user interface further comprises:
a third count indicator for the third quantity of historical events, and
a fourth count indicator for the fourth quantity of historical events.

9. The method of claim 5, wherein generating the notification summary data further comprises:
calculating, from the subset of the notifications, a third quantity of notifications corresponding to a first notification category; and
calculating, from the subset of the historical events, a fourth quantity of notifications corresponding to a second notification category, wherein the notification summary data further comprises the third quantity and the fourth quantity, and wherein the dashboard user interface further comprises:
a third count indicator for the third quantity of notifications, and
a fourth count indicator for the fourth quantity of notifications.

10. The method of claim 5, wherein generating the event summary data further comprises:
calculating, from the subset of the historical events, a third quantity of historical events corresponding to a first patient;
calculating, from the subset of the historical events, a fourth quantity of historical events corresponding to a second patient;
determining that the third quantity is greater than the fourth quantity; and
selecting the third quantity for presentation instead of the fourth quantity, wherein the event summary data further comprises the third quantity, and wherein the dashboard user interface further comprises:
a third count indicator for the third quantity of historical events.

11. The method of claim 5, wherein generating the event summary data further comprises:
calculating, from the subset of the historical events, a third quantity of historical events corresponding to a first patient device for a first patient, wherein the event summary data further comprises the third quantity, and wherein the dashboard user interface further comprises:
a third count indicator for the third quantity of historical events.

12. The method of claim 5, wherein the dashboard user interface comprises a patient dashboard user interface for a first patient.

13. A system for generating patient analytics, the system comprising:
data storage that stores a plurality of historical events and a plurality of notifications, wherein each historical event from the plurality of historical events comprises a physiological parameter value for a patient at a timestamp; and
a hardware processor configured to:
receive, from a user interface, first user input comprising a first domain, the first domain indicating a group of patient devices;
receive, from the user interface, second user input comprising a time range, the time range comprising a start time and an end time;
execute (i) a first query comprising input parameters and (ii) and a second query comprising the input parameters, the input parameters comprising the first domain, the start time, and the end time,
wherein executing the first query further comprises:
identifying, from the plurality of historical events, a subset of the historical events from the first domain and within the start time and the end time,
wherein executing the second query further comprises:
identifying, from the plurality of notifications, a subset of the notifications from the first domain and within the start time and the end time;
generate event summary data comprising a first quantity of historical events from the subset of the historical events;
generate notification summary data comprising a second quantity of notifications from the subset of the notifications; and
output the event summary data and the notification summary data for presentation in a dashboard user interface.

14. The system of claim 13, wherein the dashboard user interface further comprises:
a first count indicator for the first quantity of historical events,
a second count indicator for the second quantity of notifications,
at least some historical events from the subset of the historical events, and
at least some notifications from the subset of notifications; and
wherein the hardware processor is further configured to:
receive, from the user interface, third user input comprising an updated time range;
determine updated event summary data and updated notification summary data based at least in part on the updated time range; and
output the updated event summary data and the updated notification summary data for presentation in an updated dashboard user interface.

15. The system of claim 13, wherein a first notification from the plurality of notifications comprises a message that is sent to a clinician device, wherein the message is indicative of a first physiological parameter exceeding an alarm threshold for a predetermined period of time.

16. The system of claim 13, wherein generating the event summary data further comprises:
calculating, from the subset of the historical events, a third quantity of historical events corresponding to a first event category; and
calculating, from the subset of the historical events, a fourth quantity of historical events corresponding to a second event category, wherein the event summary data further comprises the third quantity and the fourth quantity, and wherein the dashboard user interface further comprises:
a third count indicator for the third quantity of historical events, and
a fourth count indicator for the fourth quantity of historical events.

17. The system of claim 16, wherein the first event category comprises at least one of: a clinical event category, a non-clinical event category, or a modifier event category.

18. The system of claim 13, wherein generating the event summary data further comprises:
calculating, from the subset of the notifications, a third quantity of notifications corresponding to a first notification category; and
calculating, from the subset of the historical events, a fourth quantity of notifications corresponding to a second notification category, wherein the notification summary data further comprises the third quantity and the fourth quantity, and wherein the dashboard user interface further comprises:
a third count indicator for the third quantity of notifications, and
a fourth count indicator for the fourth quantity of notifications.

19. The system of claim 13, wherein generating the event summary data further comprises:
calculating, from the subset of the historical events, a third quantity of historical events corresponding to a first patient;
calculating, from the subset of the historical events, a fourth quantity of historical events corresponding to a second patient;
determining that the third quantity is greater than the fourth quantity; and
selecting the third quantity for presentation instead of the fourth quantity, wherein the event summary data further comprises the third quantity, and wherein the dashboard user interface further comprises:
a third count indicator for the third quantity of historical events.

20. The system of claim 13, wherein generating the event summary data further comprises:
calculating, from the subset of the historical events, a third quantity of historical events corresponding to a first patient device for a first patient, wherein the event summary data further comprises the third quantity, and wherein the dashboard user interface further comprises:
a third count indicator for the third quantity of historical events.
